(19) United States
(12) Patent Application Publication
(10) Pub. No.: US 2026/0258021 A1
(43) Pub. Date: Sep. 3, 2026

(54) MONOFLUORINATED COUMARIN FLUOROPHORES AND USES THEREOF

(71) Applicant: Ohio State Innovation Foundation, Columbus, OH (US)

(72) Inventor: Blake PETERSON, Columbus, OH (US)

(21) Appl. No.: 19/161,846

(22) PCT Filed: Mar. 4, 2024
(86) PCT No.: PCT/US2024/018311

§ 371 Date: Sep. 3, 2025

Related U.S. Application Data
(60) Provisional application No. 63/449,721, filed on Mar. 3, 2023.

(51) Int. Cl. C07D 405/12; C07D 407/12; C07D 417/14

(52) U.S. Cl. CPC C07D 405/12; C07D 407/12; C07D 417/14

(57) ABSTRACT

The present disclosure provides compounds having one or more fluorescent moieties, such as compounds of Formula I as described herein, as well as methods of making and using said compounds.

### CROSS-REFERENCE TO RELATED APPLICATIONS

This application claims the benefit of priority to U.S. Provisional Application No. 63/449,721 filed Mar. 3, 2023, the disclosure of which is incorporated herein by reference in its entirety.

### BACKGROUND

Derivatives of 7-hydroxycoumarin-3-carboxylic acid (7OHCCA, 1, FIG. 1) are widely used as blue fluorophores.¹ When the phenol is deprotonated, these compounds generally absorb strongly near 400 nm, allowing efficient excitation with a 405 nm violet laser. This facilitates studies of these compounds in biological systems by confocal microscopy and flow cytometry. Because the pKa of the phenol of 7OHCCA is 7.0-7.5,²⁻⁴ its brightness under physiological conditions can be enhanced by fluorine and other electron withdrawing groups that increase its acidity.⁵ In the fluorophore Pacific Blue (PB, 2),³ fluorination at the 6- and 8-positions reduces the pKa of the phenol to 4.7 (3.7 for PB methyl ester),⁴ providing a superior low molecular weight fluorophore for studies of labeled proteins⁶ and small molecules.⁷ However, when used to study interactions of small molecules with intracellular proteins in live cells, PB derivatives are often efficient substrates of efflux transporters such as p-glycoprotein (MDR1).⁸⁻¹⁰ This high susceptibility to active cellular efflux mechanisms can reduce the ability of related molecular probes to engage intracellular targets in the absence of efflux inhibitors such as verapamil.¹¹

There is a clear need for new fluorophores with enhanced brightness and reduced cellular efflux. This disclosure addresses this as well as other needs.

### SUMMARY

In accordance with the purposes of the disclosed materials and methods, as exemplified and broadly described herein, the disclosed subject matter, in one aspect, relates to compounds, compositions, and methods of making and using said compounds and compositions.

In one aspect, a compound is provided comprising one or more fluorescent moieties selected from:

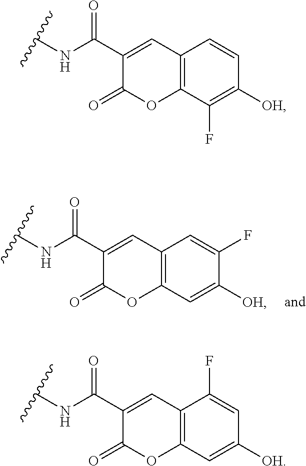 [SMILES: *NC(=O)c1cc2ccc(O)c(F)c2oc1=O ; *NC(=O)c1cc2cc(F)c(O)cc2oc1=O ; *NC(=O)c1cc2c(F)cc(O)cc2oc1=O]

In another aspect, a compound is provided of Formula I

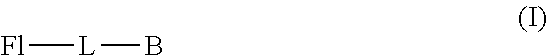

wherein:
Fl is selected from:

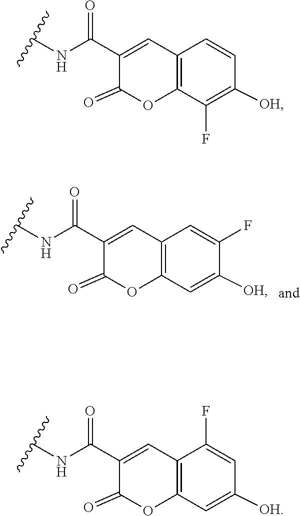 [SMILES: *NC(=O)c1cc2ccc(O)c(F)c2oc1=O ; *NC(=O)c1cc2cc(F)c(O)cc2oc1=O ; *NC(=O)c1cc2c(F)cc(O)cc2oc1=O]

L is selected from a bond or a linker moiety; and
B is a binding moiety.

In another aspect, a method is provided of fluorescently labeling a molecule, wherein the molecule comprises one or more nucleophilic moieties, the method comprising reacting the molecule with a compound selected from:

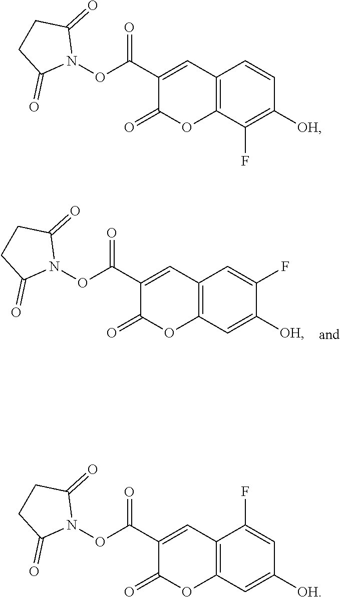 [SMILES: O=C(ON1C(=O)CCC1=O)c1cc2ccc(O)c(F)c2oc1=O ; O=C(ON1C(=O)CCC1=O)c1cc2cc(F)c(O)cc2oc1=O ; O=C(ON1C(=O)CCC1=O)c1cc2c(F)cc(O)cc2oc1=O]

Methods of imaging a sample or a subject are further provided. In some aspects, methods of detecting the presence of a target molecule in a sample or a subject and methods of determining binding affinity between a target molecule and a test compound are also provided.

The details of one or more aspects of the disclosure are set forth in the accompanying drawings and the description below. Other features, objects, and advantages of the disclosure will be apparent from the description, the drawings, and the claims.

### DESCRIPTION OF DRAWINGS

FIG. 1 depicts structures of 7-hydroxycoumarin-3-carboxylic acids (1-5) and related blue-fluorescent derivatives of paclitaxel (Taxol, 6-10).

FIG. 2A depicts the absorbance (10 μM) and fluorescence emission spectra (100 nM) of coumarin amides in aqueous PBS (pH 7.4 for 26-28 and 30, pH 10 for 29, 1% DMSO). Values for λₘₐₓ are listed in Table 1 in the Examples. FIG. 2B depicts the measurements of the pKa of 26-30 (10 μM) by absorbance spectroscopy in aqueous buffers (1% DMSO). pKa values were calculated by non-linear regression (3-parameter fit, GraphPad Prism).

FIGS. 3A and 3B depict the saturation binding of fluorescent taxoids 6-10 to microtubules of living HeLa cells by flow cytometry. Cells in suspension were treated with compounds for 3 h at 37° C. and analyzed by flow cytometry. Cells were treated with probes in the presence (FIG. 3A) and absence (FIG. 3B) of the efflux inhibitor verapamil (100 μM). To measure non-specific binding, paclitaxel (100 μM) was additionally added as a competitor. Concentrations of DMSO stock solutions of fluorescent taxoids 6-10 were measured by absorbance spectroscopy using the molar extinction coefficients of standards 26-30. S/B: Maximal signal-to-background (fold change). [FBS]=4%.

FIG. 4A depicts the absorbance spectroscopy of coumarin hexyl amides 26-28 (9:1 PBS/DMSO, pH 7.4) as spectroscopic standards for probes 8-10. FIG. 4B depicts the determination of molar extinction coefficients (ε_{405 nm}) of 26-28 as previously described in J. Am. Chem. Soc. 2024, 146 (1), 187-200, for 29 and 30 (9:1 PBS/DMSO, pH 7.4). FIG. 4C depicts the determination of quantum yields of 26-29 relative to 30 ((D (pH 7.4)=0.74, 1% DMSO). The integrated fluorescence intensity (Ex. 405 nm, Em. 420-700 nm) was plotted against the maximum absorbance of the sample at the concentration studied (100 nM-6.25 nM) as extrapolated from absorbance measurements at higher concentrations. The slope determined by linear least squares (zero intercept) is proportional to the quantum yield. Spectra were acquired with a BMG LabTech ClarioStar Plus microtiterplate reader.

FIGS. 5A to 5J depict confocal fluorescence (lower images) and differential interference contrast (DIC, upper images) micrographs of living HeLa cells treated with fluorescent taxoids 6-10 (3 h, 37° C.). Gain (610), offset (−1) and laser power (405 nM, 1%) settings are identical in all images to allow accurate comparisons of fluorescence. All of these probes were observed to bind microtubules in living cells. Verapamil (100 μM), added in FIGS. 5F to 5J, substantially enhances the intracellular fluorescence of 6-10, indicating that these compounds are substrates of efflux transporters.

FIGS. 6A and 6B depict a fluorescent probe cellular binding assay (FPCBA) with 6FC-Dasatanib and native DDR1 kinase (IRES-mVenus). (FIG. 6A) Binding of 6FC-Dasatanib to native DDR1 expressed in living HEK293T cells by flow cytometry (2 h, 37° C.). (FIG. 6B) Competition experiments against native DDR1 in living HEK293T cells with the kinase inhibitors ponatinib and dasatinib by flow cytometry. [6FC-Dasatinib]=1 μM. No cytotoxicity was observed as assayed with propidium iodide staining.

Like reference symbols in the various drawings indicate like elements.

### DETAILED DESCRIPTION

The following description of the disclosure is provided as an enabling teaching of the disclosure in its best, currently known aspects. Many modifications and other aspects disclosed herein will come to mind to one skilled in the art to which the disclosed compositions and methods pertain, benefiting from the teachings presented in the descriptions herein and the associated drawings. Therefore, it is understood that the disclosures are not limited to the specific aspects disclosed and that modifications and other aspects are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As apparent to those of skill in the art upon reading this disclosure, each of the individual aspects described and illustrated herein has discrete components and features that may be readily separated from or combined with the features of any of the other several aspects without departing from the scope or spirit of the present disclosure.

Any recited method can be carried out in the order of events recited or any other order that is logically possible. Unless otherwise expressly stated, it is in no way intended that any method or aspect set forth herein be construed as requiring that its steps be performed in a specific order. Accordingly, where a method claim does not explicitly state in the claims or descriptions that the steps are to be limited to a particular order, it is in no way intended that an order be inferred in any respect. This holds for any possible non-express basis for interpretation, including logic concerning arrangement of steps or operational flow, meaning derived from grammatical organization or punctuation, or the number or type of aspects described in the specification.

All publications mentioned herein are incorporated by reference to disclose and describe the methods or materials in connection with which the publications are cited. The publications discussed herein are provided solely for their disclosure before the filing date of the present application. Nothing herein is to be construed as an admission that the present invention is not entitled to antedate such publication by prior invention. Further, the dates of publication provided herein can be different from the actual publication dates, which can require independent confirmation.

It is also to be understood that the terminology herein describes particular aspects only and is not intended to be limiting. Unless defined otherwise, all technical and scientific terms herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Before describing the various aspects of the present disclosure, the following definitions are provided and should be used unless otherwise indicated. Additional terms may be defined elsewhere in the present disclosure.

### Definitions

As used herein, “comprising” is interpreted as specifying the presence of the stated features, integers, steps, or components but does not preclude the presence or addition of one or more features, integers, steps, components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, “consisting essentially of” is intended to include examples encompassed by the term “consisting of”

As used in the specification and the appended claims, the singular forms “a,” “an,” and “the” include plural referents unless the context dictates otherwise.

Ratios, concentrations, amounts, and other numerical data can be expressed herein in a range format. Further, the endpoints of each of the ranges are significant both in relation to the other endpoint and independently of the other endpoint. There are many values disclosed herein, and each value is also disclosed as “about” that particular value in addition to the value itself. For example, if the value “10” is disclosed, then “about 10” is also disclosed. Ranges can be expressed herein as from “about” one particular value and to “about” another particular value. Similarly, when values are expressed as approximations, using the antecedent “about,” the particular value forms a further aspect. For example, if the value “about 10” is disclosed, then “10” is also disclosed.

When a range is expressed, a further aspect includes from the one particular value and to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x,’ ‘about y,’ and ‘about z’ as well as the ranges of ‘less than x,’ ‘less than y.’ and ‘less than z.’ Likewise, the phrase ‘about x, y, z, or greater’ should be interpreted to include the specific ranges of ‘about x,’ ‘about y,’ and ‘about z’ as well as the ranges of ‘greater than x,’ greater than y,’ and ‘greater than z.’ In addition, the phrase “about ‘x’ to ‘y’,” where ‘x’ and ‘y’ are numerical values, includes “about ‘x’ to about ‘y’.”

Such a range format is used for convenience and brevity and, thus, should be interpreted flexibly to include not only the numerical values explicitly recited as the limits of the range but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5% but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, amounts, sizes, formulations, parameters, and other quantities and characteristics are not and need not be exact but may be approximate, larger or smaller, as desired, reflecting tolerances, conversion factors, rounding, measurement error, and the like, and other factors known to those of skill in the art such that equivalent results or effects are obtained. In some circumstances, the value that provides equivalent results or effects cannot be reasonably determined. In such cases, as used herein, “about” and “at or about” mean the nominal value indicated±10% variation unless otherwise indicated or inferred. In general, an amount, size, formulation, parameter, or other quantity or characteristic is “about,” “approximate,” or “at or about,” whether or not expressly stated to be such. Where “about,” “approximate,” or “at or about” is used before a quantitative value, the parameter also includes the specific quantitative value itself unless expressly stated otherwise.

As used herein, “optional” or “optionally” means that the subsequently described event or circumstance can or cannot occur. The description includes instances where said event or circumstance occurs and those where it does not.

As used herein, the term “substantially” means that the subsequently described event or circumstance completely occurs or that the subsequently described event or circumstance generally, typically, or approximately occurs.

Still further, the term “substantially” can, in some aspects, refer to at least about 90%, at least about 91%, at least about 92%, at least about 93%, at least about 94%, at least about 95%, at least about 96%, at least about 97%, at least about 98%, at least about 99%, or about 100% of the stated property, component, composition, or other condition for which substantially is used to characterize or otherwise quantify an amount.

As used herein, the term “substantially,” in, for example, the context “substantially identical” or “substantially similar,” refers to a method or a system, or a component that is at least about 90%, at least about 91%, at least about 92%, at least about 93%, at least about 94%, at least about 95%, at least about 96%, at least about 97%, at least about 98%, at least about 99%, or about 100% by similar to the method, system, or the component it is compared to.

### Chemical Definitions

Compounds are described using standard nomenclature. Unless defined otherwise, all technical and scientific terms used herein have the same meaning as is commonly understood by one of skill in the art to which this invention belongs.

The compounds described herein include enantiomers, mixtures of enantiomers, diastereomers, tautomers, racemates, and other isomers, such as rotamers, as if each is specifically described unless otherwise indicated or otherwise excluded by context. It is to be understood that the compounds provided herein may contain chiral centers. Such chiral centers may be of either the (R-) or (S-) configuration. The compounds provided herein may either be enantiomerically pure or diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A dash (“-”) that is not between two letters or symbols is used to indicate a point of attachment for a substituent. For example, —(C═O)NH₂ is attached through the carbon of the keto (C═O) group.

The term “substituted,” as used herein, means that any one or more hydrogens on the designated atom or group are replaced with a moiety selected from the indicated group, provided that the designated atom's normal valence is not exceeded and the resulting compound is stable. For example, when the substituent is oxo (i.e., ═O) then two hydrogens on the atom are replaced. For example, a pyridyl group substituted by oxo is a pyridine. Combinations of substituents and/or variables are permissible only if such combinations result in stable compounds or useful synthetic intermediates. A stable active compound refers to a compound that can be isolated and/or can be formulated into a form with a shelf life of at least one month. A stable manufacturing intermediate or precursor to an active compound is stable if it does not degrade within the period needed for reaction or other use. A stable moiety or substituent group is one that does not degrade, react, or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the invention and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

The terms for various functional groups as used herein are not intended to be limited to monovalent radicals and may include polyvalent radical groups as appropriate, such as divalent, trivalent, tetravalent, pentavalent, and hexavalent groups, and the like, based on the position and location of such groups in the compounds described herein as would be readily understood by the skilled person.

As used herein, the symbol “

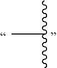

” (which hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example, “

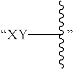

” indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or “

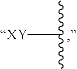

” infers that when R³ is “XY,” the point of attachment bond is the same bond as the bond by which R³ is depicted as being bonded to CH₃.

“Alkyl” is a straight chain or branched saturated aliphatic hydrocarbon group. In certain aspects, the alkyl is C₁-C₂, C₁-C₃, or C₁-C₆ (i.e., the alkyl chain can be 1, 2, 3, 4, 5, or 6 carbons in length). The specified ranges, as used herein, indicate an alkyl group with the length of each member of the range described as an independent species. For example, C₁-C₆alkyl, as used herein, indicates an alkyl group having from 1, 2, 3, 4, 5, or 6 carbon atoms and is intended to mean that each of these is described as an independent species, and C₁-C₄alkyl, as used herein, indicates an alkyl group having from 1, 2, 3, or 4 carbon atoms and is intended to mean that each of these is described as an independent species. When C₀-Cₙalkyl is used herein in conjunction with another group, for example (C₃-C₇cycloalkyl)C₀-C₄alkyl, or —C₀-C₄(C₃-C₇cycloalkyl), the indicated group, in this case cycloalkyl, is either directly bound by a single covalent bond (C₀alkyl), or attached by an alkyl chain, in this case 1, 2, 3, or 4 carbon atoms. Alkyls can also be attached via other groups, such as heteroatoms, as in —O—C₀-C₄alkyl(C₃-C₇cycloalkyl). Examples of alkyl include but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In some aspects, the alkyl group is optionally substituted as described herein.

“Cycloalkyl” is a saturated or partially unsaturated mono- or multi-cyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. In some aspects, the cycloalkyl group is optionally substituted as described herein.

“Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₆alkenyl (i.e., having 2, 3, 4, 5, or 6 carbons). The specified ranges as used herein indicate an alkenyl group having each member of the range described as an independent species, as described above for the alkyl moiety. Examples of alkenyl include but are not limited to, ethenyl and propenyl. In one aspect, the alkenyl group is optionally substituted as described herein.

“Alkynyl” is a straight or branched chain aliphatic hydrocarbon group having one or more carbon-carbon triple bonds that may occur at any stable point along the chain, for example, C₂-C₄alkynyl or C₂-C₆alkynyl (i.e., having 2, 3, 4, 5, or 6 carbons). The specified ranges, as used herein, indicate an alkynyl group, with each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one aspect, the alkynyl group is optionally substituted as described herein.

“Alkoxy” is an alkyl group, as defined above, covalently bound through an oxygen bridge (—O—). Examples of alkoxy include but are not limited to, methoxy, ethoxy, n-propoxy, isopropoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or “thioalkyl” group is an alkyl group as defined above with the indicated number of carbon atoms covalently bound through a sulfur bridge (—S—).

“Alkanoyl” is an alkyl group, as defined above, covalently bound through a carbonyl (C═O) bridge. The carbonyl carbon is included in the number of carbons. For example, C₂alkanoyl is a CH₃(C═O)— group. In one aspect, the alkanoyl group is optionally substituted as described herein.

“Halo” or “halogen” indicates, independently, any of fluoro, chloro, bromo or iodo.

“Aryl” indicates an aromatic group containing only carbon in the aromatic ring or rings. In one aspect, the aryl group contains 1 to 3 separate or fused rings and is 6 to 14 or 18 ring atoms, without heteroatoms as ring members. When indicated, such aryl groups may be further substituted with carbon or non-carbon atoms or groups. Such substitution may include fusion to a 4- to 7- or 5- to 7-membered saturated or partially unsaturated cyclic group that optionally contains 1, 2, or 3 heteroatoms independently selected from N, O, B, P, Si, and S to form, for example, a 3,4-methylenedioxyphenyl group. Aryl groups include, for example, phenyl and naphthyl, including 1-naphthyl and 2-naphthyl. In one aspect, aryl groups are pendant. An example of a pendant ring is a phenyl group substituted with a phenyl group. In one aspect, the aryl group is optionally substituted as described herein.

The term “heterocycle” refers to saturated and partially saturated heteroatom-containing ring radicals, where the heteroatoms may be selected from N, O, and S. The term heterocycle includes monocyclic 3-12 members rings, as well as bicyclic 5-16 membered ring systems (which can include fused, bridged, or spiro bicyclic ring systems). It does not include rings containing —O—O—, —O—S—, and —S—S— portions. Examples of saturated heterocycle groups including saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4- to 6-membered monocyclic groups containing 1 to 2 oxygen atoms and 1 to 3 nitrogen atoms [e.g., morpholinyl]; and saturated 3- to 6-membered heteromonocyclic groups containing 1 to 2 sulfur atoms and 1 to 3 nitrogen atoms [e.g., thiazolidinyl]. Examples of partially saturated heterocycle radicals include, but are not limited, dihydrothienyl, dihydropyranyl, dihydrofuryl, and dihydrothiazolyl. Examples of partially saturated and saturated heterocycle groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3,-dihydro-1H-benzo[d]isothazol-6-yl, dihydropyranyl, dihydrofuryl, and dihydrothiazolyl. Bicyclic heterocycle includes groups wherein the heterocyclic radical is fused with an aryl radical wherein the point of attachment is the heterocycle ring. Bicyclic heterocycle also includes heterocyclic radicals that are fused with a carbocyclic radical. Representative examples include but are not limited to, partially unsaturated condensed heterocyclic groups containing 1 to 5 nitrogen atoms, for example, indoline and isoindoline, partially unsaturated condensed heterocyclic groups containing 1 to 2 oxygen atoms and 1 to 3 nitrogen atoms, partially unsaturated condensed heterocyclic groups containing 1 to 2 sulfur atoms and 1 to 3 nitrogen atoms, and saturated condensed heterocyclic groups containing 1 to 2 oxygen or sulfur atoms.

“Heteroaryl” refers to a stable monocyclic, bicyclic, or multicyclic aromatic ring that contains from 1 to 4, or in some aspects, 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 4, or in some aspects from 1 to 3 or from 1 to 2, heteroatoms selected from N, O, S, B, or P, with remaining ring atoms being carbon. In one aspect, the only heteroatom is nitrogen. In one aspect, the only heteroatom is oxygen. In one aspect, the only heteroatom is sulfur. Monocyclic heteroaryl groups typically have from 5 to 6 ring atoms. In some aspects, bicyclic heteroaryl groups are 8- to 10-membered heteroaryl groups, that is groups containing 8 or 10 ring atoms in which one 5-, 6-, or 7-membered aromatic ring which contains from 1 to 4 heteroatoms selected from N, O, S, B, or P is fused to a second aromatic or non-aromatic ring, wherein the point of attachment is an aromatic ring. When the total number of S and O atoms in the heteroaryl ring exceeds 1, these heteroatoms are not adjacent to one another within the ring. In one aspect, the total number of S and O atoms in the heteroaryl ring is not more than 2. In another aspect, the total number of S and O atoms in the heteroaryl ring is not more than 1. Examples of heteroaryl groups include, but are not limited to, pyridinyl, imidazolyl, imidazopyridinyl, pyrimidinyl, pyrazolyl, thiazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

As used herein, substantially pure means sufficiently homogeneous to appear free of readily detectable impurities as determined by standard methods of analysis, such as thin layer chromatography (TLC), nuclear magnetic resonance (NMR), gel electrophoresis, high-performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modem methods for purification of the compounds to produce substantially chemically pure compounds are known to those skilled in the art. A substantially chemically pure compound may, however, be a mixture of stereoisomers.

The present disclosure also includes compounds with at least one desired isotopic substitution of an atom at an amount above the natural abundance of the isotope, i.e., enriched.

Examples of isotopes that can be incorporated into compounds of the present disclosure include isotopes of hydrogen, carbon, nitrogen, oxygen, phosphorous, fluorine, and chlorine, such as ²H, ³H, ¹¹C, ¹³C, ¹⁵N, ¹⁷O ¹⁸F, ³¹P, ³²P, ³⁵S, ³⁶Cl, and ¹²⁵I, respectively. Isotopically labeled compounds of this invention can generally be prepared by carrying out the procedures disclosed herein by substituting a readily available isotopically labeled reagent for a non-isotopically labeled reagent.

By way of general example and without limitation, isotopes of hydrogen, for example, deuterium (H) and tritium (H), may optionally be used anywhere in described structures that achieve the desired result. Alternatively, or in addition, isotopes of carbon, e.g., ¹³C and ¹⁴C, may be used.

Isotopic substitutions, for example, deuterium substitutions, can be partial or complete. Partial deuterium substitution means that at least one hydrogen is substituted with deuterium. In certain aspects, the isotope is 80, 85, 90, 95, or 99% or more enriched in an isotope at any location of interest. In some aspects, deuterium is 80, 85, 90, 95, or 99% enriched at a desired location. Unless otherwise stated, the enrichment at any point is above natural abundance and, in an aspect, is enough to alter a detectable property of the compounds.

### Compounds

In some aspects, a compound is provided comprising one or more fluorescent moieties selected from:

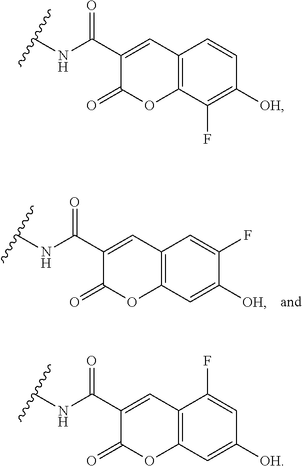 [SMILES: *NC(=O)c1cc2ccc(O)c(F)c2oc1=O ; *NC(=O)c1cc2cc(F)c(O)cc2oc1=O ; *NC(=O)c1cc2c(F)cc(O)cc2oc1=O]

In some aspects, the compound comprising the above one or more fluorescent moieties comprises a protein, a peptide, an antibody, an antigen, a receptor, a nucleic acid, a nucleotide, a nucleotide derivative, a therapeutic agent, a small molecule, a synthetic oligomer, a synthetic polymer, a hormone, a lymphokine, a cytokine, a toxin, a ligand, a carbohydrate, a sugar, an oligosaccharide, a polysaccharide, a fatty acid, or the like.

In some aspects, the compound comprising the above one or more fluorescent moieties is a fluorescent probe.

In another aspect, a compound is provided of Formula I:

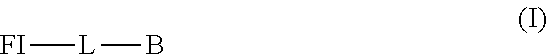

wherein:
Fl is selected from:

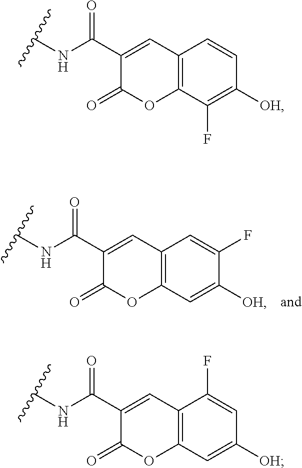 [SMILES: *NC(=O)c1cc2ccc(O)c(F)c2oc1=O ; *NC(=O)c1cc2cc(F)c(O)cc2oc1=O ; *NC(=O)c1cc2c(F)cc(O)cc2oc1=O]

L is selected from a bond or a linker moiety; and
B is a binding moiety.

In some aspects, Fl is

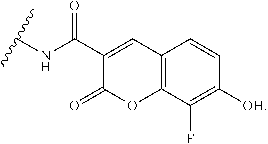 [SMILES: *NC(=O)c1cc2ccc(O)c(F)c2oc1=O]

In some aspects, Fl is

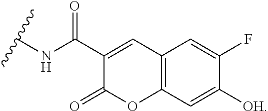 [SMILES: *NC(=O)c1cc2cc(F)c(O)cc2oc1=O]

In some aspects, Fl is

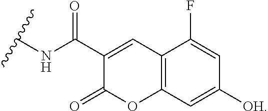 [SMILES: *NC(=O)c1cc2c(F)cc(O)cc2oc1=O]

In some aspects of Formula I, L is a bond, i.e., the Fl and B moieties are directly attached. In some aspects of Formula I, L comprises a linker moiety. The linker moiety is a chemically stable multivalent (e.g., bivalent) group that attaches Fl to B. Linker as described herein can be used in either direction, i.e., either the left end is linked to Fl and the right end to B, or the left end is linked to B and the right end to Fl.

In some aspects, the linker moiety is a chain of 2 to 14, 15, 16, 17, 18, 19, or 20 or more carbon atoms, of which one or more carbons can be optionally replaced by a heteroatom such as O, N, S, or P.

In some aspects, the chain has 2, 3, 4, 5, 6, 7, 8, 9, 10, 11, 12, 13, 14, 15, 16, 18, 19, 19, or 20 contiguous atoms. For example, the chain may include 1 or more ethylene glycol units that can be contiguous, partially contiguous, or non-contiguous (for example, 2, 3, 4, 5, 6, 7, 8, 9, 10, 11, or 12 ethylene glycol units).

In some aspects, the chain has at least 1, 2, 3, 4, 5, 6, 7, or 8 contiguous units which can be branched and which can be independently alkyl, aryl, heteroaryl, alkenyl, or alkynyl, cycloalkyl, or heterocycloalkyl substituents.

In some aspects, the linker moiety can include or be comprised of one or more ethylene glycol, propylene glycol, lactic and/or glycolic acid units. Block and random lactic acid-co-glycolic acid moieties, as well as ethylene glycol and propylene glycol, are known in the art and can be modified to obtain the desired half-life and hydrophilicity. In certain aspects, these units can be flanked or interspersed with other moieties, such as for example alkyl, heteroalkyl, aryl, heteroaryl, cycloalkyl, heterocycloalkyl, etc., as desired to achieve the appropriate properties.

In some aspects, the linker moiety is an optionally substituted (poly)ethylene glycol having at least 1, at least 2, at least 3, at least 4, at least 5, at least 6, at least 7, at least 8, at least 9, at least 10, or more, ethylene glycol units, or optionally substituted alkyl groups interspersed with optionally substituted O, N, S, P or Si atoms.

In some aspects, the linker moiety is flanked, substituted, or interspersed with an alkyl, cycloalkyl, heterocycloalkyl, aryl, or heteroaryl group.

In some aspects, the linker moiety may be asymmetric or symmetric.

In some aspects, the linker moiety can be a non-linear chain and can be, or include, cycloalkyl, heterocycloalkyl, aryl, or heteroaryl cyclic moieties.

In some aspects, the linker moiety is selected from L1:

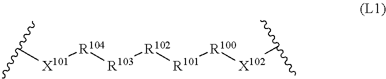

In some aspects, the linker moiety is selected from the group consisting of a moiety of Formula L1, Formula L2, Formula L3, Formula L4, Formula L5, Formula L6, Formula L7, Formula L8, Formula L9, or Formula L10:

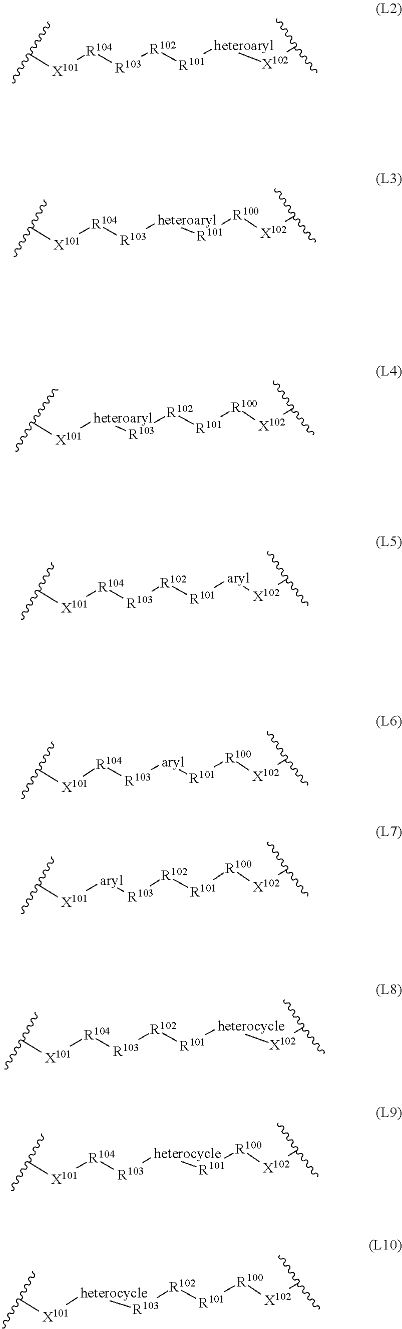

wherein:
X¹⁰¹ and X¹⁰² are independently at each occurrence selected from a bond, aryl, heteroaryl, cycloalkyl, heterocycle, NR¹³⁰C(R¹³⁰)₂, O, C(O), and S;
R¹⁰⁰, R¹⁰¹, R¹⁰², R¹⁰³, and R¹⁰⁴ are independently at each occurrence selected from the group consisting of a bond, alkyl, —C(O)—, —C(O)O—, —OC(O)—, —SO₂—, —S(O)—, C(S)—, —C(O)NR¹³⁰—, —NR¹³⁰C(O)—, —O—, —S—, —NR¹³⁰—, —C(R¹³⁰R¹³⁰)—, —P(O)(OR¹⁰⁶))—, —R(O)(OR¹⁰⁶)—, alkenyl, alkynyl, haloalkyl, alkoxy, aryl, heterocycloalkyl, cycloalkyl, heteroaryl, lactic acid, or glycolic acid, each of which may be optionally substituted with one or more (for example, 1, 2, 3, or 4) substituents independently selected from R¹⁴⁰;
R¹⁰⁶ is independently at each occurrence selected from the group consisting of hydrogen, alkyl, arylalkyl, heteroarylalkyl, alkenyl, alkynyl, aryl, heteroaryl, cycloalkyl, or heterocycloalkyl;
R¹³⁰ is independently as each occurrence selected from the group consisting of hydrogen, alkyl, cycloalkyl, heterocycloalkyl, aryl, heteroaryl, —C(O)H, —C(O)OH, —C(O)alkyl, —C(O)Oalkyl, —C(O)(cycloalkyl, heterocycloalkyl, aryl, or heteroaryl), —C(O)O(cycloalkyl, heterocycloalkyl, aryl, or heteroaryl), alkenyl, or alkynyl; and
R¹⁴⁰ is independently at each occurrence selected from the group consisting of hydrogen, alkyl, alkenyl, alkynyl, fluoro, bromo, chloro, hydroxyl, alkoxy, azide, amino, cyano, —NH(alkyl, cycloalkyl, heterocyloalkyl, aryl, or heteroaryl), —N(independently alkyl, cycloalkyl, heterocycloalkyl, aryl, or heteroaryl), —NHSO₂(alkyl, cycloalkyl, heterocycloalkyl, aryl, or heteroaryl), —N(alkyl, cycloalkyl, heterocycloalkyl, aryl, or heteroaryl)SO₂alkyl, —NHSO₂alkenyl, —N(alkyl)SO₂alkenyl, —NHSO₂alkynyl, —N(alkyl)SO₂alkynyl, haloalkyl, cycloalkyl, heterocycloalkyl, aryl, or heteroaryl.

In some aspects, R¹⁰⁰, R¹⁰¹, R¹⁰², R¹⁰³, and R¹⁰⁴ within the linker moiety are selected in such a manner that no two —C(═O)— moieties are adjected to each other; no two —O— or —NH— moieties are adjacent to each other; and/or no moieties are otherwise selected in an order such that an unstable molecule results (as defined as producing a molecule that has a shelf life at ambient temperature of less than about six months, five months, or four months) due to decomposition caused by the selection and order of R¹⁰⁰, R¹⁰¹, R¹⁰², R¹⁰³, and R¹⁰⁴.

The following are non-limiting exemplary of linker moieties and/or moieties that comprise linker moieties in whole or in part that can be used in this invention. Based on this elaboration, those skilled in the art will understand how to use the full breadth of linker moieties that will accomplish the goal of the invention.

Non-limiting examples of moieties that may comprise the linker moiety, either in whole or in part, include, but are not limited to:
a bond; —C(═O)—; —C═C—; —NH—; —N(CH₃)—; —O—; —CH₂—; —(CH₂)₂—; —(CH₂)₃—; —(CH₂)₄—; —(CH₂)₅—; —(CH₂)₆—; —(CH₂)₇—; —(CH₂)₈—; —(CH₂)₉—; —(CH₂)₁₀—; —NH(C═O)—; —C(═O)NH—; —C(═O)CH₂—; —C(═O)(CH₂)₂—; —C(═O)(CH₂)₃—; —C(═O)(CH₂)₄—; —C(═O)(CH₂)₅—; —C(═O)(CH₂)₆—; —CH₂C(═O)—; —(CH₂)₂C(═O)—; —(CH₂)₃C(═O)—; —(CH₂)₄C(═O)—; —(CH₂)₅C(═O)—; —(CH₂)₆C(═O)—; —CH₂NH—; —(CH₂)₂NH—; —(CH₂)₃NH—; —(CH₂)₄NH—; —(CH₂)₅NH—; —(CH₂)₆NH—; —NHCH₂—; —NH(CH₂)₂—; —NH(CH₂)₃—; —NH(CH₂)₄—; —NH(CH₂)₅—; —NH(CH₂)₆—; —CH₂O—; —(CH₂)₂O—; —(CH₂)₃O—; —(CH₂)₄O—; —(CH₂)₅O—; —(CH₂)₆O—; —OCH₂—; —O(CH₂)₂—; —O(CH₂)₃—; —O(CH₂)₄—; —O(CH₂)₅—; —O(CH₂)₆—;

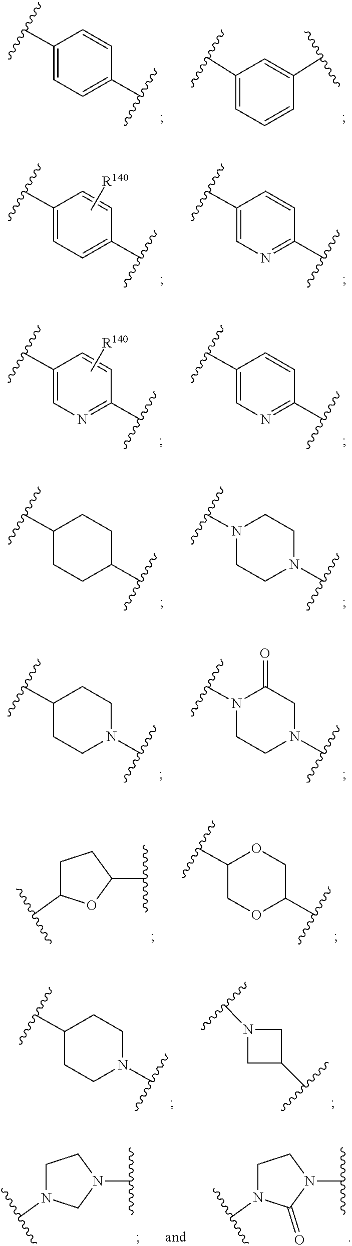

Further non-limiting examples of moieties that may comprise the linker moiety, either in whole or in part, include, but are not limited to:

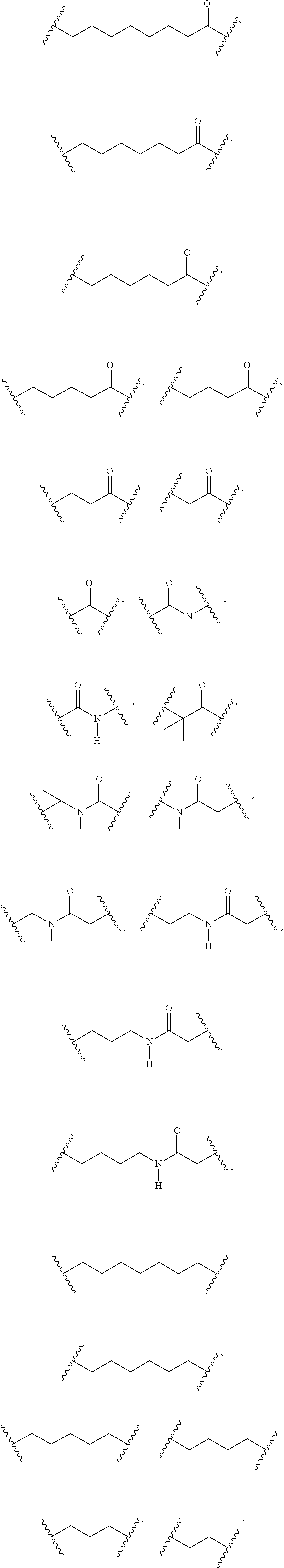

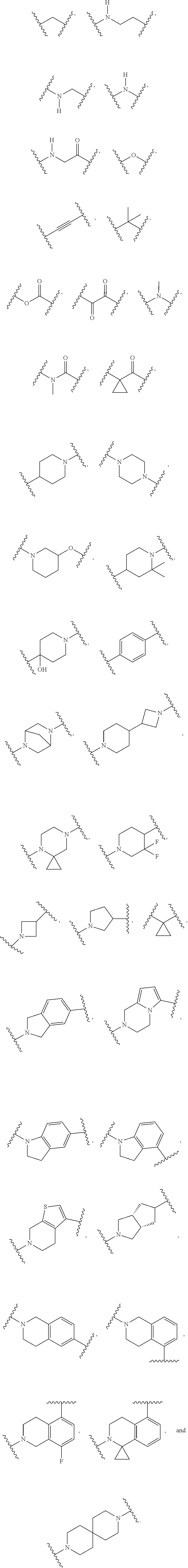

In some aspects, the linker moiety may comprise, either in whole or in part, a moiety selected from:

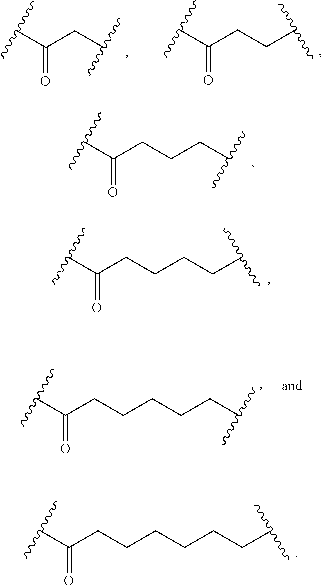 [SMILES: *CC(*)=O ; *CCC(*)=O ; *CCCC(*)=O ; *CCCCC(*)=O ; *CCCCCC(*)=O ; *CCCCCCC(*)=O]

In some aspects, the linker moiety may comprise, either in whole or in part, a moiety selected from:

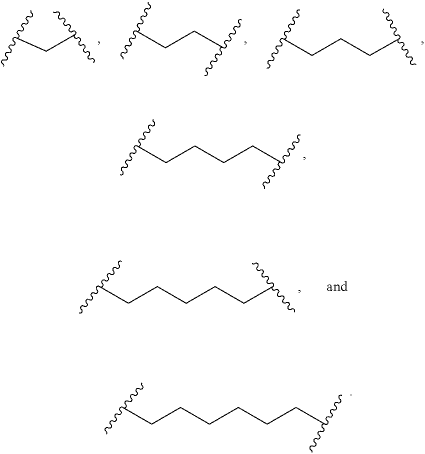

In some aspects, the linker moiety may comprise, either in whole or in part, a moiety selected from:

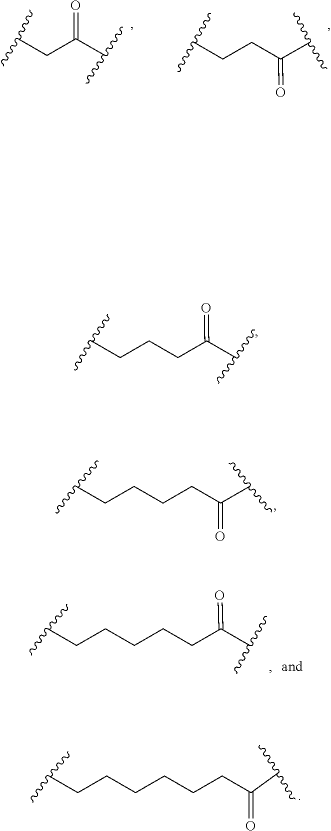 [SMILES: *CC(*)=O ; *CCC(*)=O ; *CCCC(*)=O ; *CCCCC(*)=O ; *CCCCCC(*)=O ; *CCCCCCC(*)=O]

In some aspects, the linker moiety may comprise, either in whole or in part,

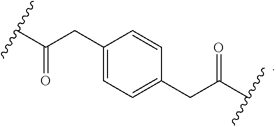 [SMILES: *C(=O)Cc1ccc(CC(*)=O)cc1]

In some aspects, the linker moiety may comprise, either in whole or in part,

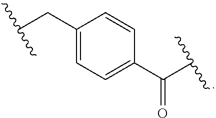 [SMILES: *Cc1ccc(C(*)=O)cc1]

In some aspects, the linker moiety may comprise, either in whole or in part, a moiety

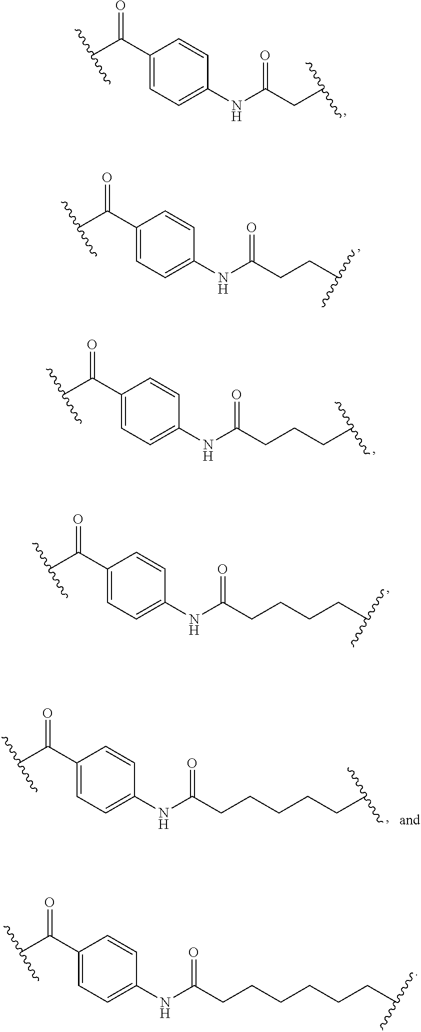 [SMILES: *CC(=O)Nc1ccc(C(*)=O)cc1 ; *CCC(=O)Nc1ccc(C(*)=O)cc1 ; *CCCC(=O)Nc1ccc(C(*)=O)cc1 ; *CCCCC(=O)Nc1ccc(C(*)=O)cc1 ; *CCCCCC(=O)Nc1ccc(C(*)=O)cc1 ; *CCCCCCC(=O)Nc1ccc(C(*)=O)cc1]

In some aspects, the linker moiety may comprise, either in whole or in part, a moiety selected from:

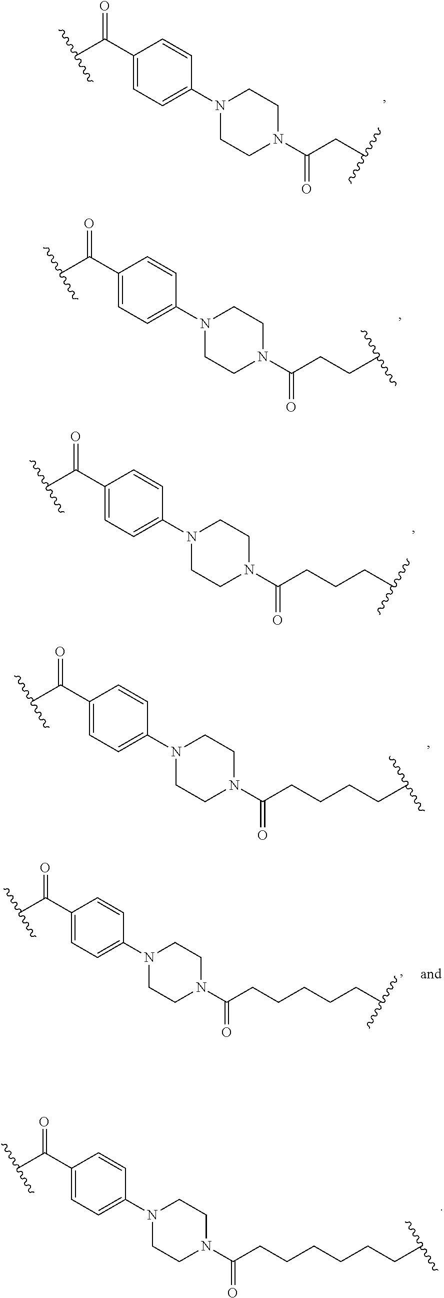 [SMILES: *CC(=O)N1CCN(c2ccc(C(*)=O)cc2)CC1 ; *CCC(=O)N1CCN(c2ccc(C(*)=O)cc2)CC1 ; *CCCC(=O)N1CCN(c2ccc(C(*)=O)cc2)CC1 ; *CCCCC(=O)N1CCN(c2ccc(C(*)=O)cc2)CC1 ; *CCCCCC(=O)N1CCN(c2ccc(C(*)=O)cc2)CC1 ; *CCCCCCC(=O)N1CCN(c2ccc(C(*)=O)cc2)CC1]

In some aspects, the linker moiety may comprise, either in whole or in part, a moiety selected from:

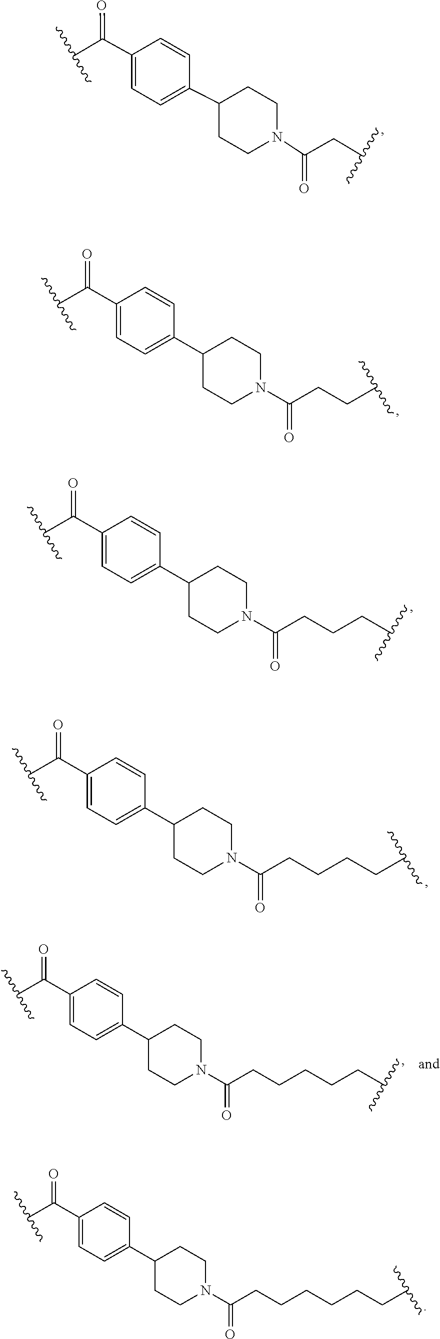 [SMILES: *CC(=O)N1CCC(c2ccc(C(*)=O)cc2)CC1 ; *CCC(=O)N1CCC(c2ccc(C(*)=O)cc2)CC1 ; *CCCC(=O)N1CCC(c2ccc(C(*)=O)cc2)CC1 ; *CCCCC(=O)N1CCC(c2ccc(C(*)=O)cc2)CC1 ; *CCCCCC(=O)N1CCC(c2ccc(C(*)=O)cc2)CC1 ; *CCCCCCC(=O)N1CCC(c2ccc(C(*)=O)cc2)CC1]

In some aspects, the linker moiety may comprise, either in whole or in part, a moiety selected from:

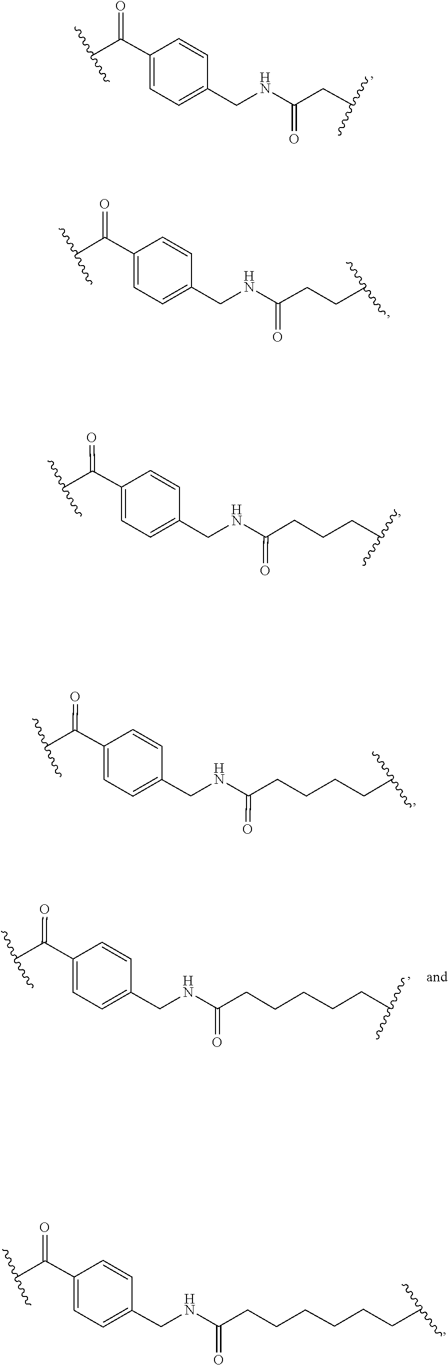 [SMILES: *CC(=O)NCc1ccc(C(*)=O)cc1 ; *CCC(=O)NCc1ccc(C(*)=O)cc1 ; *CCCC(=O)NCc1ccc(C(*)=O)cc1 ; *CCCCC(=O)NCc1ccc(C(*)=O)cc1 ; *CCCCCC(=O)NCc1ccc(C(*)=O)cc1 ; *CCCCCCC(=O)NCc1ccc(C(*)=O)cc1]

In some aspects, the linker moiety may comprise, either in whole or in part, a moiety selected from:

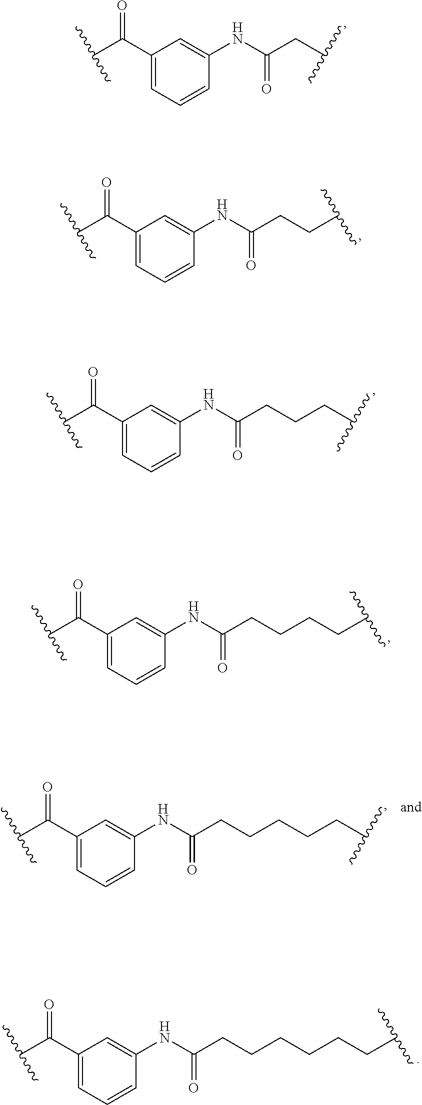 [SMILES: *CC(=O)Nc1cccc(C(*)=O)c1 ; *CCC(=O)Nc1cccc(C(*)=O)c1 ; *CCCC(=O)Nc1cccc(C(*)=O)c1 ; *CCCCC(=O)Nc1cccc(C(*)=O)c1 ; *CCCCCC(=O)Nc1cccc(C(*)=O)c1 ; *CCCCCCC(=O)Nc1cccc(C(*)=O)c1]

In some aspects, the linker moiety may comprise, either in whole or in part, a moiety selected from:

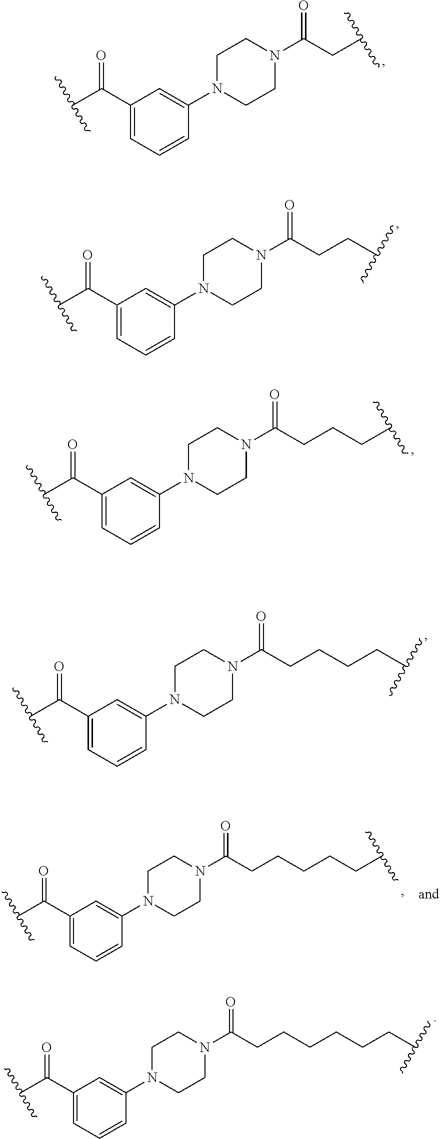 [SMILES: *CC(=O)N1CCN(c2cccc(C(*)=O)c2)CC1 ; *CCC(=O)N1CCN(c2cccc(C(*)=O)c2)CC1 ; *CCCC(=O)N1CCN(c2cccc(C(*)=O)c2)CC1 ; *CCCCC(=O)N1CCN(c2cccc(C(*)=O)c2)CC1 ; *CCCCCC(=O)N1CCN(c2cccc(C(*)=O)c2)CC1 ; *CCCCCCC(=O)N1CCN(c2cccc(C(*)=O)c2)CC1]

In some aspects, the linker moiety may comprise, either in whole or in part, a moiety selected from:

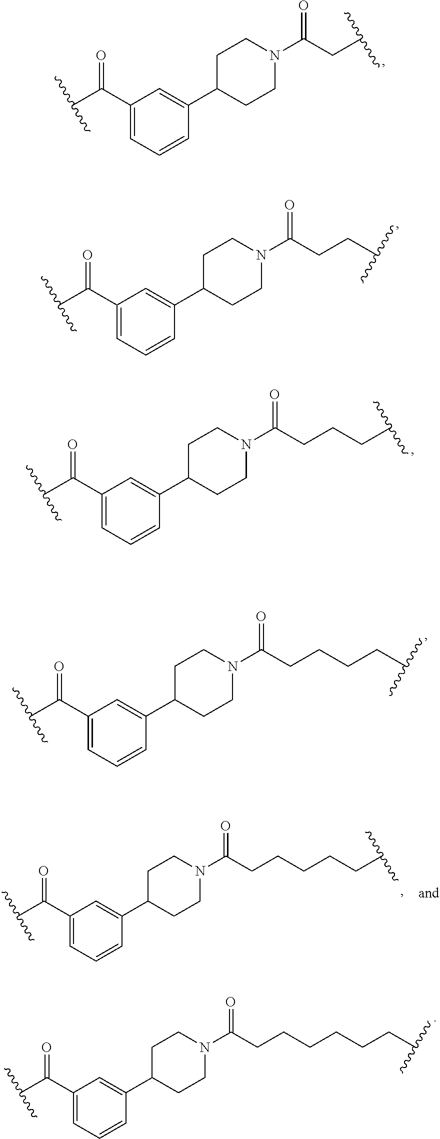 [SMILES: *CC(=O)N1CCC(c2cccc(C(*)=O)c2)CC1 ; *CCC(=O)N1CCC(c2cccc(C(*)=O)c2)CC1 ; *CCCC(=O)N1CCC(c2cccc(C(*)=O)c2)CC1 ; *CCCCC(=O)N1CCC(c2cccc(C(*)=O)c2)CC1 ; *CCCCCC(=O)N1CCC(c2cccc(C(*)=O)c2)CC1 ; *CCCCCCC(=O)N1CCC(c2cccc(C(*)=O)c2)CC1]

In some aspects, the linker moiety may comprise, either in whole or in part, a moiety selected from:

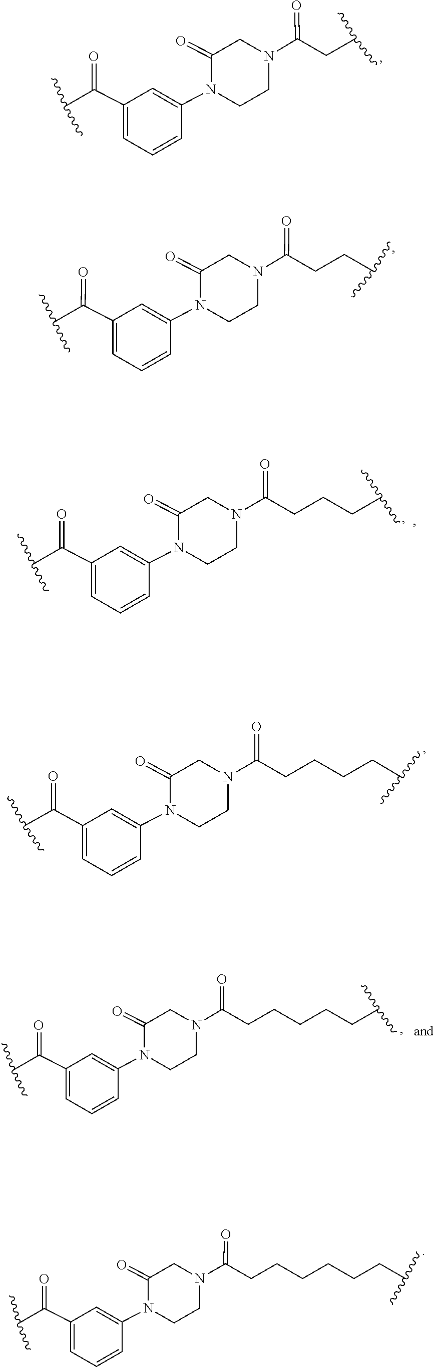 [SMILES: *CC(=O)N1CCN(c2cccc(C(*)=O)c2)C(=O)C1 ; *CCC(=O)N1CCN(c2cccc(C(*)=O)c2)C(=O)C1 ; *CCCC(=O)N1CCN(c2cccc(C(*)=O)c2)C(=O)C1 ; *CCCCC(=O)N1CCN(c2cccc(C(*)=O)c2)C(=O)C1 ; *CCCCCC(=O)N1CCN(c2cccc(C(*)=O)c2)C(=O)C1 ; *CCCCCCC(=O)N1CCN(c2cccc(C(*)=O)c2)C(=O)C1]

In some aspects, the linker moiety may comprise, either in whole or in part, a moiety selected from:

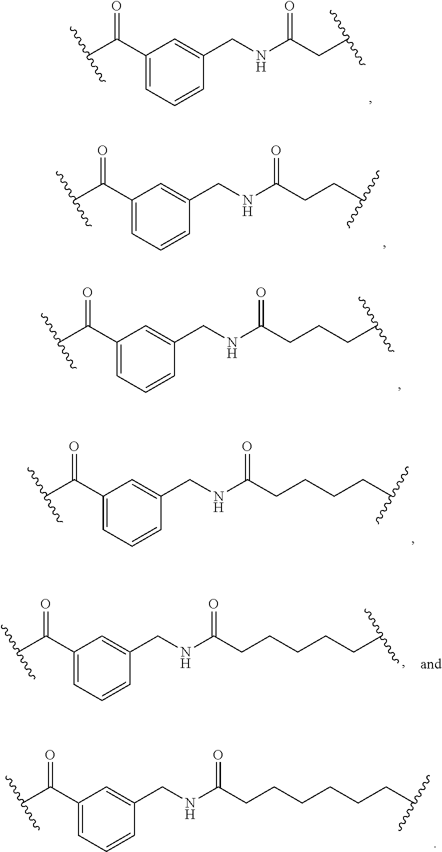 [SMILES: *CC(=O)NCc1cccc(C(*)=O)c1 ; *CCC(=O)NCc1cccc(C(*)=O)c1 ; *CCCC(=O)NCc1cccc(C(*)=O)c1 ; *CCCCC(=O)NCc1cccc(C(*)=O)c1 ; *CCCCCC(=O)NCc1cccc(C(*)=O)c1 ; *CCCCCCC(=O)NCc1cccc(C(*)=O)c1]

In some aspects, the linker moiety may comprise, either in whole or in part, a moiety selected from:

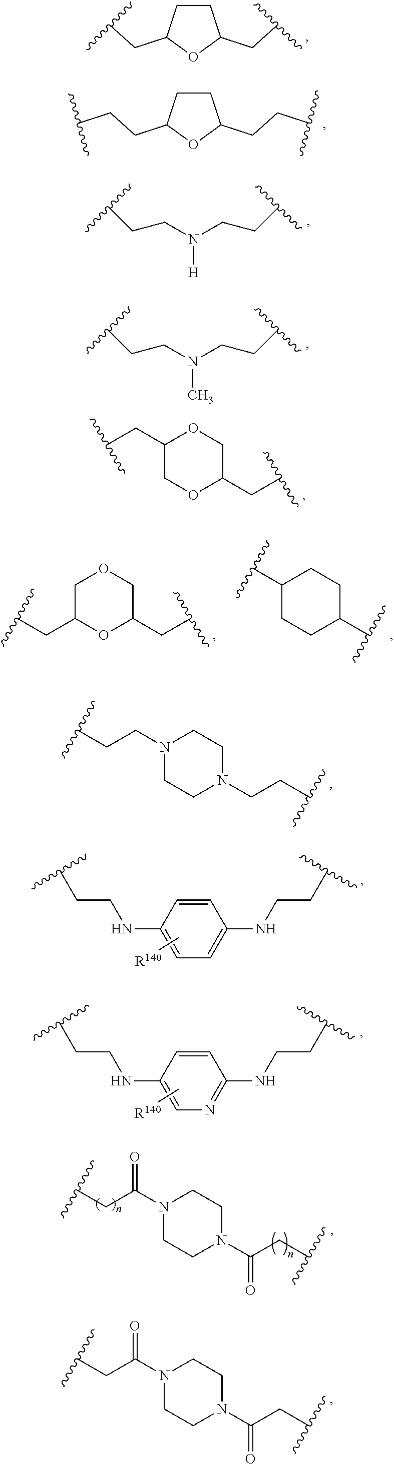

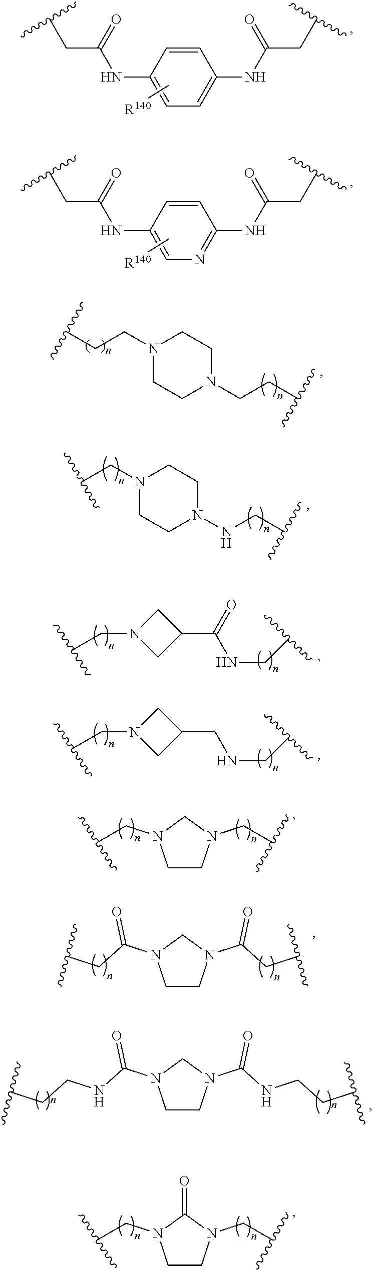

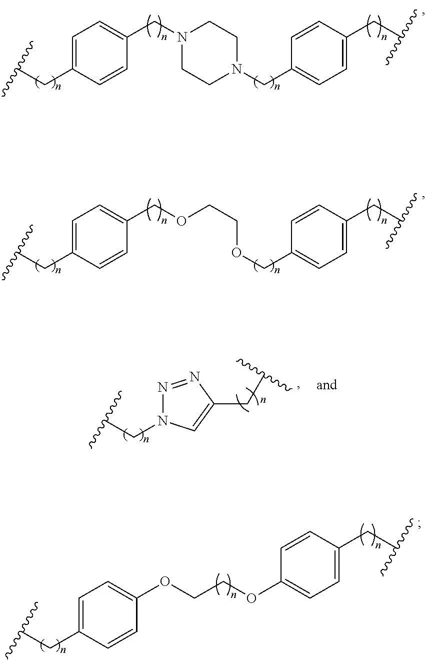

wherein n is independently selected at each occurrence from 1, 2, 3, 4, 5, and 6; and all other variables are as defined herein.

In some aspects, the linker moiety may comprise, either in whole or in part, a moiety selected from:

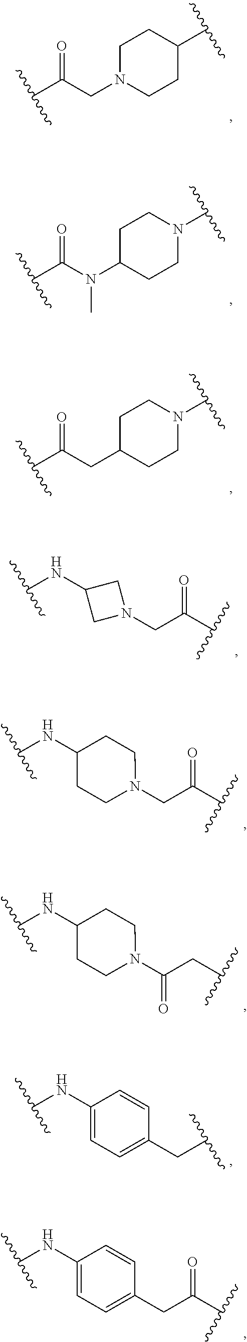

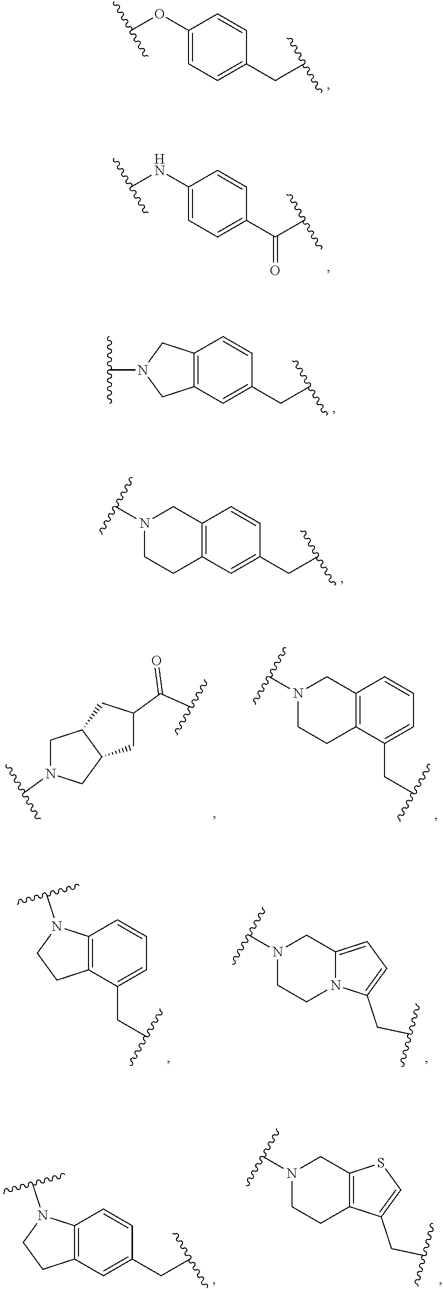

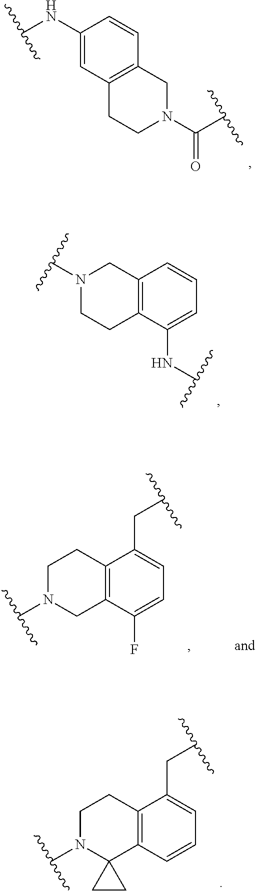 [SMILES: *Nc1ccc2c(c1)CCN(C(*)=O)C2 ; *Nc1cccc2c1CCN(*)C2 ; *Cc1ccc(F)c2c1CCN(*)C2 ; *Cc1cccc2c1CCN(*)C21CC1]

In some aspects, the linker moiety may comprise, either in whole or in part, a moiety selected from:

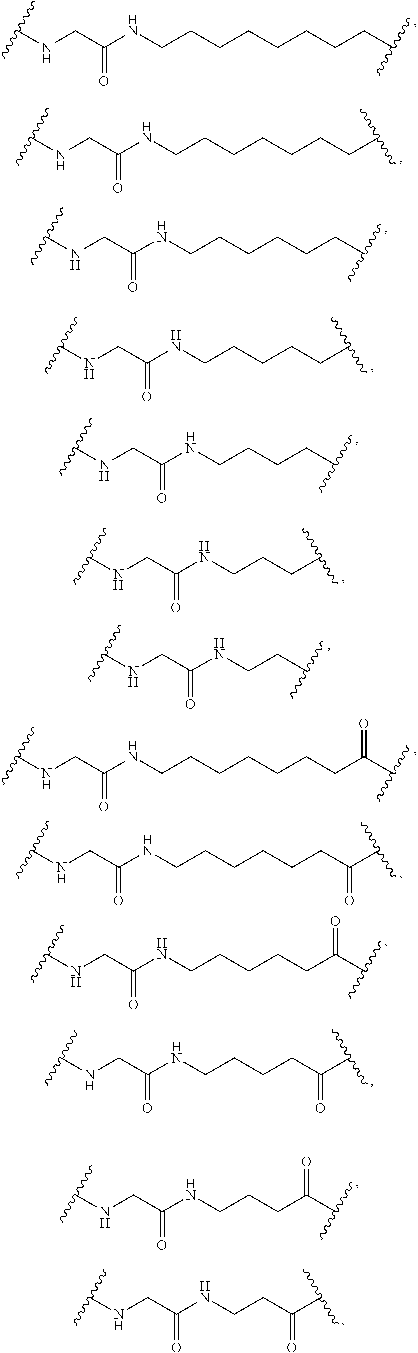

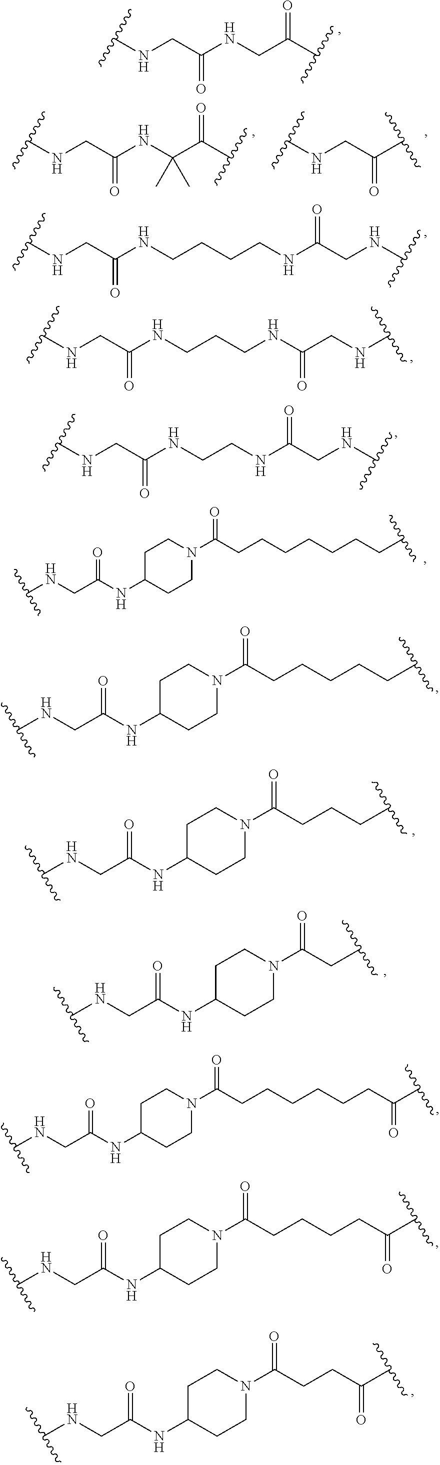

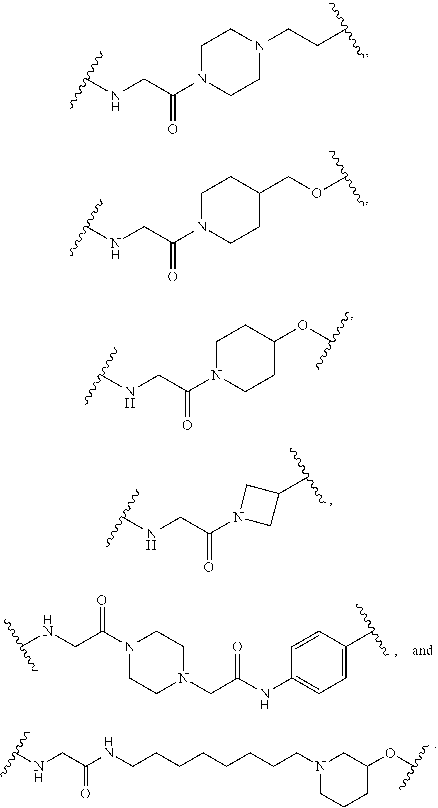 [SMILES: *CCN1CCN(C(=O)CN*)CC1 ; *NCC(=O)N1CCC(CO*)CC1 ; *NCC(=O)N1CCC(O*)CC1 ; *NCC(=O)N1CC(*)C1 ; *NCC(=O)N1CCN(CC(=O)Nc2ccc(*)cc2)CC1 ; *NCC(=O)NCCCCCCCCN1CCCC(O*)C1]

In some aspects, the linker moiety may comprise, either in whole or in part, a moiety selected from:

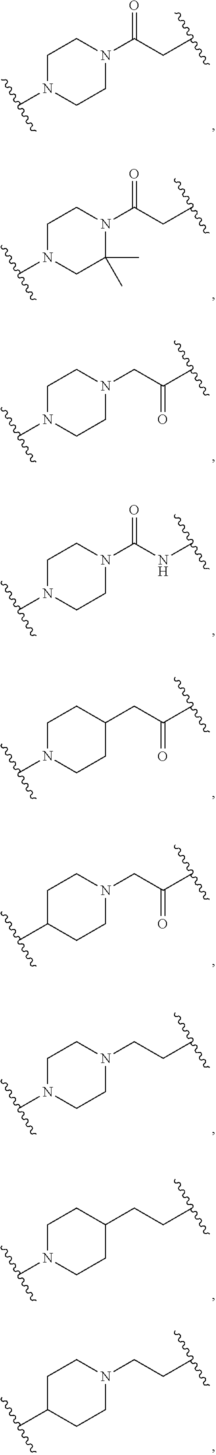

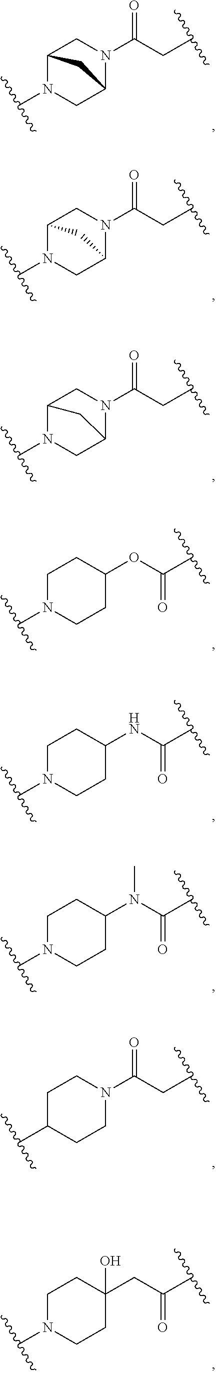

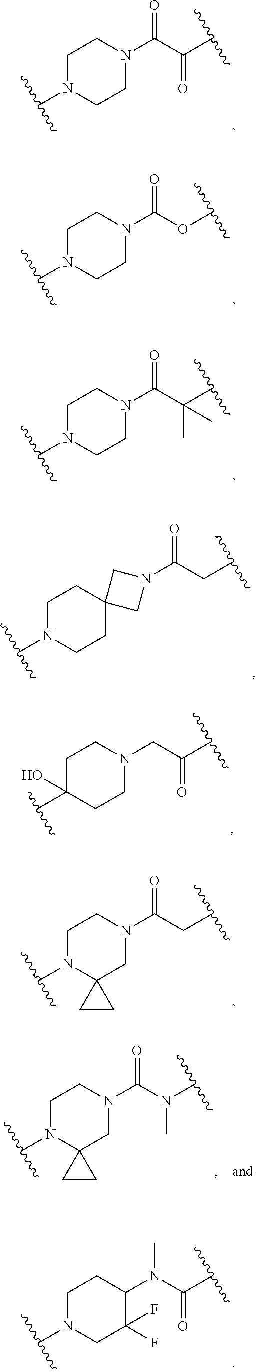

In some aspects, the linker moiety may comprise, either in whole or in part, a moiety selected from:

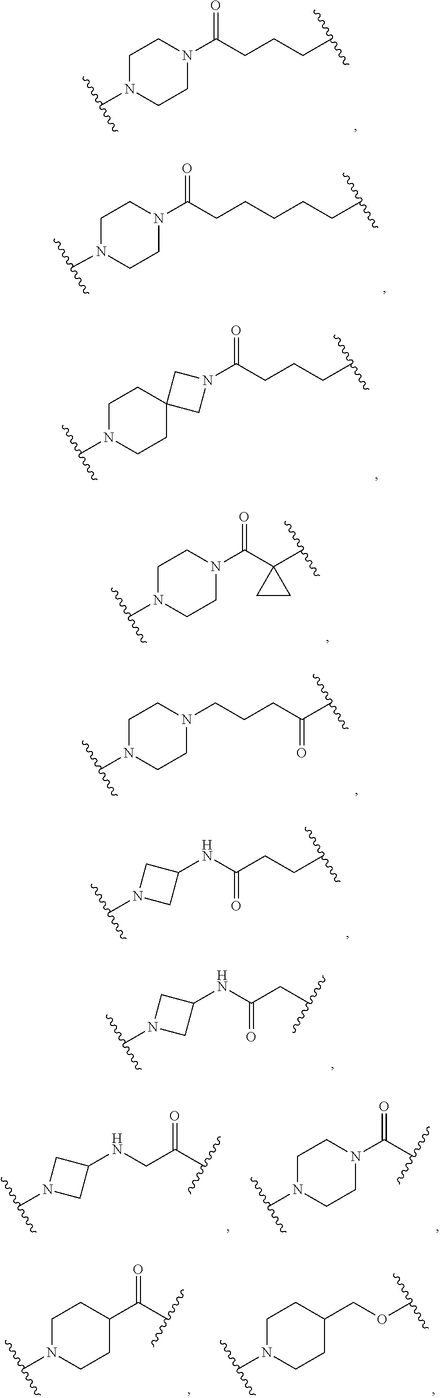

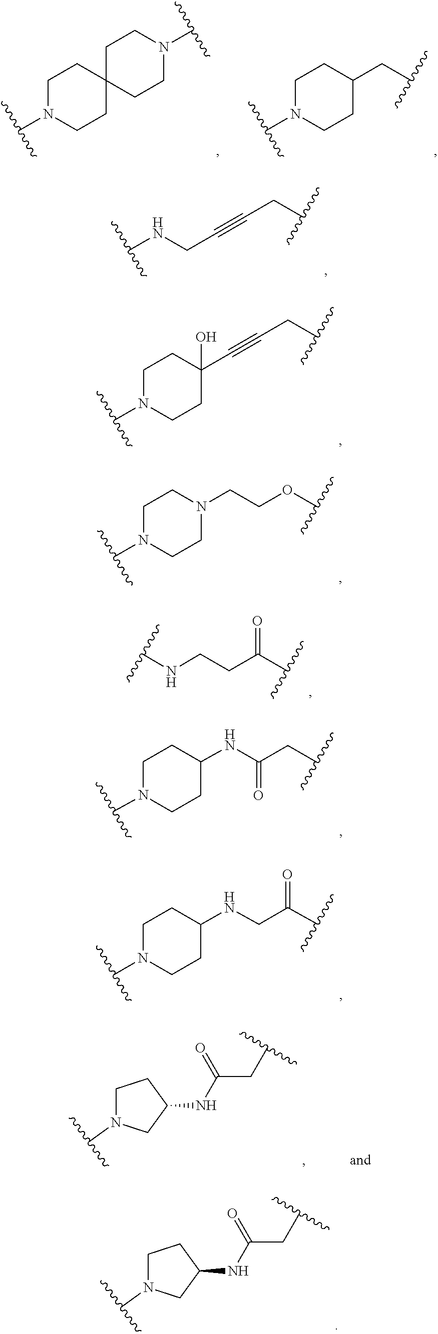

In some aspects, the linker moiety may comprise, either in whole or in part, a moiety selected from:

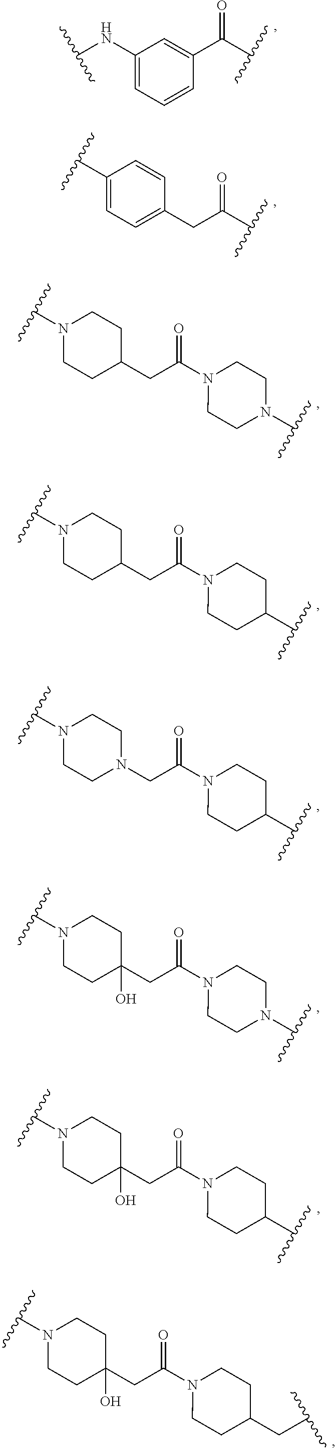

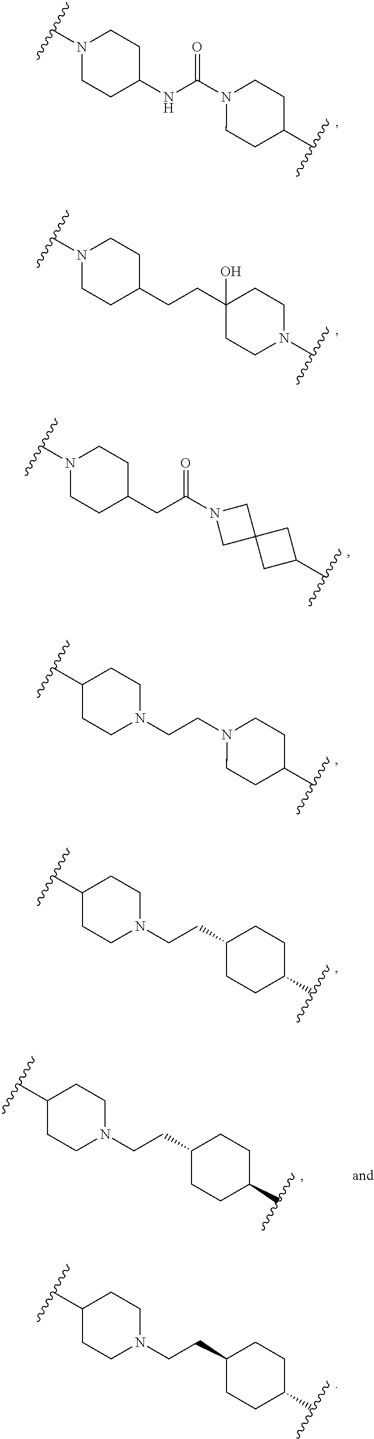

The binding moiety found in Formula I may be derived from any moiety identified as capable of binding to a target molecule of interest. In some aspects, the binding moiety is selected from a polypeptide, a ligand (e.g., a ligand for the target molecule of interest), an aptamer, a nanoparticle, and a small molecule.

In some aspects, when the binding moiety is a polypeptide, the moiety is an antibody. The terms “antibody” and “immunoglobulin” include antibodies and immunoglobulins of any isotope (e.g., IgG (e.g., IgG1, IgG2, IgG3, or IgG4), IgE, IgD, IgA, IgM, etc.), whole antibodies (e.g., antibodies composed of a tetramer which in term is composed of two dimers of a heavy and light chain polypeptide); single chain antibodies; fragments of antibodies (e.g., fragments of whole or single chain antibodies) which retain specific binding to the target molecule of interest, including, but not limited to, Fv, single chain Fv (scFv), Fab, F(ab′)₂, Fab′, (scFv′)₂, diabodies, and nanobodies; single domain antibodies (VHH); chimeric antibodies; monoclonal antibodies; fully human antibodies; humanized antibodies (e.g., humanized whole antibodies, humanized antibody fragments, etc.); and fusion proteins including an antigen-binding portion of an antibody and a non-antibody protein or fragment thereof. The antibody may be further conjugated to other moieties, such as, for example, polyethylene glycol (PEG), etc.

In some aspects, the binding moiety is a ligand for the target molecule of interest. As used herein, a “ligand” is a substance that forms a complex with a biomolecule to serve a biological purpose. The ligand may be a substance that forms a complex with the target molecule of interest. In some aspects, the ligand is modified in such a way that complex formation with the target molecule of interest occurs, but the normal biological result of such complex formation does not occur.

In some aspects, the binding moiety is an aptamer. By “aptamer” is meant a nucleic acid (e.g., an oligo nucleotide) that has a specific binding affinity for the target molecule of interest. Aptamers exhibit certain desirable properties, such as ease of selection and synthesis, high binding affinity and specificity, low immunogenicity, and versatile synthetic accessibility.

In some aspects, the binding moiety is a small molecule. By “small molecule” is meant a compound having a molecule weight of 1000 atomic mass units (amu) or less. In some aspects, the small molecule is 750 amu or less, 500 amu or less, 400 amu or less, 300 amu or less, or 200 amu or less. In some aspects, the small molecule is not made up of repeating molecular units, such as those present in a polymer.

In some aspects, the binding moiety specifically binds a target molecule of interest associated with a disease or disorder.

In some aspects, the binding moiety specifically binds a target molecule of interest associated with a cancer cell. By “cancer cell” is meant a cell exhibiting a neoplastic cellular phenotype, which may be characterized by one or more of, for example, abnormal cell growth, abnormal cellular proliferation, loss of density-dependent growth inhibition, anchorage-independent growth potential, ability to promote tumor growth or development in an immunocompromised non-human animal model, or any appropriate indicator of cellular transformation. “Cancer cell” may be used interchangeably herein with “tumor cell,” “malignant cell,” or “cancerous cell,” and encompasses cancer cells of a solid tumor, a semi-solid tumor, a hematological malignancy (e.g., a leukemia cell, a lymphoma cell, a myeloma cell, etc.), a primary tumor, a metastatic tumor, and the like.

In some aspects, the binding moiety specifically binds a target molecule of interest associated with an immune cell. In some aspects, the target molecule of interest is associated with an immune cell selected from a T cell, a B cell, a natural killer (NK) cell, a macrophage, a monocyte, a neutrophil, a dendritic cell, a mast cell, a basophil, and an eosinophil.

In some aspects, the binding moiety may be derived from a therapeutic agent that is capable of binding to the target molecule of interest or a derivative thereof. The term “therapeutic agent” includes any synthetic or naturally occurring biologically active compound or composition of matter which, when administered to an organism (either human or a nonhuman animal), induces a desired pharmacologic, immunogenic, and/or physiologic effect by local and/or systemic action. The term, therefore, encompasses those compounds or chemicals traditionally regarded as drugs, vaccines, and biopharmaceuticals, including molecules such as proteins, peptides, hormones, nucleic acids, gene constructs, and the like. Examples of therapeutic agents are described in well-known literature references such as the Merk Index (14^{th} Edition), the Physician's Desk Reference (64^{th} Edition), and The Pharmacological Basis of Therapeutics (12^{th} Edition), and they include, without limitation, medicaments; vitamins; mineral supplements, substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiandrenergics, antiarrhythmics, antihypertensive agents, hormones, and nutrients, antiarthritics, antiasthmatic agents, anticonvulsants, antihistamines, antinauseants, antineoplastics, antipruritics, antipyretics, antispasmodics, cardiovascular preparations (including calcium channel blockers, beta blockers, and beta-agonists), antihypertensives, diuretics, vasodilators, central nervous system stimulants, cough and cold preparations, decongestants, diagnostics, bone growth stimulants and bone resorption inhibitors, immunosuppressives, muscle relaxants, psychostimulants, sedatives, tranquilizers, proteins, peptides, and fragments thereof (whether naturally occurring, chemically synthesized or recombinantly produced), and nucleic acid molecules (polymeric forms of two or more nucleotides, either ribonucleotides (RNA) or deoxyribonucleotides (DNA) including both double and single-stranded molecules, gene constructs, expression vectors, antisense molecules and the like), small molecules and other biologically active macromolecules such as, for examples, proteins and enzymes. The agent may be a biologically active agent used in medical, including veterinary applications and in agriculture, such as with plants, as well as other areas.

In some exemplary aspects, the binding moiety may be derived from an anti-cancer agent. In some aspects, the binding moiety may be derived from a chemotherapeutic agent, for example but not limited to, azacytidine, capecitabine, carmofur, cladribine, clofarabine, cytarabine, decitabine, floxuridine, fludarabine, fluorouracil, gemcitabine, mercaptopurine, melarabine, pentostatin, tegafur, tioguanine, methotrexate, pemetrexed, raltitrexed, hydroxycarbamide, irinotecan, topotecan, daunorubicin, doxorubicin, epirubicin, idarubicin, mitoxantrone, valrubicin, etoposide, teniposide, cabazitaxel, docetaxel, paclitaxel, vinblastine, vincristine, vindesine, vinflunine, vinorelbine, bendamustine, busulfan, carmustine, chlorambucil, chlormethine, cyclophosphamide, dacarbazine, fotemustine, ifosfamide, lomustine, melphalan, streptozotocin, temozolomide, carboplatin, cisplatin, nedaplatin, oxaliplatin, altretamine, bleomycin, bortezomib, dactinomycin, estramustine, ixabepilone, mitomycin, procarbazine, afatanib, aflibercept, axitinib, bosutinib, crizotinib, dasatinib, erlotinib, gefitinib, imatinib, lapatinib, nilotinib, pazopanib, ponatinib, regorafenib, ruxotinib, sorafenib, sunitinib, vandetanib, everolimus, temsirolimus, alitretinoin, bexarotene, isotretinoin, tamibarotene, tretinoin, lenalidomide, pomalidomide, thalidomide, Panobinostat, romidepsin, valproate, vorinostat, anagrelide, and vemurafenib. In some aspects, PBM may be derived from a targeted cancer therapy, for example, imatinib, defitinib, erlotinib, sorafenib, sunitinib, dasatinib, lapatinib, nilotinib, bortezomib, tamoxifen, FGFR inhibitors (e.g. lucitanib, AZD4547, and pemigatinib), Janus kinase inhibitors (e.g., tofacitinib), ALK inhibitors (e.g., crizotinib), Bcl-2 inhibitors (e.g., venetoclax, obatoclax, navitoclax, and gossypol), PARP inhibitors, (e.g., olaparib, rucaparib, niraparib, and talazoparib), PI3K inhibitors (e.g., perifosine), apatanib, Braf inhibitors (e.g., vemurafenib, dabrafenib, LGX818), MEK inhibitors (e.g., trametinib, MEK162), CDK inhibitors (e.g., PD-0332991, LEE011), Hsp90 inhibitors, hedgehog pathway inhibitors (e.g., vismodegib or sonidegib), salinomycin, temsirolimus, everolimus, vemurafenib, trametinib, and dabrafenib. Other anti-cancer therapeutics from which PBM may be derived include afatinib, brigatinib, dacomitinib, erlotinib, gefitinib, icotinib, mobocertinib, olmutinib, Osimertinib, rociletinib, vandetanib, lapatinib, neratinib, tucatinib, avapritinib, axitinib, masitinib, pazopanib, ripretinib, sorafenib, sunitinib, toceranib, lestaurtinib, gilteritinib, axitinib, cediranib, lenvatinib, nintedanib, pazopanib, regorafenib, semaxanib, sorafenib, sunitinib, tivozanib, toceranib, vandetanib, alectinib, brigatinib, ceritinib, entrectinib, larotrectinib, infigratinib, pemigatinib, pralsetinib, selpercatinib, vandetanib, cabozantinib, capmatinib, crizotinib, asciminib, bosutinib, dasatinib, imatinib, nilotinib, panotinib, radotinib, baracitinib, fedratinib, filgotinib, lestaurtinib, momelotinib, pacritinib, ruxolitinib, binimetinib, cobimetinib, selumetinib, trametinib, crizotinib, entrectinib, lorlatinib, acalaburitnib, ibrutinib, zanubrutinib, aflibercept, everolimus, ridaforolimus, temsirolimus, glasdegib, sonidegib, vismodegib, abemaciclib, palbociclib, ribociclib, trilaciclib, cabozantinib, capmatinib, entrectinib, erdafitinib, gilteritinib, larotrectinib, Lenvatinib, masitinib, midostaurin, nintedanib, pazopanib, pemigatinib, pexidartinib, quizartinib, regoragenib, ripretanib, sorafenib, sotorasib, sunitinib, tepotinib, vandetanib, and venetoclax.

In some aspects, the target molecule may comprise a target protein. “Target protein” is used herein to describe a protein or polypeptide, which is the target for binding to the compounds according to the present disclosure. Target proteins may include any protein or peptide that may be bound by the compounds described herein, including fragments thereof, analogs thereof, and/or homologs thereof. Target proteins include proteins or peptides having any biological functional or activity, including structural, regulatory, hormonal, enzymatic, genetic, immunological, contractile, storage, transportation, and signal transduction. The target protein may include, in some aspects, structural proteins, receptors, enzymes, cell surface proteins, proteins pertinent to the integrated function of a cell, including proteins involved in catalytic activity, aromatase activity, motor activity, helicase activity, metabolic processes (anabolism and catabolism), antioxidant activity, proteolysis, biosynthesis, proteins with kinase activity, oxidoreductase activity, transferase activity, hydrolase activity, lyase activity, isomerase activity, ligase activity, enzyme regulatory activity, signal transducer activity, structural molecule activity, binding activity (for protein, lipid, or carbohydrate), receptor activity, cell motility, membrane fusion, cell communication, regulation of biological processes, development, cell differentiation, response to stimulus, behavioral proteins, cell adhesion proteins, proteins involved in cell death, proteins involved in transport including protein transporter activity, nuclear transport, iron transporter activity, channel transporter activity, carrier activity, permease activity, secretion activity, electron transporter activity, pathogenesis, chaperone regulator activity, nucleic acid binding activity, transcription regulator activity, extracellular organization and biogenesis activity, or translation regulator activity. Target proteins of interest can include proteins from eukaryotes and prokaryotes, including microbes, viruses, fungi, and parasites, including humans, microbes, viruses, fungi, insects, and parasites, among numerous others, including other animals, including mice, rats, monkeys, domesticated animals, microbes, plants, and viruses.

The target protein may be endogenous or non-endogenous to the cell. In some aspects, the target protein is an endogenous protein. In some aspects, the target protein is an endogenous protein that mediates a disorder. The endogenous protein can be the normal form of the protein or an aberrant form. In some aspects, the target protein may be a mutant form, fusion form, or truncated form of an endogenous protein associated with a specific disorder or condition, for example, cancer, which may be, for example, a partial or full gain-of-function or loss-of-function mutant encoded by nucleotide polymorphisms. In some aspects, the compounds specifically targets an aberrant form of the target protein and not a normal form. In some aspects, the target protein may be a non-endogenous protein, such as from a pathogen or toxin. In some aspects, the target protein is a non-endogenous protein from a virus, for example, HIV, HBV, HCV, RSV, HPV, CMV, flavivirus, pestivirus, coronavirus, norovirus, etc. In some aspects, the target protein is a non-endogenous protein from a bacteria, for example, a gram-positive or gram-negative bacteria or mycobacteria. In some aspects, the target protein is a non-endogenous protein from a fungus. In some aspects, the target protein is a non-endogenous protein from a prion. In some aspects, the target protein is a non-endogenous protein derived from a eukaryotic pathogen, such as a protist, helminth, etc.

Representative examples of target protein include, but are not limited to, retinoid X receptor (RXR), dihydrofolate reductase (DHFR), heat shock protein 90 (HSP90), tyrosine kinase, serine/threonine kinase, aurora kinase, ATM, ATR, BPTF, ALK, ABL, JAK2, MET, mTORC1, mTORC2, Mast/stem cell growth factor receptor (SCFR), IGF1R, HDM2, MDM2, HDAC, RAF receptor, nuclear receptor, epigenetic modulatory protein, androgen receptor, estrogen receptor, thyroid hormone receptor, HIV protease, HIV integrase, AP1, AP2, MCL-1, DNA-PK, eIF4E, IDH1, RAS, RASK, MERTK, MER, EGFR, FLT3, SMARCA2, CDK9, CDK12, CDK13, glucocorticoid receptor, RasG12C, Her3, Bcl-2, Bcl-XL, PPAR-gamma, BCR-ABL, BRAF, LRRK2, PDGFRα, RET, fatty acid binding protein, FLAP, Kringle Domain V 4BVV, lactoylglutathione lyase, mPGES-1, Factor Xa, Kallikrein 7, Cathepsin K, Cathepsin L, Cathepsin S, MTH1, MDM4, PARP1, PARP2, PARP3, PARP14, PARP15, PDZ domain, phospholipase A2 domain, protein S100-A7 2WOS, NRASQ6IK, NRASQ61R, TEAD1, TEAD2, TEAD3, TEAD4, Saposin-B, Sec7, pp60 SrC, Tank1, Ubc9 SUMO E2 ligase SF6D, Src, Src-AS1, Src-AS2, JAK3, MEK1, KIT, KSR1, CTNNB1, BCL6, PAK1, PAK4, TNIK, MEN1, ERK1, IDO1, CBP, ASH1L, ATAD2, YAP, BAZ2A, BAZ2B, BDRT, BDR9, SMARCA4, PB1, TRIM24, TIF1a, BRPF1, CECR2, CREBBP, PCAF, PHIP, TAF1, HDAC2, HDAC4, HDAC6, HDAC7, HDAC8, KAT2B, WWTR1, A2aR, alpha-subunit of FTase and/or GGTase, ARGI, B-TrCP, CBX7, Cdc7/ASK, Cdc7-Dbf4, KAT2A, HAT1, ATF2, KAT5, KDM1A, DOT1L, EHMT1, ceacam-1, CENP-E, clAP1/2, DKC1, DMT3A, DNA replication/repair protein, DNA2, DNMT3B, E2F1, EFHD2/SWIPROSIN, Eg5, EMI1, ERCCD1/XPF, EWS-FLI, FoxA1, GATA3, FOXP1, GCN2, GNAQ, GNA11, SETD2, SETD5, SETD8, SETDB1, SMYD2, SMYD3, SUV4-20H1, ErbB2 receptor, ErbB4 receptor, VEGFR1 receptor, VEGFR2 receptor, VEGFR3 receptor, PDGFRP receptor, Lyn receptor, Hck receptor, c-MET receptor, TrkB receptor, Axl receptor, YES receptor, HER2, PNET receptor, RCC receptor, RAMP receptor, SEGA receptor, PDGFR receptors, ErbB2 receptor, HK2, HSP70, IAPs, IQGAPI, LSF, MCT1, MCT4, MEF2B, MMP3, MMP14, MUC1, MyB, Myd88, FGFR1 receptor, FGFR2 receptor, FGFR3 receptor, FGFR4 receptor, PDGRF receptor, DDR1 receptor, PDGRα receptor, PDGRβ receptor, CDK4 receptor, CDK6 receptor, Fins receptor, T3151 VEGFR receptor, FGFR receptor, Flt 3 receptor, Eph2A receptor, JAK1 receptor, FKBP12 receptor, mTOR receptor, CDK 8 receptor, CDF-1R receptor, MEK2 receptor, Brk receptor, PI3Ka receptor, GCN5 receptor, G9a, EHMT2, EZH2, EED, PRMT3, PRMT4, PRMT5, PRMT6, NR2F6, NSD1, P70S6K, PIN1, SERCA, SF3B1, Sirtuin 2, Skp2, SMAD3, SPOP, Tall, KDM1, KDM4, KDM5, KDM6, L3MBTL3, Menin, HDAC6, HDAC7, PTP1B, SHP2, TBK1, Trib2, TRIF, TS, XPO1, RASN, ARIF1B Scavenger mRNA-decapping enzyme DcpS, ALK, BTK, NTRK1, NTRK2, NTRK3, IDO, ERK2, ABL1, ABL2, ATK1, ATK2, BMX, CSK, EPHA3, EPHA4, EPHA7, EPHB4, FES, FYN, GSG2, ISNR, HBV, CBL-B, ERK, WDR5, NSP3, IRAK4, NRAS, ADAR, ASCL1, PAX8, TP63, SARM1, Ataxin-2, KSR2, CSCR4, HDAC10, NSD2, WHSC1, RIT1, WRN, BAPI, EPASI, HIF2α, GRB2, KMT2D, MLL2, MLL4, MLLT1, ENL, NSD3, PPM1D, WIP1, SOS1, TBXT, Brachyury, USP7, BKV, JCV, CK1α, GSPT1, ERF3, IFZV, TAU, CYP17A1, SALL4, FAM38, CYP20A1, HTT, NRF2, NFE2L2, P300, PIK3CA, SARM1, SNCA, MAPT, TCPTP, STAT3, MyD88, PTP4A3, SF3B1, ARID1B, and ARID2.

In some aspects, the target protein may comprise or be derived from a tyrosine kinase (e.g., AATK, ABL, ABL2, ALK, AXL, BLK, BMX, BTK, CSF1R, CSK, DDR1, DDR2, EGFR, EPHA1, EPHA2, EPHA3, EPHA4, EPHA5, EPHA6, EPHA7, EPHA8, EPHA10, EPHB1, EPHB2, EPHB3, EPHB4, EPHB6, ERBB2, ERBB3, ERBB4, FER, FES<FGFR1, FGFR2, FGFR3, FGFR4, JAK1, JAK2, JAK3, KDR, KIT, KSR1, LCK1, LMTK2, LMTK3, LTK, LYN, MATK, MERTK, MET, MLTK, MST1R, MUSK, NKR1, NTRK1, NTRK2, NTRK3, PDGFRA, PDGFRB, PLK4, PTK2, PTK2B, PTK6, PTK7, RET, ROR1, ROR2, ROS1, RYK, SGK493, SRC, SRMS, STYK1, SYK, TEC, TEK, TEX14, TIE1, TNK1, TINK2, TNNI3K, TXK, TYK2, TYRO3, YES1, or ZAP70).

In some aspects, the target protein may comprise or be derived from a serine/threonine kinase (e.g., casein kinase 2, protein kinase A, protein kinase B, protein kinase C, Raf kinases, CaM kinases, AKT1, AKT2, AKT3, ALK1, ALK2, ALK3, ALK4, Aurora A, Aurora B, Aurora C, CHK1, CHK2, CLK1, CLk2, CLK3, DAPK1, DAP2, DAPK3, DMPK, ERK1, ERK2, ERK5, GCK, GSK3, HIPK, KHS1, LKB1, LOK, MAPKAPK2, MAPKAPK, MNK1, MSSK1, MST1, MST2, MST4, NDR, NEK2, NEK3, NEK6, NEK7, NEK9, NEK11, PAK1, PAK2, PAK3, PAK4, PAK5, PAK6, PIM1, PIM2, PLK1, RIP2, RIP5, RSK1, RSK2, SGK2, SGK3, SIK1, STK33, TAOT, TAO2, TGF-beta, TLK2, TSSK1, TSSK2, ULK1, or ULK2).

In some aspects, the target protein may comprise or be derived from a cyclin-dependent kinase, for example, CDK1, CDK2, CDK3, CDK4, CDK5, CDK6, CDK7, CDK8, CDK9, CDK10, CDK11, CDK12, or CDK13.

In some aspects, the target protein may comprise or be derived from a leucine-rich repeat kinase (e.g., LRRK2).

In some aspects, the target protein may comprise or be derived from a lipid kinase (e.g., PIK3CA, PIK3CB) or a sphingosine kinase (e.g., SIP).

In some aspects, the target protein may comprise or be derived from a nuclear protein, for example BRD1, BRD2, BRD3, BRD4, and other epigenetic proteins, antennapedia homeodomain protein, BRCA1, BRCA2, CCAAT-Enhanced-Binding proteins, histones, polycomb-group proteins, high mobility group proteins, telomere binding proteins, FANCA, FANCD2, FANCE, FANCF, hepatocyte nuclear factors, Mad2, NF-kappa B, nuclear receptor coactivators, CREB-binding protein, p55, p107, p130, Rb proteins, p53, c-fos, c-jun, c-mdm2, c-myc, and c-rel.

In some aspects, the binding moiety may be selected or derived from such suitable moieties as cytokines, hormones, growth factors, neurotransmitters, adhesion molecules, and the like. Binding moieties can also include any molecules produced, used, or recognized in carbohydrate metabolism, energy metabolism, fatty acid and lipid metabolism, nucleotide metabolism, amino acid metabolism, and co-factor and Vitamin metabolism. (For a current listing of metabolic pathways and metabolites, see Boehringer Mannheim Biochemical Chart, available on the internet) (See also Salway (1999) Metabolism at Glance, Blackwell Science Inc.; 2nd ed.). This includes, but is not limited to, molecules such as carbohydrates (e.g., glucose, galactose, mannose, glycosaminoglycans, etc.), organic acids (e.g., lactate, citrate, tartrate, acetate, etc.), amino acids (e.g., methionine, tyrosine, glutamate, taurine, omithine, glutathione, etc.), halides (e.g., iodine, iodotyrosines chlorine, fluorine), Steroids (e.g., estrogen, progesterone, testosterone, etc.), fatty acids (e.g., glycerol, palmitate, Stearate, oleate, myrisates, etc.), lipids (e.g., cholesterol, phosphatidyl choline, ceramide, gangliosides, phospholipids such as anionic phospholipids, etc.), Vitamins (e.g., thiamine, folate, biotin, riboflavin, niacin, etc.), nucleic acids and derivatives thereof (e.g., ATP AMP, GTP, GMP, thiouracil, thymidine, urate, hypoxanthine, etc.), polypeptides and derivatives thereof, neurotransmitters (e.g., dopamine, serotonin, epinephrine, etc.), inorganic molecules (e.g., pyrophosphate, phosphate, phosphonates, Sulfates, etc.), and drugs with proven action (e.g., therapeutic compounds).

By “specifically binds” is meant that the binding moiety binds to its target with greater affinity, avidity, more readily, and/or with greater duration than they bind to other substances, e.g., in a sample. In some aspects, the binding moiety binds to its target with an affinity of Kₐ (that is, an equilibrium association constant of a particular binding interaction with units of 1/M) of, for example, greater than or equal to about 10⁵ M⁻¹. In some aspects, the binding moiety binds to its target with a Kₐ greater than or equal to 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding refers to a binding with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater. Alternatively, affinity may be defined as an equilibrium dissociation constant (K_{D}) of a particular binding interaction with units of M (e.g., 10⁻⁵ M to 10⁻¹³ M, or less). In some aspects, specific binding means the binding moiety binds to its target with a K_{D} of less than or equal to about 10⁻⁵ M, less than or equal to about 10⁻⁶ M, less than or equal to about 10⁻⁹ M, 10⁻¹⁰ M, 10⁻¹M or 10⁻¹² M or less. The binding affinity of the binding moiety to its target can be readily determined using conventional techniques, e.g., by competitive ELISA (enzyme-linked immunosorbent assay), equilibrium dialysis, by using surface plasmon resonance (SPR) technology, radioimmunoassay, or the like.

### Methods of Use

In another aspect, methods of making and using the compounds described herein are also provided.

In one aspect, a method is provided of fluorescently labeling a molecule, wherein the molecule comprises one or more nucleophilic moieties, the method comprising reacting the molecule with a compound selected from:

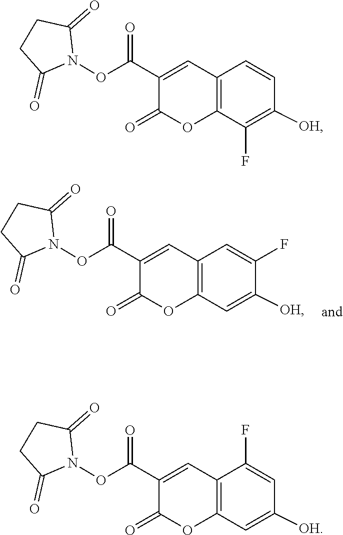 [SMILES: O=C(ON1C(=O)CCC1=O)c1cc2ccc(O)c(F)c2oc1=O ; O=C(ON1C(=O)CCC1=O)c1cc2cc(F)c(O)cc2oc1=O ; O=C(ON1C(=O)CCC1=O)c1cc2c(F)cc(O)cc2oc1=O]

In some aspects, the one or more nucleophilic moieties comprise one or more amino groups. In some aspects, the molecule is selected from a protein, a peptide, an antibody, an antigen, a receptor, a nucleic acid, a nucleotide, a nucleotide derivative, a therapeutic agent, a small molecule, a synthetic oligomer, a synthetic polymer, a hormone, a lymphokine, a cytokine, a toxin, a ligand, a carbohydrate, a sugar, an oligosaccharide, a polysaccharide, a fatty acid, or the like.

In some aspects, the molecule comprises a molecule of Formula I-A:

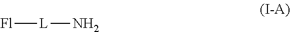

wherein Fl and L are as defined herein.

In some aspects, a method of preparing a molecule of Formula I

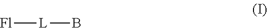

the method comprising:
reacting a compound of Formula I-A

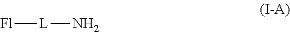

with a compound selected from

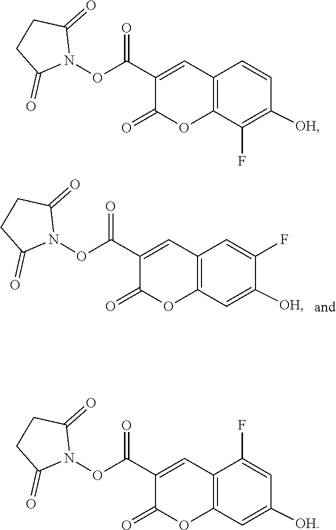 [SMILES: O=C(ON1C(=O)CCC1=O)c1cc2ccc(O)c(F)c2oc1=O ; O=C(ON1C(=O)CCC1=O)c1cc2cc(F)c(O)cc2oc1=O ; O=C(ON1C(=O)CCC1=O)c1cc2c(F)cc(O)cc2oc1=O]

to provide the compound of Formula I,
wherein Fl, L, and B are as defined herein.

The compounds disclosed herein can be used in imaging in vitro or in vivo using any suitable system according to the general principals of fluorescence, optical image acquisition, and/or image processing. In some aspects, a method of imaging a sample is provided. In some aspects, the method comprises contacting the sample with a compound comprising one or more fluorescent moieties selected from:

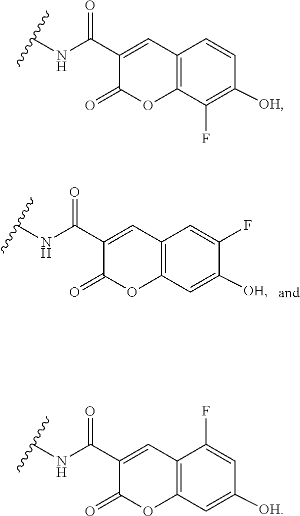 [SMILES: *NC(=O)c1cc2ccc(O)c(F)c2oc1=O ; *NC(=O)c1cc2cc(F)c(O)cc2oc1=O ; *NC(=O)c1cc2c(F)cc(O)cc2oc1=O]

In some aspects, the method further comprises exposing the sample to light of a wavelength that excites the one or more fluorescent moieties of the compound. In some aspects, the method further comprises detecting an optical signal emitted by the compound. In some aspects, the method further comprises after contacting the sample, allowing the compound to localize at a target within the sample prior to exposing the sample to the light. In some aspects, the compound localizes by binding to the target. In some aspects, the optical signal emitted by the compound confirms the presence of the target within the sample. In some aspects, the sample comprises a biomolecule, a cell, cell cultures, a cell lysate, or a tissue sample.

In another aspect, a method of imaging a subject is provided. In some aspects, the method comprises administering to the subject a compound comprising one or more fluorescent moieties selected from:

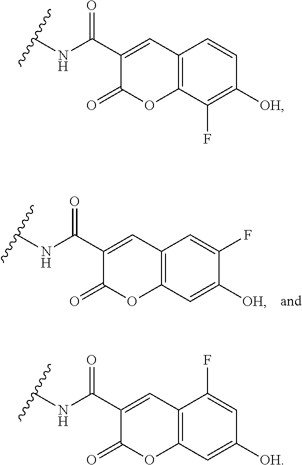 [SMILES: *NC(=O)c1cc2ccc(O)c(F)c2oc1=O ; *NC(=O)c1cc2cc(F)c(O)cc2oc1=O ; *NC(=O)c1cc2c(F)cc(O)cc2oc1=O]

wherein upon administration the compound localizes to one or more target sites.

In some aspects, the method further comprises exposing the one or more target sites to light of a wavelength that excites the one or more fluorescent moieties of the compound. In some aspects, the method further comprises detecting an optical signal emitted by the compound. In some aspects, the subject is a mammal, such as a rat, mouse, or human. In some aspects, the compound localizes to one or more target sites by binding to a target molecule present in the one or more target sites. In some aspects, the optical signal emitted by the one or more fluorescent moieties confirms the presence of the target molecule within the one or more target sites.

In another aspect, a method of detecting the presence of a target molecule in a sample is provided. In some aspects, the method comprises contacting the sample with a compound of Formula I, as described herein. In some aspects, the method further comprises exposing the sample to light of a wavelength that excites Fl of the compound of Formula I. In some aspects, the method further comprises detecting an optical signal emitted by the compound of Formula I, wherein the optical signal confirms the presence of the target molecule within the sample. In some aspects, the sample comprises a cell, cell culture, cell lysate, or a tissue sample. In some aspects, the compound of Formula I binds to the target molecule.

In another aspect, a method of detecting the presence of a target molecule in one or more target sites of a subject is provided. In some aspects, the method comprises administering to the subject a compound of Formula I as described herein, wherein the compound of Formula I localizes to the one or more target sites comprising the target molecule. In some aspects, the method further comprises exposing the one or more target sites to light of a wavelength that excites Fl of the compound of Formula I. In some aspects, the method further comprises detecting an optical signal emitted by the compound of Formula I, wherein the optical signal confirms the presence of the target molecule in the one or more target sites. In some aspects, the subject is a mammal, such as a rat, mouse, or human. In some aspects, the compound of Formula I binds to the target molecule. In some aspects, the target molecule is associated with the presence, absence, or state of a disease. In some aspects, the target molecule is associated with a physiological or metabolic state of the subject.

In another aspect, a method is provided for determining binding affinity between a target molecule and a test compound. In some aspects, the method comprises contacting the target molecule with a compound of Formula I, wherein the compound interacts with the target molecule. In some aspects, the method further comprises measuring a first optical signal from the compound of Formula I. In some aspects, the method further comprises introducing the test compound. In some aspects, the method further comprises measuring a second optical signal from the compound. In some aspects, the method further comprises calculating a difference in the second optical signal and the first optical signal, thereby determining the binding affinity between the target molecule and the test compound. In some aspects, the target molecule comprises a target protein, as described herein. In some aspects, the method is performed in a cell. The first optical signal, the second optical signal, or both may be measured by any suitable method as known in the art. For example, the first optical signal, the second optical signal, or both may be measured via fluorescence microscopy, including confocal microscopy, or flow cytometry. In some aspects, the first optical signal, the second optical signal, or both are provided upon exposing the sample to light of a wavelength that excites Fl of the compound.

In some aspects, the disclosed methods find use in drug discovery, drug validation, drug target discovery, high throughput screening, high content screening, drug development, or drug target validation. In certain aspects, the binding interaction between a test compound (e.g., a drug-like small molecule) and a target molecule can be detected, validated, and/or characterized. In some aspects, the relative binding affinity of test compounds for a target molecule can be determined by their ability to displace the compound of Formula I. Specifically, higher binding affinity of a first test compound relative to a second test compound is indicated by requiring a lower concentration of the first test compound to displace the compound of Formula I relative to the second test compound. Displacement of the compound of Formula I is determined by the loss or reduction of fluorescence from the compound of Formula I. In some aspects, the concentration of the test compound needed to displace the compound of Formula I is used to estimate binding (e.g., EC₅₀, IC₅₀) or the inhibitory constant (Kᵢ) for the test compound. In some aspects, the development of new or modified compounds is guided by their ability to displace the compound of Formula I from the target protein. This can be used to measure the selectivity of engagement of specific target proteins by small molecules.

In some aspects, a collection of test compounds that may have unknown binding affinity to a target molecule may be screened for their ability to bind the target molecule by determining their ability to displace the compound of Formula I. In some aspects, test compounds may be screened for their ability to bind to a first target molecule preferentially and/or relatively to a second target molecule by their ability to displace the compound of Formula I from the first target molecule relative to the compound of Formula I from the second target molecule.

The disclosed steps in any of the above methods can also be repeated at predetermined intervals, thereby allowing for the evaluation of the optical signal of the compound in a sample or subject over time. In some aspects, the emitted signal may take the form of an image. In some aspects, the subject may be a mammal, including a human, but may also be non-mammalian (e.g., C. elegans, Drosophila, Spodoptera frugiperda, etc.). The samples can include, without limitation, cells, cell cultures, tissue sections, cytospin samples, and the like.

In another aspect, kits are provided comprising any of the compounds or compositions described herein and for carrying out any of the methods described herein. In some aspects, the kits comprise, in separate or the same contains, one or more compounds as described herein or individual elements for making said compounds. The kits may also optionally include buffers, biological materials such as nucleic acids, proteins, and/or cells, or containers as well as written instructions for carrying out the methods described herein, such as instructions regarding the reconstitution of reagents, the application of the reagents to samples or subjects (e.g., by treatment of cells or injection into a live animal) and/or regarding imaging, detection, validation, or normalization protocols. In the case of prepackaged compounds, the kits optionally include pre-measured and pre-dosed compounds that are ready to incorporate into the methods without measurement, e.g., premeasured fluid aliquots or pre-weighed or pre-measured solid reagents that may be easily reconstituted by the end-user of the kit.

In view of the described compounds, compositions, and methods, hereinbelow are described certain more particular aspects of the disclosure. These particularly recited aspects should not, however, be interpreted to have any limiting effect on any different claims containing different or more general teachings described herein or that the “particular” aspects are somehow limited in some way other than the inherent meanings of the language and formulae literally used therein.

Aspect 1. A compound comprising one or more fluorescent moieties selected from:

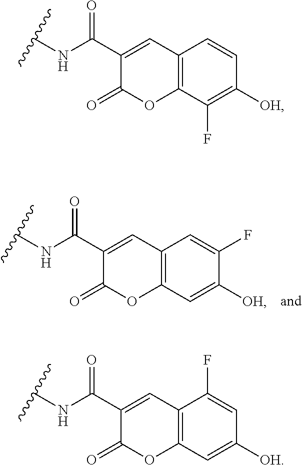 [SMILES: *NC(=O)c1cc2ccc(O)c(F)c2oc1=O ; *NC(=O)c1cc2cc(F)c(O)cc2oc1=O ; *NC(=O)c1cc2c(F)cc(O)cc2oc1=O]

Aspect 2. The compound of aspect 1, wherein the compound comprises a protein, a peptide, an antibody, an antigen, a receptor, a nucleic acid, a nucleotide, a nucleotide derivative, a therapeutic agent, a small molecule, a synthetic oligomer, a synthetic polymer, a hormone, a lymphokine, a cytokine, a toxin, a ligand, a carbohydrate, a sugar, an oligosaccharide, a polysaccharide, or a fatty acid.

Aspect 3. The compound of aspect 1, wherein the compound comprises a fluorescent probe.

Aspect 4. A compound of Formula I

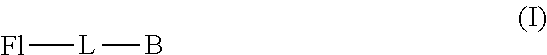

wherein:
Fl is selected from:

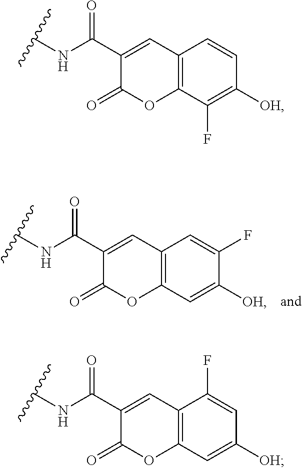 [SMILES: *NC(=O)c1cc2ccc(O)c(F)c2oc1=O ; *NC(=O)c1cc2cc(F)c(O)cc2oc1=O ; *NC(=O)c1cc2c(F)cc(O)cc2oc1=O]

L is selected from a bond or a linker moiety; and
B is a binding moiety.

Aspect 5. The compound of aspect 4, wherein Fl is

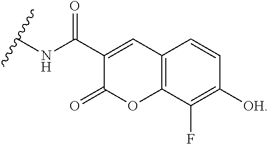 [SMILES: *NC(=O)c1cc2ccc(O)c(F)c2oc1=O]

Aspect 6. The compound of aspect 4, wherein Fl is

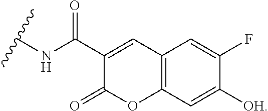 [SMILES: *NC(=O)c1cc2cc(F)c(O)cc2oc1=O]

Aspect 7. The compound of aspect 4, wherein Fl is

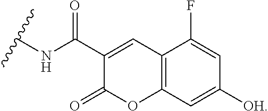 [SMILES: *NC(=O)c1cc2c(F)cc(O)cc2oc1=O]

Aspect S. The compound of any one of aspects 4-7, wherein L is a bond.

Aspect 9. The compound of any one of aspects 4-7, wherein L is a linker moiety.

Aspect 10. The compound of any one of aspects 4-7, wherein L is a linker moiety selected from L1:

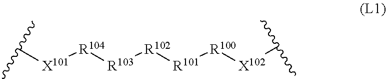

wherein:
   X¹⁰¹ and X¹⁰² are independently at each occurrence selected from a bond, aryl, heteroaryl, cycloalkyl, heterocycle, NR¹³⁰, C(R¹³⁰)₂, O, C(O), and S;
   R¹⁰⁰, R¹⁰¹, R¹⁰², R¹⁰³, and R¹⁰⁴ are independently at each occurrence selected from the group consisting of a bond, alkyl, —C(O)—, —C(O)O—, —OC(O)—, —SO₂—, —S(O)—, C(S)—, —C(O)NR¹³⁰—, —NR¹³⁰C(O)—, —O—, —S—, —NR¹³⁰—, —C(R¹³⁰R¹³⁰)—, —P(O)(OR¹⁰⁶))—, —R(O)(OR¹⁰⁶)—, alkenyl, alkynyl, haloalkyl, alkoxy, aryl, heterocycloalkyl, cycloalkyl, heteroaryl, lactic acid, or glycolic acid, each of which may be optionally substituted with one or more (for example, 1, 2, 3, or 4) substituents independently selected from R¹⁴⁰;
   R¹⁰⁶ is independently at each occurrence selected from the group consisting of hydrogen, alkyl, arylalkyl, heteroarylalkyl, alkenyl, alkynyl, aryl, heteroaryl, cycloalkyl, or heterocycloalkyl;
   R¹³⁰ is independently as each occurrence selected from the group consisting of hydrogen, alkyl, cycloalkyl, heterocycloalkyl, aryl, heteroaryl, —C(O)H, —C(O)OH, —C(O)alkyl, —C(O)Oalkyl, —C(O)(cycloalkyl, heterocycloalkyl, aryl, or heteroaryl), —C(O)O(cycloalkyl, heterocycloalkyl, aryl, or heteroaryl), alkenyl, or alkynyl; and
   R¹⁴⁰ is independently at each occurrence selected from the group consisting of hydrogen, alkyl, alkenyl, alkynyl, fluoro, bromo, chloro, hydroxyl, alkoxy, azide, amino, cyano, —NH(alkyl, cycloalkyl, heterocyloalkyl, aryl, or heteroaryl), —N(independently alkyl, cycloalkyl, heterocycloalkyl, aryl, or heteroaryl), —NHSO₂(alkyl, cycloalkyl, heterocycloalkyl, aryl, or heteroaryl), —N(alkyl, cycloalkyl, heterocycloalkyl, aryl, or heteroaryl)SO₂alkyl, —NHSO₂alkenyl, —N(alkyl)SO₂alkenyl, —NHSO₂alkynyl, —N(alkyl)SO₂alkynyl, haloalkyl, cycloalkyl, heterocycloalkyl, aryl, or heteroaryl.

Aspect 11. The compound of any one of aspects 4-7, wherein L is selected from:

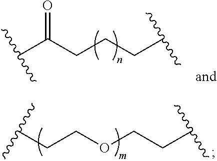

wherein:
n is an integer from 0 to 20; and
m is an integer from 0 to 5.

Aspect 12. The compound of any one of aspects 4-11, wherein the binding moiety is capable of binding to a target molecule.

Aspect 13. The compound of aspect 12, wherein the target molecule comprises a target protein.

Aspect 14. The compound of any one of aspects 4-13, wherein the binding moiety is selected from a polypeptide, a ligand, an aptamer, a nanoparticle, and a small molecule.

Aspect 15. The compound of any one of aspects 4-14, wherein the binding moiety comprises a therapeutic agent or a derivative thereof.

Aspect 16. The compound of aspect 15, wherein the binding moiety comprises or is derived from an anti-cancer agent.

Aspect 17. The compound of aspect 15 or aspect 16, wherein the binding moiety comprises taxol or dasatanib.

Aspect 18. A compound of the formula:

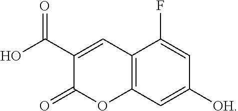 [SMILES: O=C(O)c1cc2c(F)cc(O)cc2oc1=O]

Aspect 19. A compound selected from:

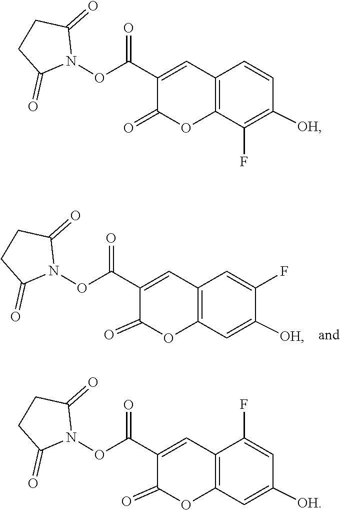 [SMILES: O=C(ON1C(=O)CCC1=O)c1cc2ccc(O)c(F)c2oc1=O ; O=C(ON1C(=O)CCC1=O)c1cc2cc(F)c(O)cc2oc1=O ; O=C(ON1C(=O)CCC1=O)c1cc2c(F)cc(O)cc2oc1=O]

Aspect 20. A method of fluorescently labeling a molecule, wherein the molecule comprises one or more nucleophilic moieties, the method comprising reacting the molecule with a compound of aspect 19.

Aspect 21. The method of aspect 20, wherein the one or more nucleophilic moieties comprise one or more amino groups.

Aspect 22. A method of imaging a sample, the method comprising:
(a) contacting the sample with a compound of any one of aspects 1 to 3;
(b) exposing the sample to light of a wavelength that excites the one or more fluorescent moieties of the compound; and
(c) detecting an optical signal emitted by the compound.

Aspect 23. The method of aspect 22, further comprising:
after (a), (a1) allowing the compound to localize at a target within the sample prior to (b).

Aspect 24. The method of aspect 23, wherein the compound localizes by binding to the target.

Aspect 25. The method of aspect 23 or aspect 24, wherein the optical signal emitted by the compound confirms the presence of the target within the sample.

Aspect 26. The method of any one of aspects 22-25, wherein the sample comprises a biomolecule, a cell, cell culture, a cell lysate, or a tissue sample.

Aspect 27. A method of imaging a subject, the method comprising:
(a) administering to the subject a compound of any one of aspects 1 to 3, wherein upon administration the compound localizes to one or more target sites;
(b) exposing the one or more target sites to light of a wavelength that excites the one or more fluorescent moieties of the compound; and
(c) detecting an optical signal emitted by the compound.

Aspect 28. The method of aspect 27, wherein the subject is a mammal, such as a rat, mouse, or human.

Aspect 29. The method of aspect 27 or aspect 28, wherein the compound localizes to one or more target sites by binding to a target molecule present in the one or more target sites.

Aspect 30. The method of any one of aspects 27-29, wherein the optical signal emitted by the one or more fluorescent moieties confirms the presence of the target molecule within the one or more target sites.

Aspect 31. A method of detecting the presence of a target molecule in a sample, the method comprising:
(a) contacting the sample with a compound of Formula I any one of aspects 4 to 17;
(b) exposing the sample to light of a wavelength that excites Fl of the compound of Formula I; and
(c) detecting an optical signal emitted by the compound of Formula I, wherein the optical signal confirms the presence of the target molecule within the sample.

Aspect 32. The method of aspect 31, wherein the sample comprises a cell, cell culture, cell lysate, or a tissue sample.

Aspect 33. The method of aspect 31 or aspect 32, wherein the compound of Formula I binds to the target molecule.

Aspect 34. A method of detecting the presence of a target molecule in one or more target sites of a subject, the method comprising:
(a) administering to the subject a compound of Formula I of any one of aspects 14 to 17, wherein the compound of Formula I localizes to the one or more target sites comprising the target molecule;
(b) exposing the one or more target sites to light of a wavelength that excites Fl of the compound of Formula I; and
(c) detecting an optical signal emitted by the compound of Formula I, wherein the optical signal confirms the presence of the target molecule in the one or more target sites.

Aspect 35. The method of aspect 34, wherein the subject is a mammal, such as a rat, mouse, or human.

Aspect 36. The method of aspect 34 or aspect 35, wherein the compound of Formula I binds to the target molecule.

Aspect 37. The method of any one of aspects 34-36, wherein the target molecule is associated with the presence, absence, or state of a disease.

Aspect 38. The method of any one of aspects 34-36, wherein the target molecule is associated with a physiological or metabolic state of the subject.

Aspect 39. A method of determining binding affinity between a target molecule and a test compound, the method comprising:
(a) contacting the target molecule with a compound of Formula I of any one of aspects 4 to 17, wherein the compound interacts with the target molecule;
(b) measuring a first optical signal from the compound;
(c) introducing the test compound;
(d) measuring a second optical signal from the compound; and
(e) calculating a difference in the second optical signal and the first optical signal, thereby determining the binding affinity between the target molecule and the test compound.

Aspect 40. The method of aspect 39, wherein the target molecule comprises a target protein.

Aspect 41. The method of aspect 39 or aspect 40, wherein the method is performed in a cell.

Aspect 42. The method of any one of aspects 39-41, wherein the first optical signal, the second optical signal, or both are measured via microscopy or flow cytometry.

Aspect 43. The method of any one of aspects 39-42, wherein the first optical signal, the second optical signal, or both are provided upon exposing the sample to light of a wavelength that excites Fl of the compound.

Aspect 44. A kit comprising a compound of any one of aspects 1-17.

Aspect 45. A compound selected from:

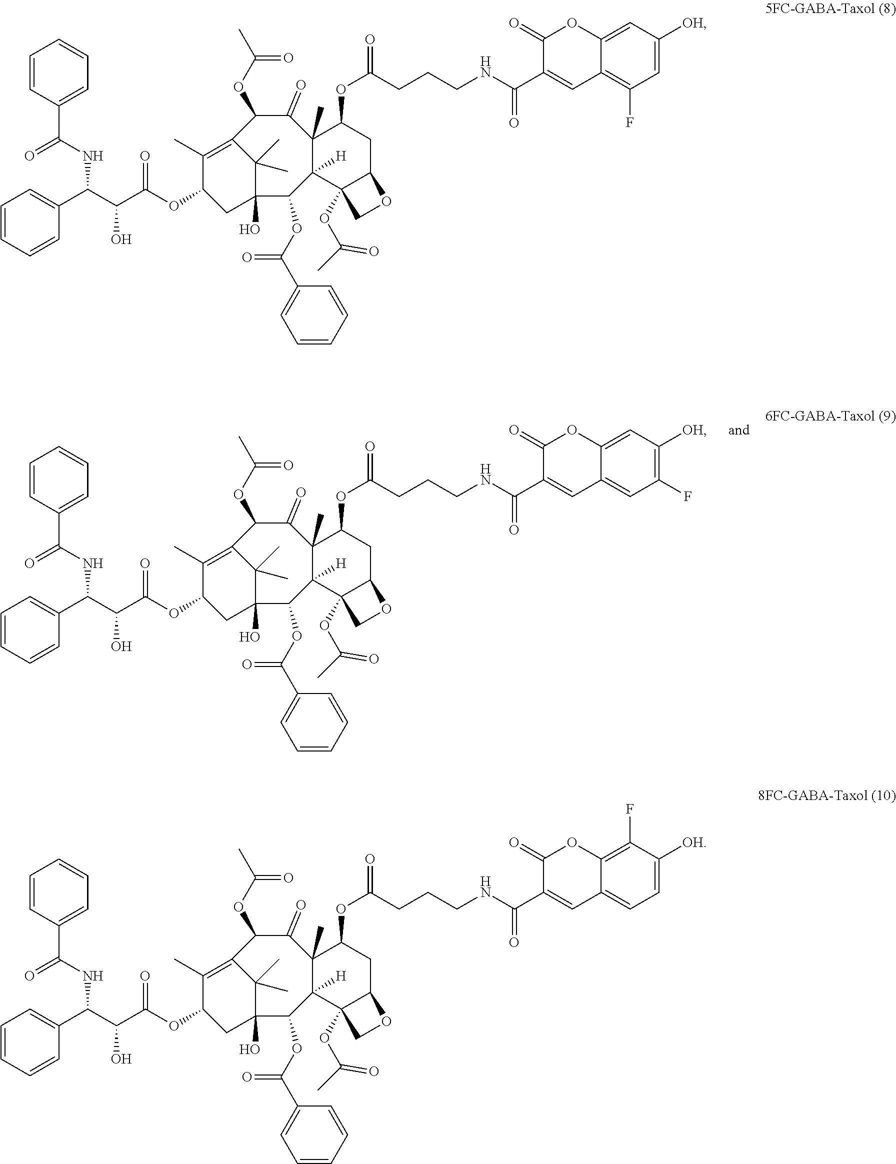

Aspect 46. A compound having the chemical formula

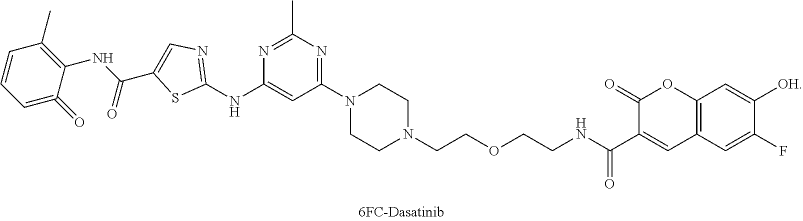

A number of aspects of the disclosure have been described. Nevertheless, it will be understood that various modifications may be made without departing from the spirit and scope of the invention. Accordingly, other aspects are within the scope of the following claims.

By way of non-limiting illustration, examples of certain aspects of the present disclosure are given below.

### Examples

The following examples are set forth below to illustrate the compounds, compositions, and methods claimed herein, along with associated methods and results according to the disclosed subject matter. These examples are not intended to be inclusive of all aspects of the subject matter disclosed herein but rather to illustrate representative methods and results. These examples are not intended to exclude equivalents and variations of the present disclosure, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions, that can be used to optimize the product purity and yield obtained from the described process. Only reasonable and routine experimentation will be required to optimize such process conditions.

### Synthesis of Monofluorinated 7-Hydroxycoumarin-3-Carboxamides as Cell-Permeable Fluorescent Molecular Probes

We synthesized fluorinated derivatives of 7-hydroxycoumarin-3-carboxylic acid (7OHCCA). When linked to the anticancer drug paclitaxel (Taxol) via gamma-aminobutyric acid (GABA), the acidity of these fluorophores profoundly affected cellular efflux and engagement of microtubules in living cells. Compared to PB-GABA-Taxol, bearing the difluorinated Pacific Blue (PB) fluorophore, less acidic coumarins enhanced cellular permeability. This facilitated cellular imaging of fluorescent taxoid probes by confocal microscopy and enabled quantification of probe binding to microtubules without added efflux inhibitors.

### Results and Discussion

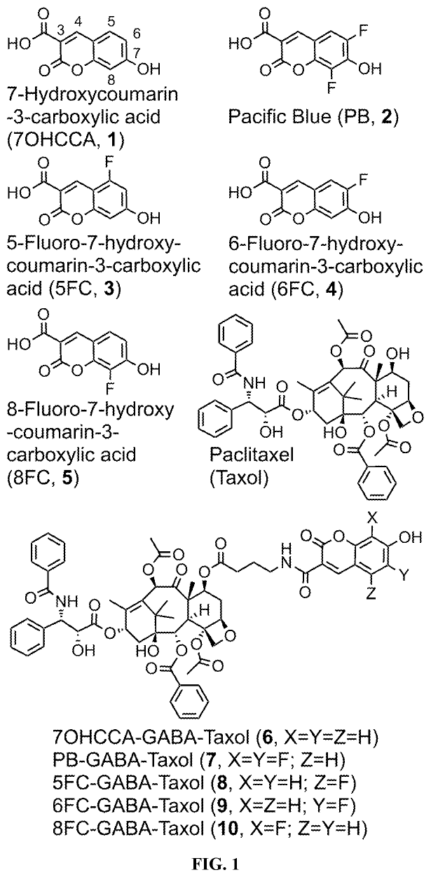

As an approach to improve the photophysical and biological activities of blue-fluorescent molecular probes, we synthesized a series of monofluorinated analogues of 7OHCCA. We modified 7OHCCA with fluorine at the 5-, 6-, and 8-positions to afford 5FC (3), 6FC (4), and 8FC (5, FIG. 1). The synthesis of 6FC (4)¹² and its ethyl ester¹³ from 4-fluororesorcinol has been described, but the fluorescence properties of these compounds were not reported. 8FC (5) is a known compound,⁴ previously termed Jericho Blue, and has been studied as ester and ether derivatives, but amide derivatives of 3-5 have not been characterized. To compare the chemical and photophysical properties of amide derivatives of coumarins 1-5 as spectroscopic standards, we measured pKa values, molar extinction coefficients, and quantum yields of N-hexyl amide derivatives. These studies revealed that in aqueous solution at pH 7.4, amides derived from 6FC (4) are substantially brighter than amides derived from 7OHCCA (1), PB (2), 5FC (3), and 8FC (5). To investigate how substitution with fluorine might affect their biological activities, we additionally linked these coumarins to the anticancer agent paclitaxel (Taxol) via g-aminobutyric acid (GABA). This afforded the novel molecular probes 6 and 8-10 In contrast to the known⁸⁻¹⁰ molecular probe PB-GABA-Taxol (7), these less acidic unsubstituted and monofluorinated coumarin derivatives (6, 8-10) exhibited substantially less active cellular efflux. This facilitated cellular imaging and enabled binding assays of microtubules in living HeLa cells in the absence of efflux inhibitors.

To synthesize coumarin acids 3-5, we modified our previously reported⁷ route used to prepare PB (2) on gram scale. This route to PB (2) beneficially avoids the need to purchase or synthesize costly 2,4-difluororesorcinol by starting with the relatively inexpensive 2,3,4,5-tetrafluorobenzoic acid or the corresponding benzonitrile.^{3, 14} As shown in Scheme 1, monofluorinated benzyl ether protected benzaldehydes (17-19) were prepared by nucleophilic aromatic substitution of three trifluorinated benzonitriles (11-13) with benzyl alcohol, followed by conversion to the benzaldehydes with DIBAL. For our previously reported synthesis of PB (2) using similar methodology,⁷ we hydrogenized 2,4-bis(benzyloxy)-3,5-difluorobenzaldehyde to 3,5-difluoro-2,4-dihydroxybenzaldehyde with palladium on carbon. However, for 17-19, we found that palladium diacetate provided a more selective catalyst for the hydrogenolysis of benzyl groups without any reduction of the aldehyde. Similarly, when palladium diacetate was used for hydrogenolysis of the corresponding precursor to PB (2), 2,4-bis(benzyloxy)-3,5-difluorobenzaldehyde,⁷ this catalyst afforded a 90% yield (0.3 g scale), without any overreduction. This overreduction was occasionally observed when the aldehyde starting material was not sufficiently pure. Cyclization of 20-22 to coumarins 3-5 with Meldrum's acid was followed by synthesis of the amine-reactive NHS esters (23-25). These esters were treated with 1-aminohexane to afford N-hexyl amides (26-28) for subsequent analysis as standards by optical spectroscopy. We previously reported¹⁵ the analogous N-hexyl amides of 7OHCCA (29) and PB (30) for comparison.

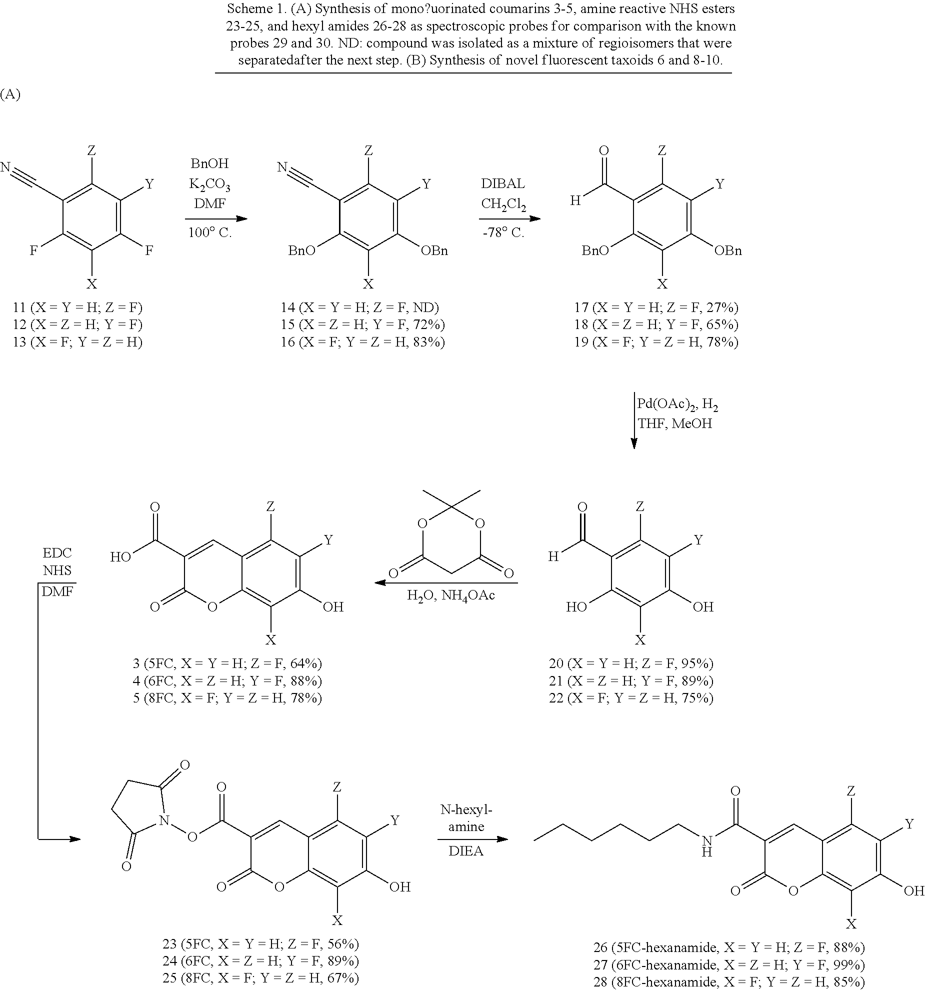

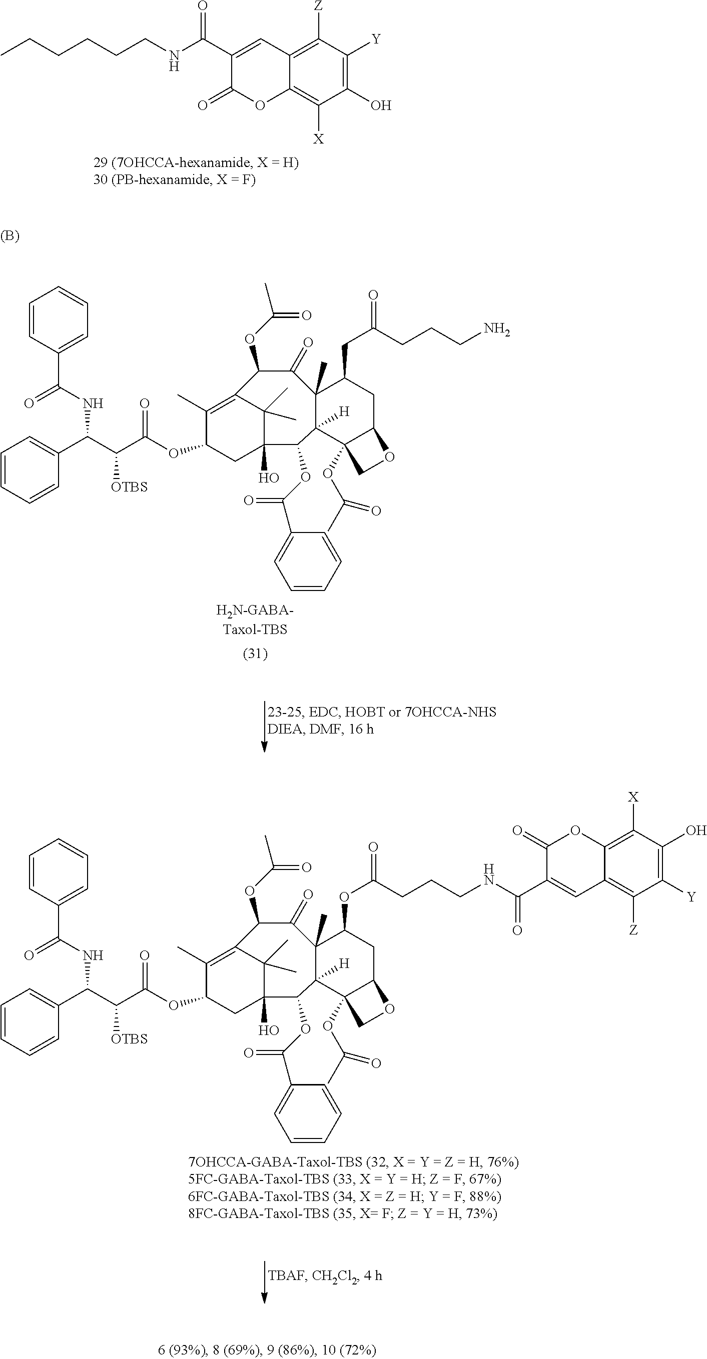

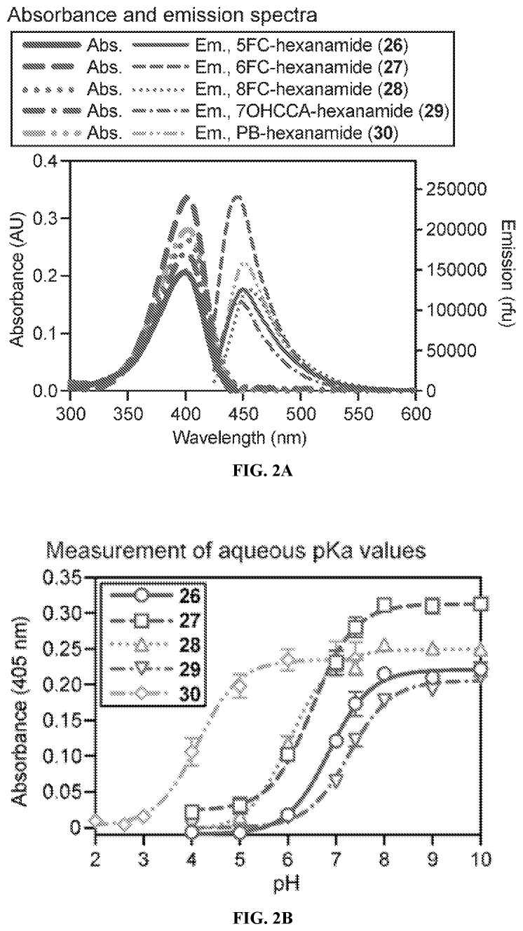

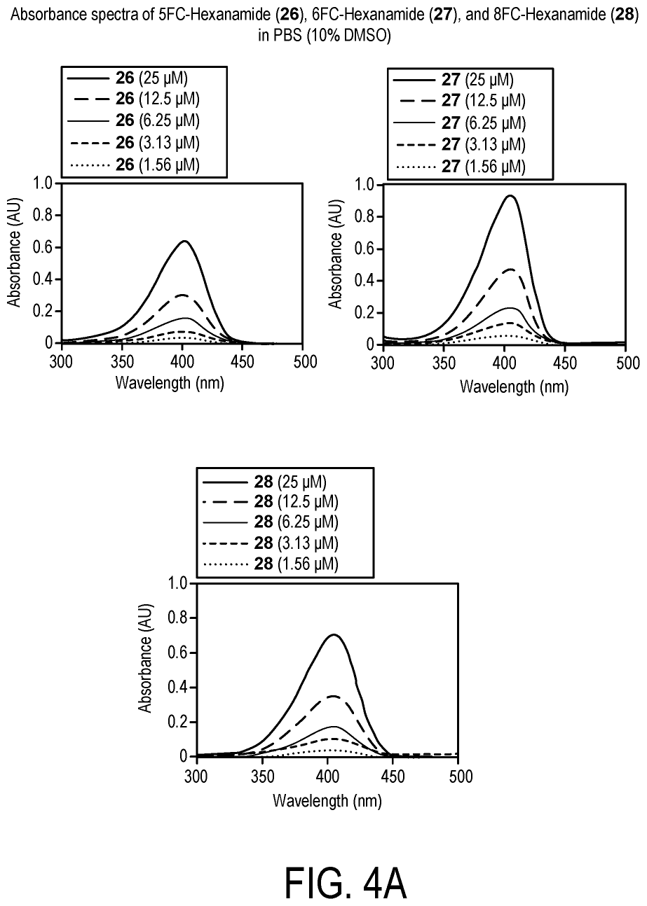

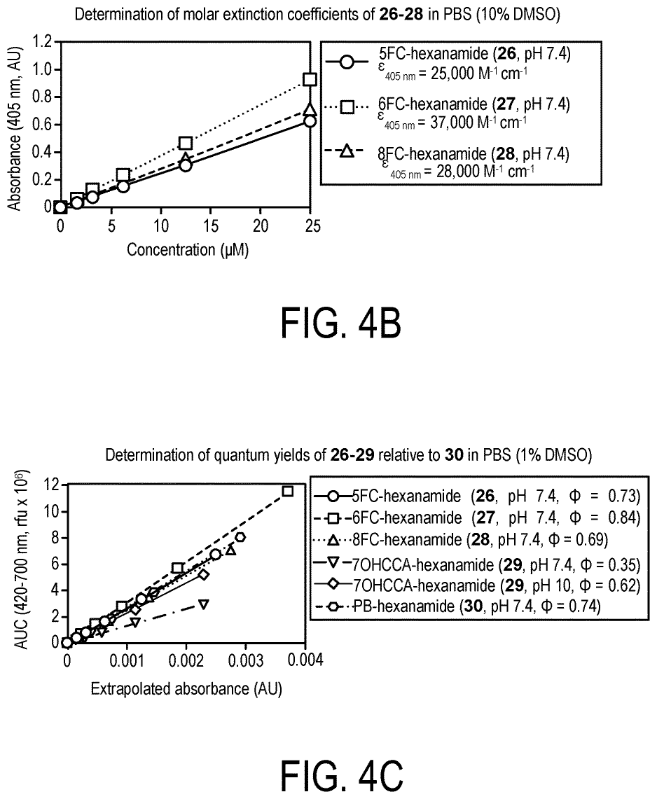

Absorbance and fluorescence emission spectra of probes 26-28 in aqueous buffer compared with 29 and 30 are shown in FIG. 2A. Changes in these spectra as a function of concentration were used to determine the molar extinction coefficients and relative quantum yields of these fluorophores as listed in Table 1 (data shown in FIGS. 4A-4C). These studies revealed a remarkably high molar extinction coefficient of 37,000 M⁻¹ cm⁻¹ and quantum yield of 0.84 for 6FC-hexanamide (27), making it the brightest fluorophore in the series (145% as bright as PB-hexanamide (30). To quantify the acidities of 26-30, the effects of pH on the absorbance spectra of these compounds were analyzed by non-linear regression as shown in FIG. 2B (pKa values are listed in Table 1).

TABLE 1
Photophysical and chemical properties of coumarin amides 26-30 in
aqueous PBS buffer. Molar extinction coefficients were measured
at 405 nm in PBS containing 10% DMSO to facilitate determination
of concentrations of DMSO stock solutions by 10-fold dilution into
PBS. Other values were measured in PBS (1% DMSO). Molar extinction
coefficients for 29 and 30 were previously described.¹⁵

| | Abs./Em. | ε_{405 nm} | | | |
|---|---|---|---|---|---|
| | λₘₐₓ | (M⁻¹ cm⁻¹, | | | |
| Compound | (nm, pH 7.4) | 10% DMSO, pH) | Φ (pH) | | pKa |
| 5FC-hexanamide | 400/449 | 25,000 (7.4) | 0.73 | (7.4) | 6.9 |
| (26) | | | | | |
| 6FC-hexanamide | 401/451 | 37,000 (7.4) | 0.84 | (7.4) | 6.5 |
| (27) | | | | | |
| 8FC-hexanamide | 402/457 | 28,000 (7.4) | 0.69 | (7.4) | 6.0 |
| (28) | | | | | |
| 7OHCCA- | 401/447 | 22,000 (10) | 0.35 | (7.4) | 7.3 |
| hexanamide (29) | | | 0.62 | (10) | |
| PB- | 403/447 | 29,000 (7.4) | 0.74^{‡} | (7.4) | 4.1 |
| hexanamide (30) | | | | | |

^{‡}The QY shown for 30 is based on a previously reported^{4, 7} PB-biotin amide. pKa and other values were measured with at least two replicates, and confidence intervals (95%) for pKa values are ±0.1 for 26-29 and ±0.2 for 30.

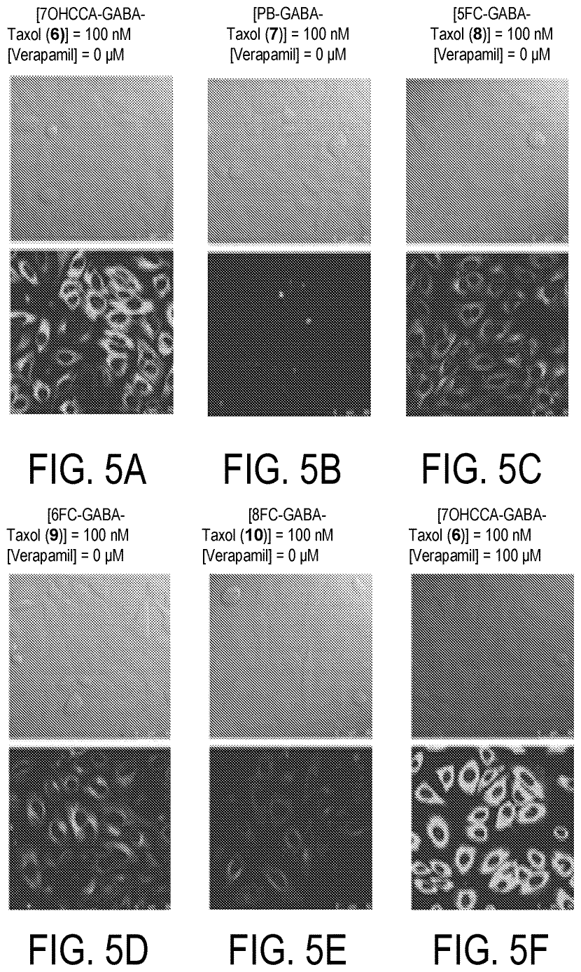

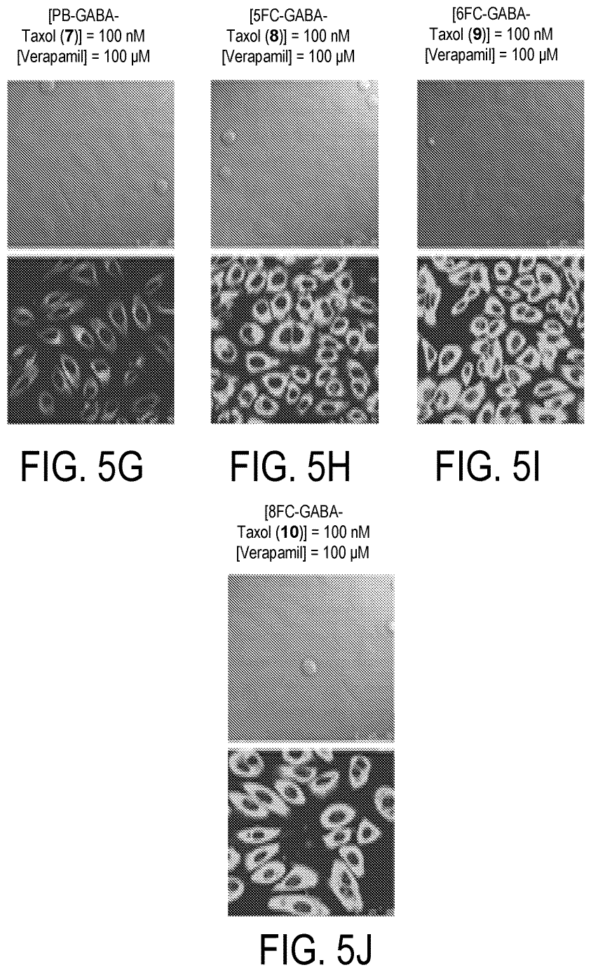

To investigate how monofluorinated coumarins might affect the biological properties of molecular probes, we linked these fluorophores and 7OHCCA to paclitaxel (Taxol) by amidation of H2N-GABA-Taxol-TBS⁸ (31, Scheme 1) followed by removal of the TBS group. Paclitaxel was chosen for these studies because fluorescent analogues are of significant interest as probes of mechanisms of action of this drug and related tubulin-binding anticancer agents.¹⁶⁻²² Examination of HeLa cells treated with the fluorescent taxoids 6-10 by confocal microscopy revealed substantial differences in cellular uptake in the absence and presence of verapamil (images shown in FIGS. 5A-5J). When efflux was inhibited by co-treatment with verapamil (100 μM), all of these probes showed strong association with intracellular microtubules. However, PB-GABA-Taxol (7) exhibited the lowest cellular fluorescence, due to its high sensitivity to residual active efflux, as previously reported.⁸ In contrast, in the absence of verapamil, PB-GABA-Taxol (7) showed very little cellular uptake due to active efflux, 7OHCCA-GABA-Taxol (6) exhibited high cellular uptake, and the monofluorinated coumarins (8-10) showed intermediate levels of uptake as evidenced by their labeling of cellular microtubules. These patterns of cellular permeability paralleled the predicted hydrophobicities (c Log D values) of these probes as calculated with ChemAxon Marvin software (v. 23.3.0). For this analysis, the ChemAxon method was used with ChemAxon-calculated pKa values that were within one pKa unit of the experimental values measured for 26-30 (Table 1). Correspondingly, the most acidic probe (7) was calculated to exhibit the highest predicted polarity (c Log D_{pH7.4}=2.2; calculated pKa (ChemAxon)=5.1) and the least acidic probe (6) the lowest predicted polarity (c Log D_{pH7.4}=6.7; calculated pKa (ChemAxon)=7.7). Intermediate polarities were calculated for monofluorinated coumarins 8 (c Log_{DpH7.4}=3.0; calculated pKa (ChemAxon)=6.2), 9 (c Log_{DpH7.4}=3.0; calculated pKa (ChemAxon)=6.2, and 10 (c Log D_{pH7.4}=3.1; calculated pKa (ChemAxon)=6.4). For comparison, the drug paclitaxel was calculated with the same method to exhibit c Log P=3.3. These results indicate that the monofluorinated coumarin probes most closely mimic the polarity of paclitaxel.

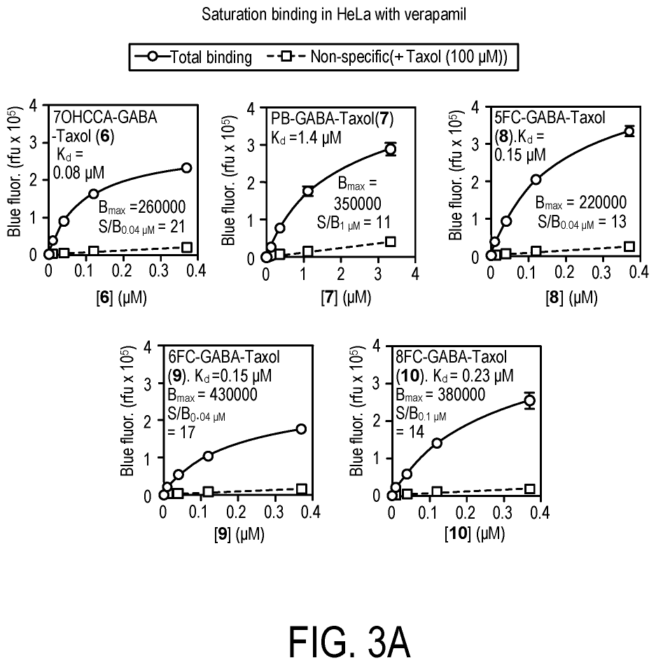

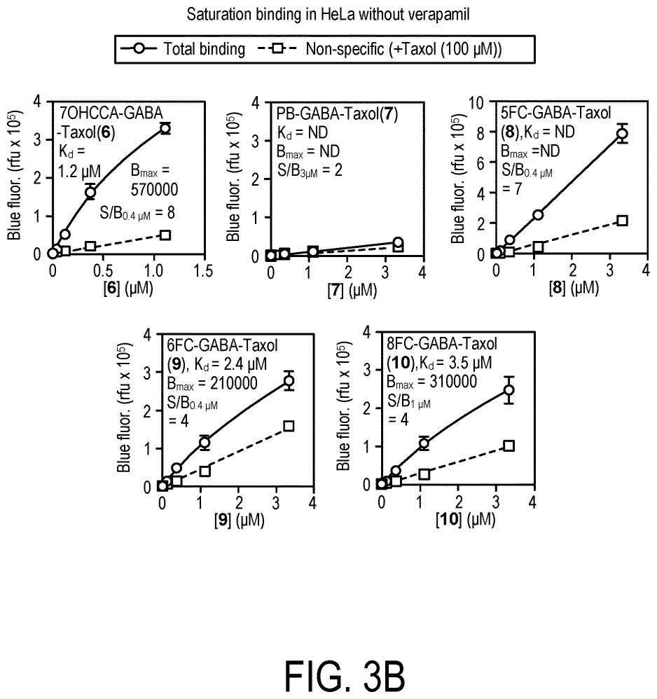

In biochemical assays, PB-GABA-Taxol (7) binds cross-linked microtubules with K_{d}=265 nM.⁸ In living HeLa cells, this probe can also be used to quantify interactions of small molecules with microtubules by flow cytometry in the presence of verapamil.⁹ To evaluate the cellular affinities of the less acidic fluorescent taxoids (6, 8-10) for microtubules, we treated living HeLa cells with these compounds and 7 as a control and used previously described⁹ saturation binding assays to quantify binding to microtubules by flow cytometry. In these assays, total binding was measured by varying the probe concentration and non-specific binding was measured by the addition of excess paclitaxel (100 μM) to block specific binding of the fluorescent probe. For curve fitting, the lowest maximal concentration that achieved ca. 50% saturation was used to minimize the effect of these probes on living cells. This approach provided the most consistent measurements of both cellular affinities and Bₘₐₓ values, a measure of binding sites in cells. Bₘₐₓ is additionally affected by the brightness of the specific fluorophore of these probes in the intracellular environment. Cellular dissociation constants (K_{d}) were measured both in the presence and absence of verapamil using non-linear regression with a one-site total and non-specific binding model (GraphPad Prism) as shown in FIGS. 3A and 3B.

Consistent with confocal microscopy (FIGS. 5A-5J), the efflux inhibitor verapamil (100 μM) enhanced the apparent affinities of all fluorescent taxoids for cellular microtubules. As shown in FIG. 3A, in the presence of verapamil the monofluorinated coumarin probes (8-10) exhibited similar affinities, with K_{d} values within 2-fold of each other (cellular K_{d} (8)=0.15 μM; cellular K_{d} (9)=0.15 μM; cellular K_{d} (10)=0.23 μM). These affinities were substantially higher than the more polar PB-GABA-Taxol (cellular K_{d} (7)=1.4 μM). Correspondingly, the most hydrophobic taxoid (6) exhibited the highest affinity for microtubules in cells (cellular K_{d} (6)=0.08 μM) when efflux was inhibited. This value is within ca. 5-fold or less of the affinity of paclitaxel itself for purified microtubules (biochemical K_{d}=15 nM,²³ Kᵢ=19 nM,²⁴ Kᵢ=27 nM,²⁵ Kᵢ=31 nM,²⁵ K_{d}=50 nM,² and K_{d}=70 nM²⁷).

In the absence of verapamil, active efflux prevented quantification of the affinity of PB-GABA-Taxol (7) for microtubules (FIG. 3B). However, the greater cellular permeability and intracellular accumulation of three of the less acidic taxoids enabled quantifications of these interactions. The least active of these compounds was probe 8, which exhibited a substantially higher signal-to-background (S/B_{0.4 μM}=7-fold) than 7 (S/B_{3 μM}=2-fold), but its low affinity prevented accurate determination of cellular K_{d}. In contrast, probes 6, 9, and 10 bound more tightly to microtubules, allowing quantification of affinity in cells (cellular K_{d} (6)=1.2 μM; cellular K_{d} (9)=2.4 μM; cellular K_{d} (10)=3.5 μM). Additionally, the 7OHCCA-derived probe 6 exhibited the greatest signal-to-background (S/B_{0.4 μM}=8-fold).

In summary, we synthesized and investigated a series of monofluorinated coumarins (3-5) and related fluorescent taxoids as molecular probes (6-10) of cellular microtubules. We found that the 6FC (4) fluorophore, derivatized as a hexyl amide (27), exhibited a remarkably high molar extinction coefficient of 37,000 M⁻¹ cm⁻¹ and quantum yield of 0.84. The brightness of this fluorophore, calculated as the product of the molar extinction coefficient and quantum yield, was 145% greater than a hexanamide derivative of Pacific Blue (2), one of the brightest commercially available coumarins. Measurements of the pKa values of these monofluorinated fluorophores indicate that they will be predominantly anionic under physiological conditions, which can beneficially reduce non-specific binding to biomolecules. To investigate effects on biological activity, we synthesized four novel fluorescent analogues of paclitaxel. Properties calculations predicted that taxoids linked to monofluorinated coumarin amides can exhibit hydrophobicities (c Log D (8-10)_{pH 7.4}=3.0-3.1) that are highly similar to the parent drug paclitaxel (c Log P (paclitaxel)=3.3). Studies of the differential sensitivities of fluorescent taxoids 6-10 to active cellular efflux revealed that monofluorinated coumarins are substantially less sensitive to efflux compared to the difluorinated fluorophore Pacific Blue, enabling higher cellular permeability and engagement of cellular microtubules. Taxoid 6, derived from the non-fluorinated 7OHCCA fluorophore, exhibited the lowest sensitivity to active efflux and the greatest affinity for microtubules in living cells both in the presence (cellular K_{d} (6)=0.08 μM) and absence (cellular K_{d} (6)=1.2 μM) of the efflux inhibitor verapamil. However, this probe (6) is predicted to be over 1000-fold more hydrophobic (c Log D (6)_{pH 7.4}=6.7) than paclitaxel.

This high hydrophobicity may enhance non-specific interactions with other biomolecules and might affect the ability of this probe to faithfully mimic some biological properties of paclitaxel. The exceptionally high fluorescence brightness of derivatives of 6FC (4), and its decreased susceptibility to efflux compared to PB, makes this monofluorinated fluorophore particularly attractive for the synthesis of cell-permeable molecular probes that engage intracellular target proteins.

### Biological Assays and Protocols

Cell culture: HeLa cells (CCL-2) were purchased from ATCC, and cultured in Dulbecco's Modified Eagle Medium (DMEM, Sigma D6429). Media was supplemented with fetal bovine serum (FBS, 10%, Fisher Scientific, NC0924828), penicillin (100 units/mL), and streptomycin (100 μg/mL, Sigma P4333). Cells were maintained in a humidified 5% CO₂ incubator at 37° C.

Confocal microscopy: Cells were added to an 8-well chambered coverslip (Ibidi IbiTreat μ-Slide, 300 μL, 20,000 cells/well) and allowed to proliferate for 24 h prior to addition of compounds. Compounds in DMSO stock solutions were serially diluted 1,000-fold in complete medium (to 0.1% DMSO) prior to addition to cells. Cells were treated with compounds at 37° C. for 3 h before imaging without washing with a Leica SP8 confocal laser-scanning microscope (63× oil-immersion objective).

Flow cytometry: Cells were analyzed with a Beckman Coulter Cytoflex S (B2-R0-V2-Y2) flow cytometer. Fluorophores were excited with a 405 nm laser and emitted photons were collected through 450/45 nm BP, filter (FSC threshold=500,000, flow speed=fast, mixing time=3 s, backflush time=6 s, gain=5, and 10,000 cells were analyzed). Affinities of probes 6-10 for microtubules in living HeLa cells were measured after treatment for 3 h without washing as previously described in Andres, A. E., et al. ACS Bio & Med Chem Au 2022, 2 (5), 529-537.

General experimental section: Chemicals were purchased from Sigma Aldrich, Acros Organics, Alfa Aesar, Oakwood Chemical, Ambeed, Inc. or Chem Impex International. All nonaqueous reactions were carried out using flame- or oven-dried glassware under an atmosphere of dry argon or nitrogen. Tetrahydrofuran (THF), dichloromethane (CH₂Cl₂), N,N-dimethylformamide (DMF), and methanol (CH₃OH) were purified via filtration through two columns of activated basic alumina under an atmosphere of Argon using a solvent purification system from Pure Process Technology. Other commercial reagents were used as received unless otherwise noted. ¹H Nuclear magnetic resonance (¹H NMR), ¹³C NMR, and ¹⁹F NMR spectra were acquired on a Bruker Avance Neo (400 MHz), Avance III HD (400 MHz) or Bruker Avance III Ascend (700 MHz) instruments. For ¹H and ¹³C, chemical shifts (δ) are reported in ppm referenced to CDCl₃ (7.26 ppm for ¹H and 77.2 ppm for ¹³C), CD₃OD (3.31 ppm for ¹H, 49.0 ppm for ¹³C), or dimethyl sulfoxide (DMSO)-d₆ (2.50 ppm for ¹H, 39.5 ppm for ¹³C). For ¹⁹F, chemical shifts (6) are reported in ppm referenced to trifluoroethanol (−77.0 ppm for 19F). ¹H coupling constants (JHH, Hz), ¹³C coupling constants (JCF, Hz), and ¹⁹F coupling constants (JFF, Hz) are reported as chemical shift, multiplicity (br=broad, s=singlet, d=doublet, t=triplet, q=quartet, and m=multiplet), coupling constant, and integration. High-resolution mass spectra (HRMS) were obtained at the Mass Spectrometry Laboratory at the The Ohio State University, College of Pharmacy on a Thermo Q-Exactive Orbitrap with Vanquish-H UHPLC. Thin layer chromatography (TLC) was performed using EMD aluminum-backed (0.20 mm) silica plates (60 F-254), and flash chromatography used ICN silica gel (200-400 mesh). TLC plates were visualized with a UV lamp or by staining with 12. PB-GABA-Taxol was prepared as previously described in Lee, M. M. et al. Angew. Chem. Int. Ed. Engl. 2017, 56 (24), 6927-6931 and Andres, A. E., et al. Methods. Mol. Biol. 2022, 2430, 449-466. Pacific Blue-NHS was prepared as previously described in Lee, M. M. et al. ACS Omega 2016, 1 (6), 1266-1276, except Pd(OAc)₂ was used as a more selective catalyst for the synthesis of 3,5-difluoro-2,4-dihydroxybenzaldehyde from 2,4-bis(benzyloxy)-3,5-difluorobenzaldehyde. pKa values were measured by absorbance spectroscopy (405 nm, 10 μM, 1% DMSO) in aqueous citric acid-sodium phosphate (pH 2.0-7.4), potassium phosphate (pH 7.6-8.0), and sodium carbonate-sodium bicarbonate (pH 9.2-10.8) buffers.

### Synthetic Procedures and Compound Characterization Data

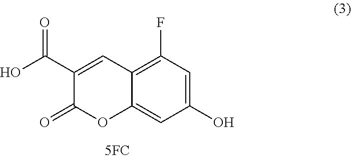 [SMILES: O=C(O)c1cc2c(F)cc(O)cc2oc1=O]

5-Fluoro-7-hydroxy-2-oxo-2H-chromene-3-carboxylic acid (5FC, 3). To a solution of 2-fluoro-4,6-dihydroxybenzaldehyde (20, 0.2 g, 1.28 mmol) in water (5 mL), was added Meldrum's acid (203 mg, 1.41 mmol) and ammonium acetate (34.6 mg, 448 μmol). The reaction mixture was stirred at 22° C. for 3-4 h in the dark and the progress was monitored by TLC. Subsequently, aqueous HCl (2 M, 15 mL) was added to the reaction mixture and the reaction cooled to 4° C. and stirred for 1 h. A precipitate that formed was filtered, washed with cold water (2×10 mL) and dried under high vacuum to afford 5FC (3) as a pale brown solid. Yield: 185 mg (64%); ¹H NMR (400 MHz, DMSO) δ 13.05 (s, 1H), 11.51 (s, 1H), 8.50 (d, J=0.7 Hz, 1H), 6.70 (dd, J=11.3, 2.1 Hz, 1H), 6.61 (dd, J=2.1, 1.1 Hz, 1H); ³C NMR (101 MHz, DMSO) δ 164.6 (d, J=14.5 Hz), 163.8, 159.9 (d, J=254.7 Hz), 156.7 (d, J=8.0 Hz), 156.4, 141.4 (d, J=3.5 Hz), 113.1, 100.7 (d, J=19.6 Hz), 100.0 (d, J=21.8 Hz), 98.7 (d, J=3.3 Hz); ¹⁹F NMR (377 MHz, DMSO) δ −117.1; HRMS (ESI): caled for (C₁₀H₅FO₅+H⁺), 225.01938; found 225.01911 [M+H]⁺.

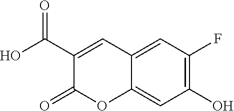 [SMILES: O=C(O)c1cc2cc(F)c(O)cc2oc1=O]

6-Fluoro-7-hydroxy-2-oxo-2H-chromene-3-carboxylic acid (6FC, 4). To a solution of 5-fluoro-2,4-dihydroxybenzaldehyde (21, 130 mg, 833 mol) in water (5 mL), was added Meldrum's acid (120 mg, 833 μmol) and ammonium acetate (22.5 mg, 833 mol). The reaction mixture was stirred at 22° C. for 3-4 h in the dark and the progress was monitored by TLC. Subsequently, aqueous HCl (2 M, 15 mL) was added and the reaction cooled to 4° C. and stirred for 1 h. A precipitate that formed was filtered, washed with cold water (2×10 mL) and dried under high vacuum to afford 6FC (4) as a pale yellow solid; Yield: 165 mg (88%); ¹H NMR (400 MHz, DMSO) δ 12.98 (s, 1H), 11.62 (s, 1H), 8.65 (s, 1H), 7.77 (d, J=10.8 Hz, 1H), 6.92 (d, J=7.2 Hz, 1H); ¹³C NMR (101 MHz, DMSO) δ 164.0, 157.1, 152.8, 151.8 (d, J=14.6 Hz), 148.7 (d, J=2.8 Hz), 148.3 (d, J=241.2 Hz), 115.7 (d, J=20.7 Hz), 114.0, 109.8 (d, J=8.7 Hz), 104.1 (d, J=2.6 Hz); ¹⁹F NMR (377 MHz, DMSO) δ −139.5; HRMS (ESI): calcd for (C₁₀H₅FO₅+H⁺), 225.01938; found 225.01902 [M+H]⁺.

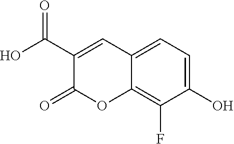 [SMILES: O=C(O)c1cc2ccc(O)c(F)c2oc1=O]

8-Fluoro-7-hydroxy-2-oxo-2H-chromene-3-carboxylic acid (8FC, 5). To a solution of 3-fluoro-4,6-dihydroxybenzaldehyde (22, 0.2 g, 1.28 mmol) in water (5 mL), was added Meldrum's acid (203 mg, 1.41 mmol) and ammonium acetate (34.6 mg, 448 μmol). The reaction mixture was stirred at 22° C. for 3-4 h in the dark and the progress was monitored by TLC. Subsequently, aqueous HCl (2 M, 15 mL) was added and the reaction cooled to 4° C. and stirred for 1 h. A precipitate that formed was filtered, washed with cold water (2×10 mL) and dried under high vacuum to afford 8FC (5) as a pale brown solid. Yield: 225 mg (78%); ¹H NMR (400 MHz, DMSO) δ 13.03 (s, 1H), 11.48 (s, 1H), 8.70 (s, 1H), 7.56 (dd, J=8.8, 1.7 Hz, 1H), 6.99 (dd, J=8.7, 7.5 Hz, 1H); ¹³C NMR (101 MHz, DMSO) δ 164.0, 156.0, 150.9 (d, J=8.8 Hz), 149.2 (d, J=2.5 Hz), 144.3 (d, J=8.4 Hz), 137.4 (d, J=244.5 Hz), 126.0 (d, J=3.7 Hz), 114.3 (d, J=1.9 Hz), 113.6, 111.3; ¹⁹F NMR (377 MHz, DMSO) δ −160.0; HRMS (ESI): calcd for (C₁₀H₅FO₅+H⁺), 225.01938; found 225.01910 [M+H]⁺.

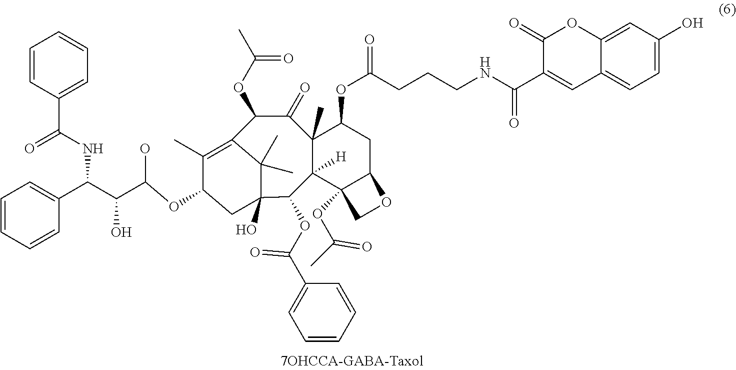

(2aR,4S,4aS,6R,9S,11S,12S,12bS)-9-(((2R,3S)-3-benzamido-2-hydroxy-3-phenylpropanoyl)oxy)-12-(benzoyloxy)-11-hydroxy-4-((4-(7-hydroxy-2-oxo-2H-chromene-3-carboxamido)butanoyl)oxy)-4a,8,13,13-tetramethyl-5-oxo-3,4,4a,5,6,9,10,11,12,12a-decahydro-1H-7,11-methanocyclodeca[3,4]benzo[1,2-b]oxete-6,12b(2aH)-diyldiacetate (7OHCCA-GABA-Taxol, 6). To 7OHCCA-GABA-Taxol-TBS (32, 33 mg, 26.6 μmol) in CH₂Cl₂ (2.0 mL) was added tetrabutylammonium fluoride (1 M in THF, 80 μL, 80 mol). The reaction mixture was stirred at 22° C. for 16 h. The solvent was removed under reduced pressure, and the residue was dissolved in MeOH (1 mL The product was purified by reverse phase chromatography on a Teledyne ISCO Combiflash instrument (C18 column, H₂O and CH₃CN, 0.1% formic acid v/v, gradient: H₂O:CH₃CN (90:10) to (0:100) over 20 min, elution time 7-8 min). Pure fractions were collected and combined, and the solvent was removed using lyophilization for 16 h to afford 7OHCCA-GABA-Taxol (6) as a colorless solid; Yield: 28 mg (93%); ¹H NMR (400 MHz, CDCl₃) δ 8.93 (t, J=5.8 Hz, 1H), 8.74 (s, 1H), 8.11-8.04 (m, 2H), 7.80-7.73 (m, 2H), 7.63-7.54 (m, 1H), 7.53-7.43 (m, 7H), 7.42-7.35 (m, 4H), 7.35-7.28 (m, 2H), 6.91-6.85 (m, 1H), 6.84-6.81 (m, 1H), 6.21 (s, 1H), 6.16 (t, J=8.8 Hz, 1H), 5.83-5.75 (m, 1H), 5.65 (d, J=6.8 Hz, 1H), 5.61-5.52 (m, 1H), 4.93-4.86 (m, 1H), 4.82 (d, J=2.8 Hz, 1H), 4.28 (d, J=8.5 Hz, 1H), 4.16 (d, J=8.5 Hz, 1H), 3.90 (d, J=6.7 Hz, 1H), 3.45 (q, J=6.9 Hz, 2H), 2.62-2.40 (m, 2H), 2.35 (s, 3H), 2.35-2.23 (m, 2H), 2.16 (s, 3H), 2.01-1.69 (m, 9H), 1.19 (s, 3H), 1.15 (s, 3H); 13C NMR (101 MHz, CDCl₃) δ 202.1, 172.5 (2 carbons), 170.5, 169.3, 167.6, 167.0, 163.4, 163.0, 162.0, 156.8, 148.8, 140.5, 138.2, 133.9, 133.7, 133.1, 132.2, 131.7, 130.3 (2 carbons), 129.2, 129.1 (2 carbons), 128.9 (2 carbons), 128.8, 128.4, 127.2 (3 carbons), 115.0, 113.8, 112.0, 102.9, 84.0, 81.2, 78.6, 77.4, 76.6, 75.4, 74.5, 73.4, 72.1, 71.6, 56.3, 55.3, 47.2, 43.4, 40.9, 39.3, 35.7, 33.6, 31.4, 26.7, 24.3, 22.7, 21.0, 20.9, 14.8, 11.0; HRMS (ESI): calcd for (C₆₁H₆₂N₂O₁₉−H⁺), 1125.38740; found 1125.38913 [M−H]⁻.

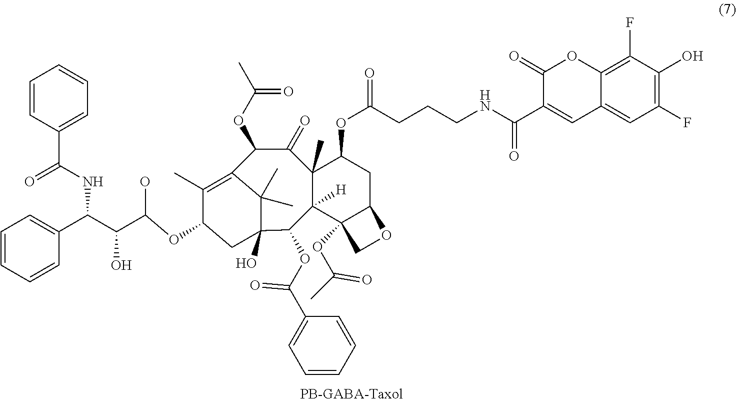

PB-GABA-Taxol (7). This known compound was prepared as previously described in Angew. Chem. Int. Ed. Engl. 2017, 56 (24), 6927-6931.

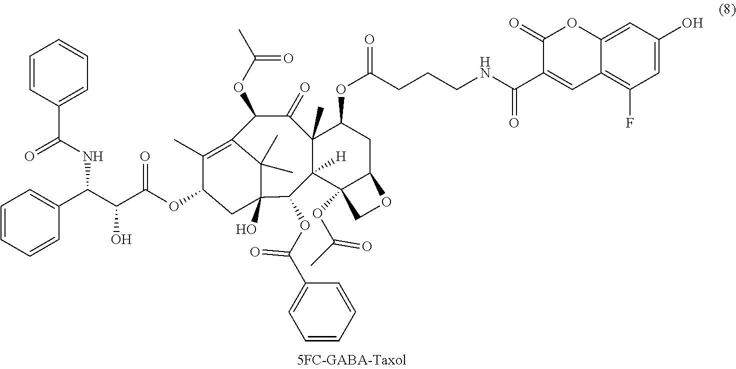

(2aR,4S,4aS,6R,9S,11S,12S,12bS)-9-(((2R,3S)-3-benzamido-2-hydroxy-3-phenylpropanoyl)oxy)-12-(benzoyloxy)-4-((4-(5-fluoro-7-hydroxy-2-oxo-2H-chromene-3-carboxamido)butanoyl)oxy)-11-hydroxy-4a,8,13,13-tetramethyl-5-oxo-3,4,4a,5,6,9,10,11,12,12a-decahydro-1H-7,11-methanocyclodeca[3,4]benzo[1,2-b]oxete-6,12b(2aH)-diyl diacetate (5FC-GABA-Taxol, 8). To 5FC-GABA-Taxol-TBS (33, 16 mg, 12.7 μmol) in CH₂Cl₂ (2.0 mL) was added tetrabutylammonium fluoride (1 M in THF, 38 μL, 38 μmol). The reaction mixture was stirred at 22° C. for 4 h. The solvent was removed under reduced pressure and the residue was dissolved in MeOH (1 mL). The product was purified by reverse phase chromatography on a Teledyne ISCO Combiflash instrument (C18 column, H₂O and CH₃CN, 0.1% formic acid v/v, gradient: H₂O:CH₃CN (90:10) to (0:100) over 20 min, elution time 7-8 min). Pure fractions were collected and combined, and the solvent was removed using lyophilization for 16 h to afford 5FC-GABA-Taxol (8) as a colorless solid; Yield: 10 mg (69%). ¹H NMR (700 MHz, DMSO) δ 8.93 (d, J=8.6 Hz, 1H), 8.59 (t, J=5.9 Hz, 1H), 8.54 (s, 1H), 8.00-7.95 (m, 2H), 7.91-7.86 (m, 2H), 7.75-7.70 (m, 1H), 7.66-7.61 (m, 2H), 7.57-7.53 (m, 1H), 7.52-7.46 (m, 2H), 7.44-7.36 (m, 4H), 7.26-7.18 (m, 1H), 6.60-6.50 (m, 1H), 6.46 (s, 1H), 6.20 (s, 1H), 6.07 (s, 1H), 5.93-5.86 (m, 1H), 5.48-5.38 (m, 3H), 5.00-4.92 (m, 1H), 4.80 (s, 1H), 4.60 (d, J=7.8 Hz, 1H), 4.09-4.00 (m, 2H), 3.71 (d, J=7.1 Hz, 1H), 3.28-3.22 (m, 2H), 2.47-2.39 (m, 1H), 2.28-2.18 (m, 5H), 2.10 (s, 3H), 1.92-1.85 (m, 1H), 1.80-1.60 (m, 10H), 1.04 (s, 3H), 0.98 (s, 3H); ¹³C NMR (176 MHz, DMSO) δ 201.7, 172.7, 171.5, 170.1, 168.8, 166.2, 165.2, 161.6, 160.8, 160.2 (d, J=254.2 Hz), 156.5 (d, J=8.4 Hz), 140.0, 139.8, 139.2, 134.5, 133.5, 132.5, 131.3, 129.8, 129.6, 128.7, 128.3 (2 carbons), 128.3 (3 carbons), 127.5 (3 carbons), 127.4 (4 carbons), 101.2 (d, J=18.7 Hz), 100.2, 99.2, 82.8, 79.7, 76.7, 75.2, 74.6, 74.1, 73.6, 71.0, 69.4, 56.4, 55.3, 46.1, 43.0, 40.0, 38.1, 34.6, 32.8, 30.9, 26.2, 24.1, 22.4, 21.2, 20.4, 13.8, 10.6; ¹⁹F NMR (377 MHz, CDCl₃) δ −114.7; HRMS (ESI): calcd for (C₆₁H₆₁FN₂O₁₉−H⁺), 1143.37798; found 1143.38079 [M−H]⁺.

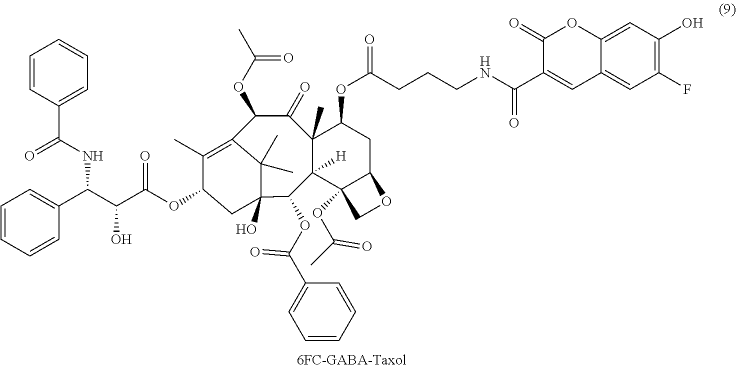

(2aR,4S,4aS,6R,9S,11S,12S,12bS)-9-(((2R,3S)-3-benzamido-2-hydroxy-3-phenylpropanoyl)oxy)-12-(benzoyloxy)-4-((4-(6-fluoro-7-hydroxy-2-oxo-2H-chromene-3-carboxamido)butanoyl)oxy)-11-hydroxy-4a,8,13,13-tetramethyl-5-oxo-3,4,4a,5,6,9,10,11,12,12a-decahydro-1H-7,11-methanocyclodeca[3,4]benzo[1,2-b]oxete-6,12b(2aH)-diyl diacetate (6FC-GABA-Taxol, 9). To 6FC-GABA-Taxol-TBS (34, 36 mg, 28.6 μmol) in CH₂Cl₂ (2.0 mL) was added tetrabutylammonium fluoride (1 M in THF, 86 μL, 86 gmol). The reaction mixture was stirred at 22° C. for 4 h. The solvent was removed under reduced pressure and the residue dissolved in MeOH (1 mL). The product was purified by reverse phase chromatography on a Teledyne ISCO Combiflash instrument (C18 column, H₂O and CH₃CN, 0.1% formic acid v/v, gradient: H₂O:CH₃CN (90:10) to (0:100) over 20 min, elution time 7-8 min). Pure fractions were collected and combined, and the solvent was removed using lyophilization for 16 h to afford 6FC-GABA-Taxol (9) as a colorless solid; Yield: 28 mg (86%). ¹H NMR (700 MHz, DMSO) δ 8.93 (d, J=8.6 Hz, 1H), 8.73 (s, 1H), 8.65 (t, J=6.0 Hz, 1H), 8.00-7.94 (m, 2H), 7.91-7.86 (m, 2H), 7.80 (d, J=10.8 Hz, 1H), 7.75-7.70 (m, 1H), 7.67-7.61 (m, 2H), 7.57-7.53 (m, 1H), 7.52-7.46 (m, 2H), 7.42-7.37 (m, 4H), 7.26-7.20 (m, 1H), 6.91 (d, J=7.1 Hz, 1H), 6.19 (d, J=7.5 Hz, 1H), 6.07 (s, 1H), 5.93-5.85 (m, 1H), 5.48-5.37 (m, 3H), 4.99-4.91 (m, 1H), 4.80 (s, 1H), 4.64-4.56 (m, 1H), 4.11-3.96 (m, 2H), 3.71 (d, J=7.1 Hz, 1H), 3.31-3.23 (m, 2H), 2.47-2.38 (m, 1H), 2.29-2.17 (m, 5H), 2.09 (s, 3H), 1.93-1.84 (m, 1H), 1.81-1.63 (m, 10H), 1.04 (s, 3H), 0.98 (s, 3H); ¹³C NMR (176 MHz, DMSO) δ 201.7, 172.7, 171.5, 170.1, 168.8, 166.2, 165.2, 161.5, 160.9, 152.4, 148.9 (d, J=241.2 Hz), 147.3 (d, J=2.9 Hz), 140.0, 139.2, 134.5, 133.6, 132.5, 131.3, 129.8, 129.6, 128.7, 128.3 (5 carbons), 127.5 (2 carbons), 127.4 (5 carbons), 115.4 (d, J=20.7 Hz), 109.7, 104.1 (d, J=3.1 Hz), 82.8, 79.7, 76.7, 75.2, 74.6, 74.1, 73.6, 71.0, 69.4, 56.3, 55.3, 46.1, 43.0, 40.0, 38.1, 34.6, 32.8, 30.9, 26.2, 24.1, 22.4, 21.2, 20.4, 13.8, 10.6; ¹⁹F NMR (377 MHz, DMSO) δ −138.8; HRMS (ESI): caled for (C₆₁H₆₁FN₂O₁₉+H⁺), 1145.39253; found 1145.39112 [M+H]^{m}.

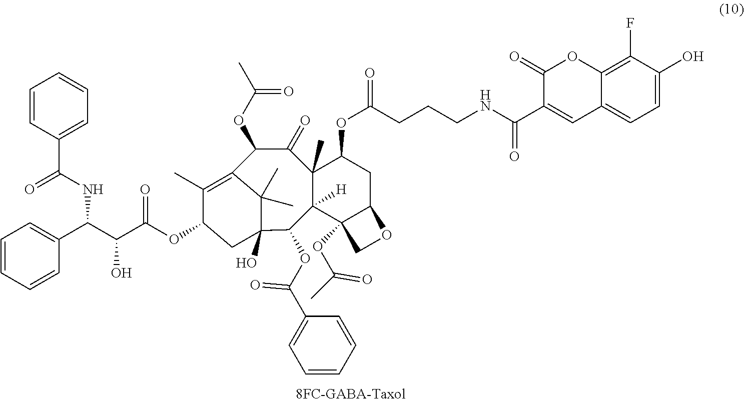

(2aR,4S,4aS,6R,9S,11S,12S,12bS)-9-(((2R,3S)-3-Benzamido-2-hydroxy-3-phenylpropanoyl)oxy)-12-(benzoyloxy)-4-((4-(8-fluoro-7-hydroxy-2-oxo-2H-chromene-3-carboxamido)butanoyl)oxy)-11-hydroxy-4a,8,13,13-tetramethyl-5-oxo-3,4,4a,5,6,9,10,11,12,12a-decahydro-1H-7,11-methanocyclodeca[3,4]benzo[1,2-b]oxete-6,12b(2aH)-diyl diacetate (8FC-GABA-Taxol, 10). To 8FC-GABA-Taxol-TBS (35, 23 mg, 18.3 μmol) in CH₂Cl₂ (2.0 mL) was added tetrabutylammonium fluoride (1 M in THF, 55 μL, 55 μmol). The reaction mixture was stirred at 22° C. for 4 h. The solvent was removed under reduced pressure and the residue was dissolved in MeOH (1 mL). The product was purified by reverse phase chromatography on a Teledyne ISCO Combiflash instrument (C18 column, H₂O and CH₃CN, 0.1% formic acid v/v, gradient: H₂O:CH₃CN (90:10) to (0:100) over 20 min, elution time 7-8 min). Pure fractions were collected and combined, and the solvent was removed using lyophilization for 16 h to afford 8FC-GABA-Taxol (10) as a colorless solid; Yield: 15 mg (72%). ¹H NMR (400 MHz, CDCl₃) δ 8.85-8.70 (m, 2H), 8.16-8.04 (m, 2H), 7.83-7.72 (m, 2H), 7.64-7.58 (m, 1H), 7.53-7.46 (m, 5H), 7.44-7.37 (m, 4H), 7.36-7.30 (m, 2H), 7.16 (d, J=8.9 Hz, 1H), 7.00 (dd, J=8.8, 7.2 Hz, 1H), 6.22 (s, 1H), 6.17 (t, J=8.9 Hz, 1H), 5.80 (dd, J=9.0, 2.7 Hz, 1H), 5.66 (d, J=6.8 Hz, 1H), 5.56 (dd, J=10.5, 7.2 Hz, 1H), 4.92 (d, J=9.2 Hz, 1H), 4.81 (d, J=2.8 Hz, 1H), 4.30 (d, J=8.5 Hz, 1H), 4.18 (d, J=8.4 Hz, 1H), 3.91 (d, J=6.8 Hz, 1H), 3.48 (q, J=6.8 Hz, 2H), 2.65-2.41 (m, 2H), 2.36 (s, 3H), 2.35-2.26 (m, 3H), 2.17 (s, 3H), 2.04-1.69 (m, 8H), 1.26 (s, 1H), 1.20 (s, 3H), 1.16 (s, 3H); ¹³C NMR (101 MHz, CDCl₃) δ 202.0, 172.5, 172.4, 170.5 (2 carbons), 169.3, 167.4, 167.0, 162.2, 160.6, 143.8, 140.5, 138.3 (d, J=245.5 Hz), 138.2, 133.9, 133.8, 133.1, 132.1, 130.3 (2 carbons), 129.2 (2 carbons), 129.1, 128.9 (4 carbons), 128.5, 127.2 (4 carbons), 125.6, 115.2, 115.1, 112.9, 84.1, 81.2, 78.7, 77.4, 76.6, 75.5, 74.5, 73.5, 72.2, 71.6, 56.3, 55.2, 47.2, 43.4, 39.3, 35.7, 33.6, 31.4, 26.7, 24.2, 22.7, 20.9 (2 carbons), 14.8, 11.0; ¹⁹F NMR (377 MHz, CDCl₃) δ −159.7; HRMS (ESI): calcd for (C₆₁H₆₁FN₂O₁₉−H⁺), 1143.37798; found 1143.37863 [M−H]⁺.

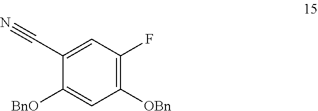 [SMILES: N#Cc1cc(F)c(OCc2ccccc2)cc1OCc1ccccc1]

2,4-Bis(benzyloxy)-5-fluorobenzonitrile (15). To a solution of 2,4,5-trifluorobenzonitrile (12, 200 mg, 1.27 mmol), in DMF (0.2 mL) was added benzyl alcohol (688 mg, 6.37 mmol) followed by potassium carbonate (757 mg, 5.47 mmol). The vessel was heated to 105° C. for 60 h, with the reaction progress monitored by TLC. After completion of the reaction, cold water was added, and the mixture was extracted with ethyl acetate (2×20 mL). The organic fractions were washed with more cold water followed by brine, dried over anhydrous Na₂SO₄, filtered, and evaporated to afford the crude product as light yellow solid. The product was purified by column chromatography on silica gel (eluent: ethyl acetate/hexane (1:19 to 1:9)) to obtain pure 15 as a pale yellow solid; Yield: 303 mg (72%); ¹H NMR (400 MHz, CDCl₃) δ 7.43-7.30 (m, 10H), 7.25 (d, J=10.2 Hz, 1H), 6.57 (d, J=6.8 Hz, 1H), 5.10 (s, 2H), 5.10 (s, 2H); ¹³C NMR (101 MHz, CDCl₃) δ 158.1, 151.6 (d, J=11.6 Hz), 146.6 (d, J=242.7 Hz), 135.5, 135.2, 129.0 (2 carbons), 128.9 (2 carbons), 128.7, 128.5, 127.5 (2 carbons), 127.1 (2 carbons), 120.0 (d, J=22.2 Hz), 115.8, 101.5, 93.5 (d, J=7.8 Hz), 71.6, 71.6; ¹⁹F NMR (377 MHz, CDCl₃) δ −141.0; HRMS (ESI): calcd for (C₂₁H₁₆FNO₂+H⁺), 334.12378; found 334.12332 ([M+H]⁺.

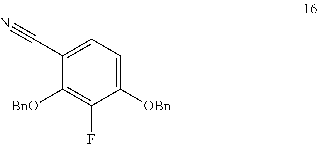 [SMILES: N#Cc1ccc(OCc2ccccc2)c(F)c1OCc1ccccc1]

2,4-Bis(benzyloxy)-3-fluorobenzonitrile (16). To a solution of 2,3,4-trifluorobenzonitrile (13, 2.5 g, 15.9 mmol), in DMF (2.5 mL) was added benzyl alcohol (4.3 g, 39.8 mmol) followed by potassium carbonate (9.47 g, 68.5 mmol). The vessel was heated to 100° C. for 16 h, with the reaction progress monitored by TLC. After completion of the reaction, cold water was added, and the mixture was extracted with ethyl acetate (2×100 mL). The organic fractions were washed with more cold water, dried over anhydrous Na₂SO₄, filtered, and evaporated to afford the crude product. The product was purified by column chromatography on silica gel (eluent: ethyl acetate/hexane (1:19)) to obtain pure 16 as a pale yellow solid; Yield: 4.4 g (83%); ¹H NMR (400 MHz, CDCl₃) δ 7.52-7.44 (m, 2H), 7.41-7.34 (m, 8H), 7.21 (dd, J=8.8, 2.1 Hz, 1H), 6.71 (dd, J=8.8, 7.2 Hz, 1H), 5.31 (s, 2H), 5.15 (s, 2H); ¹³C NMR (101 MHz, CDCl₃) δ 152.3 (d, J=9.1 Hz), 149.3 (d, J=10.2 Hz), 145.6 (d, J=248.9 Hz), 135.8, 135.3, 128.9 (2 carbons), 128.8, 128.7 (3 carbons), 128.7, 128.5 (2 carbons), 127.5 (2 carbons), 116.0 (d, J=3.6 Hz), 109.6 (d, J=1.5 Hz), 100.1 (d, J=3.3 Hz), 76.3 (d, J=6.6 Hz), 71.6; ¹⁹F NMR (377 MHz, CDCl₃) δ −148.4; HRMS (ESI): calcd for (C₂₁H₁₆FNO₂+H⁺), 334.12294; found 334.12278 ([M+H]⁺.

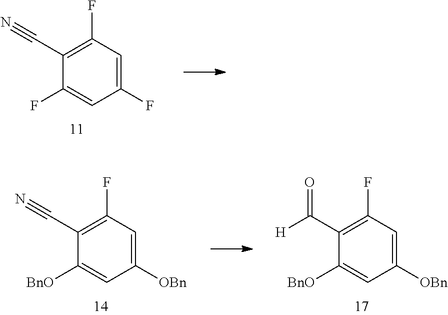 [SMILES: N#Cc1c(F)cc(F)cc1F ; N#Cc1c(F)cc(OCc2ccccc2)cc1OCc1ccccc1 ; O=Cc1c(F)cc(OCc2ccccc2)cc1OCc1ccccc1]

2,4-Bis(benzyloxy)-6-fluorobenzaldehyde (17). To a solution of 2,4,6-trifluorobenzonitrile (11, 2.5 g, 15.9 mmol) in DMF (2.5 mL) was added benzyl alcohol (4.3 g, 39.8 mmol) followed by potassium carbonate (9.47 g, 68.5 mmol). The vessel heated to 100° C. for 16 h, with reaction progress monitored by TLC. After completion of the reaction, cold water was added, and the mixture extracted with ethyl acetate (2×100 mL). The organic fractions were washed with additional cold water, dried on anhydrous Na₂SO₄, filtered, and evaporated to afford the crude product. This product was recrystallized from ethanol (10% H₂O) to provided 14 as a 2.7:1 mixture of regioisomers, favoring the desired product, that were taken forward without further characterization. To this mixture (2.96 g, 8.9 mmol) in CH₂Cl₂ (19 mL) at −78° C. was added diisobutylaluminium hydride (1.89 g, 13.3 mL, from Acros, in hexane, 1 M (for this reagent, this vendor provided the most consistent results)). The reaction mixture was stirred 3 h followed by warming to 22° C. The reaction mixture was quenched with aqueous HCl (0.5 M, 50 mL) by stirring for 1 h. The product was extracted with ethyl acetate (2×100 mL), dried over anhydrous Na₂SO₄, filtered, and evaporated under reduced pressure to afford crude 17, which was purified by silica gel column chromatography (eluent: ethyl acetate/hexane (1:9). Recrystallization from ethanol (10% H₂O) provided 17 as a colorless solid; Yield: 1.46 g, (27% over two steps); ¹H NMR (400 MHz, CDCl₃) δ 10.35 (d, J=1.4 Hz, 1H), 7.49-7.30 (m, 10H), 6.40 (dd, J=2.1, 1.2 Hz, 1H), 6.34 (dd, J=12.5, 2.2 Hz, 1H), 5.13 (s, 2H), 5.07 (s, 2H); ¹³C NMR (101 MHz, CDCl₃) δ 186.0 (d, J=2.5 Hz), 165.1 (d, J=261.7 Hz), 165.1 (d, J=15.0 Hz), 162.6 (d, J=8.4 Hz), 135.7, 135.4, 128.9 (2 carbons), 128.9 (2 carbons), 128.7, 128.5, 127.7 (2 carbons), 127.2 (2 carbons), 109.0 (d, J=9.3 Hz), 96.6 (d, J=2.9 Hz), 95.5 (d, J=25.4 Hz), 71.1, 70.8; ¹⁹F NMR (377 MHz, CDCl₃) δ −111.24; HRMS (ESI): calcd for (C₂₁H₁₇FO₃+H⁺), 337.12345; found 337.12248 [M+H]⁺.

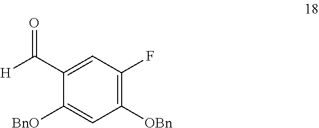 [SMILES: O=Cc1cc(F)c(OCc2ccccc2)cc1OCc1ccccc1]

2,4-Bis(benzyloxy)-5-fluorobenzaldehyde (18). To a solution of 2,4-bis(benzyloxy)-5-fluorobenzonitrile (15, 100 mg, 0.3 mmol) in CH₂Cl₂ (0.5 mL) at −78° C. was added diisobutylaluminium hydride (0.45 mL, 0.45 mmol, from Acros, in hexane, 1 M (for this reagent, this vendor provided the most consistent results)). The reaction mixture was stirred for 3 h. After warming to 22° C., the reaction mixture was quenched with aqueous HCl (0.5 M, 0.2 mL) with stirring for 1 h. The mixture was extracted with ethyl acetate (2×20 mL), and the organic layer was dried over anhydrous Na₂SO₄, filtered, and evaporated under reduced pressure to afford the crude product. Column chromatography on silica gel (eluent:ethyl acetate/Hexane (1:9) afforded 18 as a pale yellow solid; Yield: 65 mg (65%); ¹H NMR (400 MHz, CDCl₃) δ 10.34 (d, J=3.2 Hz, 1H), 7.57 (d, J=11.1 Hz, 1H), 7.46-7.30 (m, 10H), 6.61 (d, J=6.4 Hz, 1H), 5.16 (s, 2H), 5.09 (s, 2H); ¹³C NMR (176 MHz, CDCl₃) δ 187.6, 158.7, 153.0 (d, J=12.4 Hz), 147.7 (d, J=242.5 Hz), 135.8, 135.5, 129.0 (2 carbons), 128.7 (2 carbons), 128.6 (2 carbons), 127.5 (2 carbons), 127.5 (2 carbons), 118.6 (d, J=4.6 Hz), 114.8 (d, J=20.2 Hz), 100.9, 71.5 (2 carbons); ¹⁹F NMR (377 MHz, CDCl₃) δ −142.2; HRMS (ESI): calcd for (C₂₁H₁₇FO₃+H⁺), 337.12345; found 337.12293 [M+H]⁺.

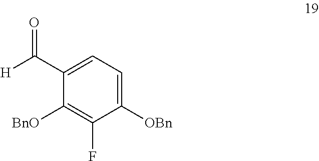 [SMILES: O=Cc1ccc(OCc2ccccc2)c(F)c1OCc1ccccc1]

2,4-Bis(benzyloxy)-3-fluorobenzaldehyde (19). To a solution of 2,4-bis(benzyloxy)-3-fluorobenzonitrile (16, 3.8 g, 11.4 mmol) in CH₂Cl₂ (10 mL) at −78° C. was added diisobutylaluminium hydride (17.1 mL, 1 M in hexanes, from Acros (for this reagent, this vendor provided the most consistent results)). The reaction mixture was stirred for 3 h. After warming to 22° C., the reaction mixture was quenched with aqueous HCl (0.5 M, 50 mL) with stirring for 1 h. The mixture was extracted with ethyl acetate (2×100 mL), and the organic layer was dried over anhydrous Na₂SO₄, filtered, and evaporated under reduced pressure to afford the crude product. Column chromatography on silica gel (eluent: ethyl acetate/Hexane (1:19) afforded a solid that was recrystallized from ethanol to obtained 19 as a colorless solid; Yield: 3.0 g (78%); ¹H NMR (400 MHz, CDCl₃) δ 10.12 (d, J=0.8 Hz, 1H), 7.55 (dd, J=8.8, 2.0 Hz, 1H), 7.49-7.30 (m, 10H), 6.86-6.77 (m, 1H), 5.30-5.26 (m, 2H), 5.22 (s, 2H); ¹³C NMR (101 MHz, CDCl₃) δ 188.2 (d, J=2.9 Hz), 153.4 (d, J=9.2 Hz), 150.0 (d, J=8.4 Hz), 145.4 (d, J=248.2 Hz), 136.0, 135.6, 128.9 (2 carbons), 128.8 (2 carbons), 128.7 (2 carbons), 128.6, 127.6 (2 carbons), 123.7, 123.7, 123.6, 109.4, 71.5 (2 carbons); ¹⁹F NMR (377 MHz, CDCl₃) δ −150.2; HRMS (ESI): calcd for (C₂₁H₁₇FO₃+H⁺), 337.12345; found 337.12250 [M+H]⁺.

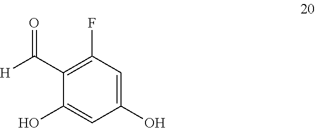 [SMILES: O=Cc1c(O)cc(O)cc1F]

2-Fluoro-4,6-dihydroxybenzaldehyde (20). To 2,4-bis(benzyloxy)-6-fluorobenzaldehyde (17, 784 mg, 2.3 mmol) in MeOH/THF (1:1, 15 mL) was added Pd(OAc)₂ (52 mg, 10 mol %). The reaction mixture was stirred under hydrogen (1 atm) for 16 h. The reaction was monitored by TLC (10% MeOH/CH₂Cl₂). After removal of the catalyst by filtration over celite, the filtrate was evaporated under reduced pressure and the product purified by column chromatography on silica gel (eluent: MeOH/CH₂Cl₂ (1% to 10%)) to obtain pure as a pale brown solid: Yield: 345 mg (95%); ¹H NMR (400 MHz, DMSO) δ 11.51 (s, 1H), 11.26 (s, 1H), 9.98 (s, 1H), 6.22-6.16 (m, 1H), 6.16-6.13 (m, 1H); ¹³C NMR (101 MHz, DMSO) δ 188.6 (d, J=6.2 Hz), 166.4 (d, J=16.4 Hz), 165.1 (d, J=255.9 Hz), 163.8 (d, J=7.4 Hz), 104.5 (d, J=11.6 Hz), 98.7 (d, J=2.7 Hz), 95.3 (d, J=22.5 Hz), ¹⁹F NMR (377 MHz, DMSO) δ −118.5; HRMS; calcd for (C₇HSFO₃−H⁺), 155.01500; found, 155.01484 [M−H]⁻.

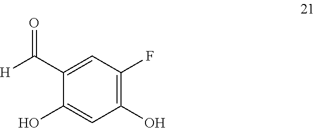 [SMILES: O=Cc1cc(F)c(O)cc1O]

5-Fluoro-2,4-dihydroxybenzaldehyde (21). To 2,4-bis(benzyloxy)-5-fluorobenzaldehyde (18, 0.4 g, 1.19 mmol) in MeOH/THF (1:1) was added Pd(OAc)₂ (40 mg, 10 mol %). The reaction mixture was stirred under hydrogen (1 atm) at 22° C. for 16 h. The reaction progress was monitored by TLC (10% ethyl acetate:hexane followed by 10% MeOH:CH₂Cl₂). After removal of the catalyst by filtration through celite, the filtrate was evaporated under reduced pressure and purified by column chromatography on silica gel (eluent: MeOH/CH₂Cl₂ (1:9)) to obtain pure 21 as a pale orange solid; Yield: 0.165 g (89%); ¹H NMR (400 MHz, DMSO) δ 11.12 (s, 1H), 10.67 (s, 1H), 9.99 (d, J=2.4 Hz, 1H), 7.36 (d, J=11.2 Hz, 1H), 6.54 (d, J=7.2 Hz, 1H); ¹³C NMR (101 MHz, DMSO) δ 189.1, 159.0, 153.0 (d, J=14.2 Hz), 145.4 (d, J=236.1 Hz), 114.8 (d, J=18.9 Hz), 113.9, 104.5; ¹⁹F NMR (377 MHz, DMSO) δ −146.27; HRMS; calcd for (C₇HFO₃−H⁺), 155.01500; found 155.01480 [M−H]⁻.

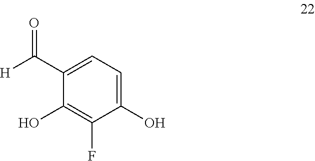 [SMILES: O=Cc1ccc(O)c(F)c1O]

3-Fluoro-2,4-dihydroxybenzaldehyde (22). To 2,4-bis(benzyloxy)-3-fluorobenzaldehyde (19, 740 mg, 2.2 mmol) in MeOH/THF (1:1, 15 mL) was added Pd(OAc)₂ (49 mg, 10 mol %). The reaction mixture was stirred under hydrogen (1 atm) for 16 h. The reaction was monitored by TLC (10% MeOH/CH₂Cl₂). After removal of the catalyst by filtration over celite, the filtrate was evaporated under reduced pressure and the product purified by using column chromatography on silica gel (eluent: MeOH/CH₂Cl₂ (1% to 10%)) to obtain pure 22 as a pale brown solid: Yield: 259 mg (75%); ¹H NMR (400 MHz, DMSO) δ 11.10 (s, 1H), 10.98 (s, 1H), 9.97 (d, J=0.9 Hz, 1H), 7.38 (dd, J=8.7, 1.8 Hz, 1H), 6.59 (dd, J=8.7, 7.3 Hz, 1H); ¹³C NMR (101 MHz, DMSO) δ 191.3 (d, J=3.0 Hz), 152.4 (d, J=9.4 Hz), 150.0 (d, J=10.9 Hz), 139.6 (d, J=238.0 Hz), 126.6 (d, J=3.6 Hz), 116.4, 109.1; ¹⁹F NMR (377 MHz, DMSO) δ −161.9; HRMS; calcd for (C₇H₅FO₃−H⁺), 155.01500; found, 155.01481 [M−H]⁻.

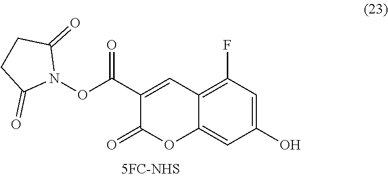 [SMILES: O=C(ON1C(=O)CCC1=O)c1cc2c(F)cc(O)cc2oc1=O]

2,5-dioxopyrrolidin-1-yl 5-fluoro-7-hydroxy-2-oxo-2H-chromene-3-carboxylate (5FC-NHS, 23). To 5-fluoro-7-hydroxy-2-oxo-2H-chromene-3-carboxylic acid (3, 75 mg, 1.23 mmol) in DMF (1.5 mL), was added N-(3-dimethylaminopropyl)-N′-ethylcarbodiimide hydrochloride, (EDC·HCl, 128 mg, 0.669 mmol) and 1-hydroxypyrrolidine-2,5-dione (77 mg, 0.669 mmol). The reaction mixture was stirred at 22° C. for 16 h and was subsequently added dropwise to cold aq. HCl (2 N, 3 mL). The yellow precipitate was filtered, washed with cold aq. HCl (2 N, 5 mL), and dried under high vacuum to afford 5FC-NHS (23) as a yellow solid. Yield: 60 mg (56%); ¹H NMR (400 MHz, DMSO) δ 11.93 (s, 1H), 8.73 (d, J=0.7 Hz, 1H), 6.76 (dd, J=11.2, 2.1 Hz, 1H), 6.69-6.59 (m, 1H), 2.88 (d, J=4.9 Hz, 4H); ¹³C NMR (101 MHz, DMSO) δ 170.2 (2 carbons), 166.6 (d, J=14.9 Hz), 160.4 (d, J=257.2 Hz), 158.4, 157.5 (d, J=7.3 Hz), 155.0, 145.0 (d, J=3.6 Hz), 106.9, 101.0 (d, J=19.3 Hz), 100.4 (d, J=21.4 Hz), 99.0 (d, J=2.9 Hz), 25.5 (2 carbons); ¹⁹F NMR (377 MHz, DMSO) δ −115.3; HRMS (ESI): calcd for (C₁₄H₈FNO₇−H⁺), 320.02120; found 320.02126 [M−H]⁻.

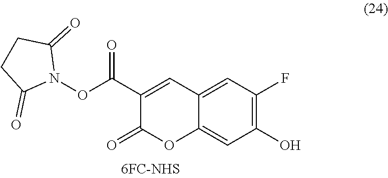 [SMILES: O=C(ON1C(=O)CCC1=O)c1cc2cc(F)c(O)cc2oc1=O]

2,5-Dioxopyrrolidin-1-yl 6-fluoro-7-hydroxy-2-oxo-2H-chromene-3-carboxylate (6FC-NHS, 24). To 6-fluoro-7-hydroxy-2-oxo-2H-chromene-3-carboxylic acid (4, 275 mg, 1.23 mmol) in DMF (3 mL), was added N-(3-dimethylaminopropyl)-N′-ethylcarbodiimide hydrochloride, (EDC·HCl, 470 mg, 2.45 mmol) and 1-hydroxypyrrolidine-2,5-dione (282 mg, 2.45 mmol). The reaction mixture was stirred at 22° C. for 16 h and was subsequently added dropwise to cold aq. HCl (2 N, 10 mL). The yellow precipitate was filtered, washed with cold aq. HCl (2 N, 10 mL), and dried under high vacuum to give 6FC-NHS (24) as a yellow solid. Yield: 350 mg, (89%); ¹H NMR (400 MHz, DMSO) δ 12.04 (s, 1H), 8.99 (s, 1H), 7.89 (d, J=10.7 Hz, 1H), 6.96 (d, J=7.1 Hz, 1H), 2.73 (d, J=0.6 Hz, 4H)¹³C NMR (101 MHz, DMSO) δ 170.3 (2 carbons), 158.6, 155.5, 154.1, 153.9 (d, J=14.6 Hz), 152.3 (d, J=3.0 Hz), 148.5 (d, J=242.0 Hz), 116.6 (d, J=20.7 Hz), 109.5 (d, J=9.1 Hz), 108.0, 104.2 (d, J=2.7 Hz), 25.5 (2 carbons); ¹⁹F NMR (377 MHz, DMSO) δ −138.9; HRMS (ESI): calcd for (C₁₄H₈FNO₇+H⁺), 322.03576; found 32.03500 [M+H]⁺.

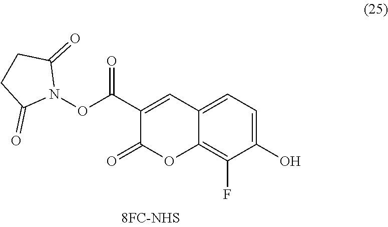 [SMILES: O=C(ON1C(=O)CCC1=O)c1cc2ccc(O)c(F)c2oc1=O]

2,5-Dioxopyrrolidin-1-yl 8-fluoro-7-hydroxy-2-oxo-2H-chromene-3-carboxylate (8FC-NHS, 25). To 8-fluoro-7-hydroxy-2-oxo-2H-chromene-3-carboxylic acid (5, 450 mg, 2.01 mmol) in DMF (3 mL), was added N-(3-dimethylaminopropyl)-N′-ethylcarbodiimide hydrochloride, (EDC·HCl, (796 mg, 4.02 mmol)) and 1-hydroxypyrrolidine-2,5-dione (462 mg, 4.02 mmol). The reaction mixture was stirred at 22° C. for 16 h and was subsequently added dropwise to cold aq. HCl (1 N, 10 mL). The yellow precipitate was filtered, washed with cold aq. HCl (1 N 5 mL), and dried under high vacuum to give 8FC-NHS (25) as a yellow solid. Yield: 430 mg (67%); ¹H NMR (400 MHz, DMSO) δ 11.90 (s, 1H), 9.06 (s, 1H), 7.76-7.66 (m, 1H), 7.10-7.00 (m, 1H), 2.89 (s, 4H); ¹³C NMR (101 MHz, DMSO) δ 170.2 (2 carbons), 158.5, 154.5, 152.9, 152.8, 145.2 (d, J=8.4 Hz), 137.3 (d, J=244.9 Hz), 127.3 (d, J=3.3 Hz), 114.8, 111.1, 107.5, 25.5 (2 carbons); ¹⁹F NMR (377 MHz, DMSO) δ −160.1; HRMS (ESI): calcd for (C₁₄H₈FNO₇+H⁺), 322.03576; found, 322.03576 [M+H]⁺.

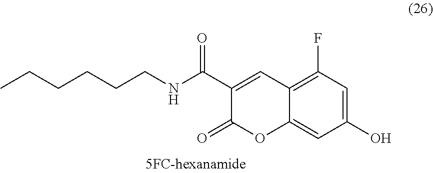 [SMILES: CCCCCCNC(=O)c1cc2c(F)cc(O)cc2oc1=O]

5-Fluoro-N-hexyl-7-hydroxy-2-oxo-2H-chromene-3-carboxamide (5FC-hexanamide, 26). To 5FC-NHS (235, 50 mg, 156 μmol) in DMF (1.5 mL) was added hexan-1-amine (19.7 mg, 195 μmol) and DIEA (30.2 mg, 233 mol). The reaction mixture was stirred at 22° C. for 16 h. The solvent was removed under reduced pressure and the residue dissolved in MeOH (1.5 mL). The product was purified by reverse phase chromatography on a Teledyne ISCO Combiflash instrument (C18 column, H₂O and CH₃CN, 0.1% formic acid v/v, gradient: H₂O:CH₃CN (90:10) to (0:100) over 20 min, elution time 10-12 min). Pure fractions were collected and combined and the solvent was removed using lyophilization for 16 h to afford 26 as a white solid; Yield: 42 mg (88%); ¹H NMR (400 MHz, DMSO) δ 8.61 (s, 1H), 8.56 (t, J=5.8 Hz, 1H), 6.73 (dd, J=11.2, 2.2 Hz, 1H), 6.67 (d, J=2.1 Hz, 1H), 3.34-3.25 (m, 2H, overlaps with water peak), 1.56-1.45 (m, 2H), 1.36-1.20 (m, 6H), 0.90-0.82 (m, 3H); ¹³C NMR (101 MHz, DMSO) δ 164.3 (d, J=14.5 Hz), 160.9, 160.4, 159.8 (d, J=254.3 Hz), 156.0 (d, J=7.7 Hz), 139.9 (d, J=3.2 Hz), 114.3, 101.3 (d, J=19.6 Hz), 100.3 (d, J=21.6 Hz), 98.7 (d, J=3.2 Hz), 39.0, 30.9, 28.9, 26.1, 22.0, 13.9; ¹⁹F NMR (377 MHz, DMSO) δ −117.2; HRMS (ESI): calcd for (C₁₆H₁₈FNO₄+H⁺), 308.12926; found 308.12854 (MH⁺).

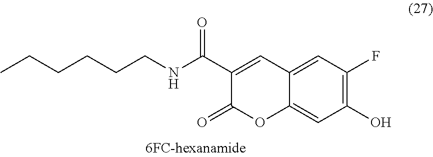 [SMILES: CCCCCCNC(=O)c1cc2cc(F)c(O)cc2oc1=O]

6-Fluoro-N-hexyl-7-hydroxy-2-oxo-2H-chromene-3-carboxamide (6FC-hexanamide, 27). To 6FC-NHS (24, 50 mg, 156 mol) in DMF (1.5 mL) was added hexan-1-amine (19.7 mg, 195 μmol) and DIEA (3.29 mg, 223 μmol). The reaction mixture was stirred at 22° C. for 16 h. The solvent was removed under reduced pressure and residue dissolved in MeOH (1.5 mL). The product was purified by reverse phase chromatography on a Teledyne ISCO Combiflash instrument (C18 column, H₂O and CH₃CN, 0.1% formic acid v/v, gradient: H₂O:CH₃CN (90:10) to (0:100) over 20 min, elution time 10-12 min). Pure fractions were collected and combined and solvent was removed by lyophilization for 16 h to afford 6FC-hexanamide (27) as a white solid; Yield: 48 mg (99%); ¹H NMR (400 MHz, DMSO) δ 11.60 (s, 1H), 8.76 (s, 1H), 8.62 (t, J=5.8 Hz, 1H), 7.84 (d, J=10.9 Hz, 1H), 6.97 (d, J=7.2 Hz, 1H), 3.30 (d, J=6.8 Hz, 2H), 1.55-1.45 (m, 2H), 1.36-1.21 (m, 6H), 0.90-0.82 (m, 3H); ¹³C NMR (101 MHz, DMSO) δ 161.2, 160.8, 152.0, 151.8 (d, J=14.6 Hz), 148.6 (d, J=241.2 Hz), 147.3 (d, J=2.8 Hz), 115.6 (d, J=20.3 Hz), 115.0, 110.2, 104.0 (d, J=2.9 Hz), 39.0, 30.9, 28.9, 26.1, 22.0, 13.9; ¹⁹F NMR (377 MHz, DMSO) δ −139.0; HRMS (ESI): calcd for (C₁₆H₁₈FNO₄+H⁺), 308.12926; found 308.12856 [M+H]⁺.

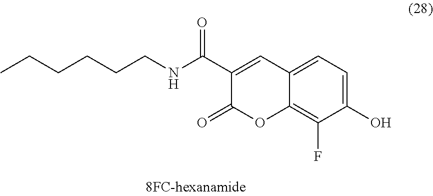 [SMILES: CCCCCCNC(=O)c1cc2ccc(O)c(F)c2oc1=O]

8-Fluoro-N-hexyl-7-hydroxy-2-oxo-2H-chromene-3-carboxamide (8FC-hexanamide, 28). To 8FC-NHS (25, 50 mg, 156 μmol) in DMF (1.5 mL) was added hexan-1-amine (19.7 mg, 195 μmol) and DIEA (30.2 mg, 223 μmol). The reaction mixture was stirred at 22° C. for 16 h. The solvent was removed under reduced pressure and the residue dissolved in MeOH (1.5 mL). The product was purified by reverse phase chromatography on a Teledyne ISCO Combiflash instrument (C18 column, H₂O and CH₃CN, 0.1% formic acid v/v, gradient: H₂O:CH₃CN (90:10) to (0:100) over 20 min, elution time 10-12 min). Pure fractions were collected and combined and the solvent was removed using lyophilization for 16 h to afford 8FC-hexanamide (28) as a white solid; Yield: 41 mg (85%); ¹H NMR (400 MHz, DMSO) δ 11.48 (s, 1H), 8.79 (d, J=1.7 Hz, 1H), 8.57 (t, J=5.8 Hz, 1H), 7.63 (dd, J=8.8, 1.7 Hz, 1H), 7.02 (dd, J=8.7, 7.6 Hz, 1H), 3.35-3.26 (m, 2H), 1.58-1.46 (m, 2H), 1.40-1.21 (m, 6H), 0.91-0.82 (m, 3H); ¹³C NMR (101 MHz, DMSO) δ 161.1, 159.9, 150.8, 150.7, 147.7 (d, J=2.6 Hz), 143.6 (d, J=8.3 Hz), 137.5 (d, J=244.9 Hz), 125.9 (d, J=4.0 Hz), 114.7 (d, J=2.6 Hz), 111.8, 39.0, 30.9, 28.9, 26.1, 22.0, 13.9; ¹⁹F NMR (377 MHz, DMSO) δ −160.0; HRMS (ESI): calcd for (C₁₆H₁₈FNO₄+H⁺), 308.12926; found 308.12849 [M+H]⁺.

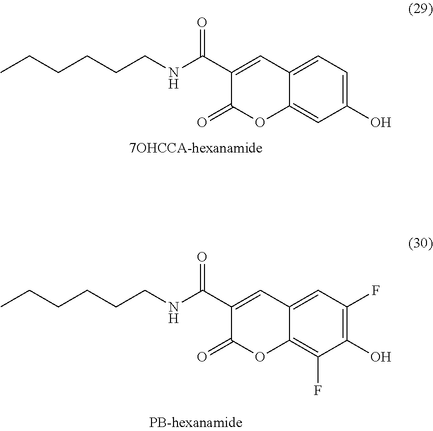 [SMILES: CCCCCCNC(=O)c1cc2ccc(O)cc2oc1=O ; CCCCCCNC(=O)c1cc2cc(F)c(O)c(F)c2oc1=O]

N-Hexyl-7-hydroxy-2-oxo-2H-chromene-3-carboxamide (29) and 6,8-Difluoro-N-hexyl-7-hydroxy-2-oxo-2H-chromene-3-carboxamide (30), These known compounds were prepared as previously described in Yin, Y et al. J. Am. Chem. Soc. 2024, 146 (1), 187-200.

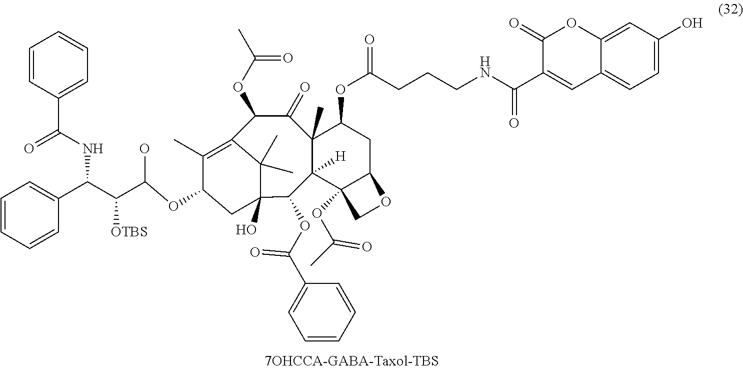

(2aR,4S,4aS,6R,9S,11S,12S,12bS)-9-(((2R,3S)-3-Benzamido-2-((tert-butyldimethylsilyl)oxy)-3-phenylpropanoyl)oxy)-12-(benzoyloxy)-11-hydroxy-4-((4-(7-hydroxy-2-oxo-2H-chromene-3-carboxamido)butanoyl)oxy)-4a,8,13,13-tetramethyl-5-oxo-3,4,4a,5,6,9,10,11,12,12a-decahydro-1H-7,11-methanocyclodeca[3,4]benzo[1,2-b]oxete-6,12b(2aH)-diyl-diacetate (7OHCCA-GABA-Taxol-TBS, 32). To a solution of H2N-GABA-Taxol-TBS (31, 40 mg, 38 mol), prepared as previously reported,^{3, 4} and 7OHCCA-NHS (TCI, 13.8 mg, 45.6 μmol) in DMF (5 mL) was added DIEA (10 μL, 57 μmol). The reaction mixture was stirred at 22° C. for 16 h. The solvent was removed under reduced pressure and the residue was dissolved in MeOH (1.5 mL). The product was purified by reverse phase chromatography on a Teledyne ISCO Combiflash instrument (C18 column, H₂O and CH₃CN, 0.1% formic acid v/v, gradient: H₂O:CH₃CN (90:10) to (0:100) over 20 min, elution time 10-12 min). Pure fractions were collected and combined, and the solvent was removed using lyophilization for 16 h to afford 7OHCCA-GABA-Taxol-TBS (32) as a white solid; Yield: 36 mg (76%); ¹H NMR (400 MHz, CDCl₃) δ 8.96 (t, J=5.9 Hz, 1H), 8.74 (s, 1H), 8.13-8.06 (m, 2H), 7.86 (s, 1H), 7.78-7.71 (m, 2H), 7.61-7.36 (m, 9H), 7.36-7.28 (m, 3H), 7.19 (d, J=8.9 Hz, 1H), 6.86-6.78 (m, 2H), 6.32-6.23 (m, 2H), 5.77-5.69 (m, 2H), 5.64 (dd, J=10.6, 7.1 Hz, 1H), 5.00-4.93 (m, 1H), 4.69 (d, J=2.2 Hz, 1H), 4.33 (d, J=8.5 Hz, 1H), 4.20 (d, J=8.5 Hz, 1H), 3.97 (d, J=6.9 Hz, 1H), 3.50 (q, J=7.3 Hz, 2H), 2.66-2.53 (m, 4H), 2.53-2.28 (m, 3H), 2.17 (s, 4H), 1.99-1.85 (m, 4H), 1.81 (s, 3H), 1.21 (s, 3H), 1.16 (s, 3H), 0.80 (s, 9H), −0.03 (s, 3H), −0.28 (s, 3H); 13C NMR (101 MHz, CDCl₃) δ 202.2, 172.5, 171.6, 170.1, 169.3, 168.0, 167.0, 163.3, 163.0, 162.7, 162.0, 156.8, 148.7, 140.9, 138.0, 133.8, 132.9, 132.3, 131.5, 130.3, 129.2, 129.0 (2 carbons), 129.0 (2 carbons), 128.8 (2 carbons), 128.3, 127.1 (2 carbons), 126.6 (2 carbons), 114.8, 113.7, 112.0, 102.9, 84.1, 81.1, 78.7, 77.4, 76.5, 75.4, 75.2, 74.7, 71.6, 71.5, 56.2, 56.1, 47.0, 43.5, 39.3, 35.7, 33.5, 31.5, 26.5, 25.7 (3 carbons), 24.3, 23.1, 21.5, 20.9, 18.2, 14.7, 11.1, −5.1, −5.7; HRMS (ESI): calcd for (C₆₇H₇₆N₂O₁₉Si−H⁺), 1239.47388; found 1239.47582 [M−H]⁻.

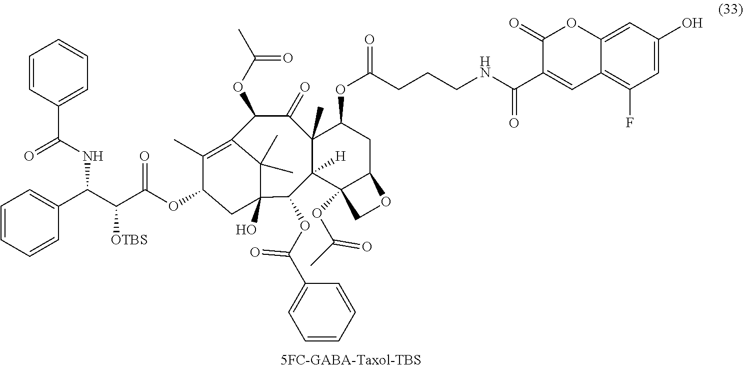

(2aR,4S,4aS,6R,9S,11S,12S,12bS)-9-(((2R,3S)-3-Benzamido-2-((tert-butyldimethylsilyl)oxy)-3-phenylpropanoyl)oxy)-12-(benzoyloxy)-4-((4-(5-fluoro-7-hydroxy-2-oxo-2H-chromene-3-carboxamido)butanoyl)oxy)-11-hydroxy-4a,8,13,13-tetramethyl-5-oxo-3,4,4a,5,6,9,10,11,12,12a-decahydro-1H-7,11-methanocyclodeca[3,4]benzo[1,2-b]oxete-6,12b(2aH)-diyl diacetate (5FC-GABA-Taxol-TBS, 33). To 5-fluoro-7-hydroxy-2-oxo-2H-chromene-3-carboxylic acid (8.5 mg, 38 μmol) in DMF (1 mL), was added N-(3-dimethylaminopropyl)-N′-ethylcarbodiimide hydrochloride, (EDC·HCl, (7.5 mg, 38 μmol) and 1-hydroxypyrrolidine-2,5-dione (5.13 mg, 38 μmol). H2N-GABA-Taxol-TBS (31, 20 mg, 19 μmol), prepared as previously reported,^{3,4} and DIEA (7 μL, 38 μmol) were added. The reaction mixture was stirred at 22° C. for 16 h. The solvent was removed under reduced pressure and the residue was dissolved in MeOH (1.5 mL). The product was purified by reverse phase chromatography on a Teledyne ISCO Combiflash instrument (C18 column, H₂O and CH₃CN, 0.1% formic acid v/v, gradient: H₂O:CH₃CN (90:10) to (0:100) over 20 min, elution time 8-9 min). Pure fractions were collected and combined, and the solvent was removed using lyophilization for 16 h to afford 5FC-GABA-Taxol-TBS (33) as a yellow solid; Yield: 16 mg (67%); ¹H NMR (400 MHz, CDCl₃) δ 8.91 (s, 1H), 8.86 (t, J=5.9 Hz, 1H), 8.14-8.04 (m, 2H), 7.89 (s, 1H), 7.78-7.71 (m, 2H), 7.62-7.28 (m, 11H), 7.16 (d, J=8.8 Hz, 1H), 6.68-6.50 (m, 2H), 6.27 (d, J=3.4 Hz, 2H), 5.76-5.58 (m, 3H), 4.95 (dd, J=9.6, 2.0 Hz, 1H), 4.69 (d, J=2.4 Hz, 1H), 4.33 (d, J=8.5 Hz, 1H), 4.20 (d, J=8.6 Hz, 1H), 3.96 (d, J=6.8 Hz, 1H), 3.50 (q, J=6.6 Hz, 2H), 2.65-2.27 (m, 6H), 2.17 (s, 4H), 2.04-1.85 (m, 4H), 1.81 (d, J=2.5 Hz, 3H), 1.21 (t, J=18.9 Hz, 8H), 0.80 (s, 9H), −0.03 (s, 3H), −0.27 (s, 3H); ¹³C NMR (101 MHz, CDCl₃) δ 202.1, 172.4, 171.4, 169.9, 169.2, 167.8, 166.9, 163.8 (d, J=14.1 Hz), 162.4, 161.2, 160.6 (d, J=257.9 Hz), 156.3, 141.9, 140.8, 137.8, 133.7, 132.8, 132.1, 130.2, 129.1, 128.9 (2 carbons), 128.9 (2 carbons), 128.8, 128.7 (2 carbons), 128.2, 127.0 (2 carbons), 126.5 (2 carbons), 113.7, 102.6 (d, J=19.6 Hz), 100.9 (d, J=22.2 Hz), 99.2, 84.0, 81.0, 78.6, 77.2, 76.4, 75.3, 75.1, 74.6, 71.5, 71.4, 56.1, 56.0, 46.9, 43.4, 39.2, 35.6, 33.4, 31.4, 26.4, 25.5 (3 carbons), 24.1, 23.0, 21.4, 20.7, 18.1, 14.6, 10.9, −5.2, −5.8; ¹⁹F NMR (377 MHz, CDCl₃) δ −114.7; HRMS (ESI): calcd for (C₆₇H₇FN₂O₁₉Si−H⁺), 1257.46446; found 1257.46430 [M−H]⁻.

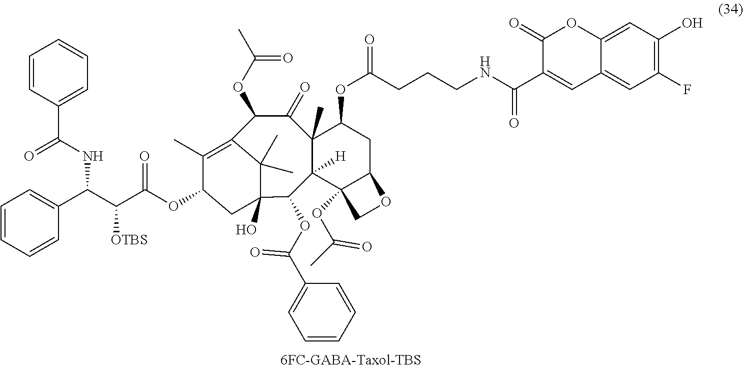

(2aR,4S,4aS,6R,9S,11S,12S,12bS)-9-(((2R,3S)-3-Benzamido-2-((tert-butyldimethylsilyl)oxy)-3-phenylpropanoyl)oxy)-12-(benzoyloxy)-4-((4-(6-fluoro-7-hydroxy-2-oxo-2H-chromene-3-carboxamido)butanoyl)oxy)-11-hydroxy-4a,8,13,13-tetramethyl-5-oxo-3,4,4a,5,6,9,10,11,12,12a-decahydro-1H-7,11-methanocyclodeca[3,4]benzo[1,2-b]oxete-6,12b(2aH)-diyl diacetate (6FC-GABA-Taxol-TBS, 34). To 6FC (4, 17 mg, 76 μmol) in DMF (2 mL), was added N-(3-dimethylaminopropyl)-N′-ethylcarbodiimide hydrochloride, (EDC·HCl, (15 mg, 76 μmol) and 1-hydroxypyrrolidine-2,5-dione (10.3 mg, 76 μmol). H₂N-GABA-Taxol-TBS (31, 40 mg, 38 μmol), prepared as previously reported^{3, 4} and DIEA (10 μL, 57 gmol) were added. The reaction mixture was stirred at 22° C. for 16 h. The solvent was removed under reduced pressure and the residue was dissolved in MeOH (1.5 mL). The product was purified by reverse phase chromatography on a Teledyne ISCO Combiflash instrument (C18 column, H₂O and CH₃CN, 0.1% formic acid v/v, gradient: H₂O:CH₃CN (90:10) to (0:100) over 20 min, elution time 8-9 min). Pure fractions were collected and combined, and the solvent was removed using lyophilization for 16 h to afford 6FC-GABA-Taxol-TBS, (34) as a yellow solid; Yield: 42 mg (88%); ¹H NMR (400 MHz, DMSO) δ 11.60 (s, 1H), 8.81 (d, J=9.4 Hz, 1H), 8.77 (s, 1H), 8.65 (t, J=6.0 Hz, 1H), 8.02-7.95 (m, 2H), 7.89-7.81 (m, 3H), 7.78-7.69 (m, 1H), 7.62 (t, J=7.7 Hz, 2H), 7.56-7.45 (m, 5H), 7.41 (t, J=7.6 Hz, 2H), 7.21 (t, J=7.4 Hz, 1H), 6.99 (d, J=7.2 Hz, 1H), 6.04 (s, 1H), 5.87 (t, J=9.1 Hz, 1H), 5.53 (t, J=8.9 Hz, 1H), 5.46-5.42 (m, 2H), 4.99 (d, J=9.4 Hz, 1H), 4.79-4.73 (m, 2H), 4.09-4.05 (m, 2H), 3.74 (d, J=7.0 Hz, 1H), 3.36-3.26 (m, 4H), 2.45 (s, 3H), 2.23 (t, J=7.4 Hz, 2H), 2.07 (s, 3H), 1.94 (dd, J=15.3, 9.2 Hz, 1H), 1.77-1.62 (m, 5H), 1.57 (s, 3H), 1.23 (s, 1H), 1.02 (s, 3H), 0.98 (s, 3H), 0.80 (s, 9H), 0.08 (s, 3H), 0.03 (s, 3H); ¹³C NMR (101 MHz, DMSO) δ 202.1, 172.0, 172.0, 170.7, 169.3, 166.7, 165.7, 161.8, 161.2, 152.5, 152.0 (d, J=14.5 Hz), 149.0 (d, J=241.6 Hz), 147.9, 140.1, 138.6, 135.0, 134.1, 133.1, 131.8, 130.3, 130.1, 129.2, 128.9, 128.7 (4 carbons), 128.6, 128.4, 127.8 (3 carbons), 116.2 (d, J=20.7 Hz), 115.7, 110.8 (d, J=8.8 Hz), 104.5 (d, J=2.6 Hz), 83.4, 80.3, 77.1, 75.9, 75.8, 75.0, 74.6, 71.5, 70.5, 57.0, 55.8, 46.4, 43.4, 38.7, 35.0, 33.4, 31.4, 26.6, 25.9, 24.5 (3 carbons), 23.3, 21.7, 20.9, 18.2 (2 carbons), 14.5, 11.1, −4.5, −4.6; ¹⁹F NMR (377 MHz, DMSO) δ −139.1; HRMS (ESI): calcd for (C₆₇H₇₅FN₂O₁₉Si+H⁺), 1259.47901; found 1259.47841 [M+H]⁺.

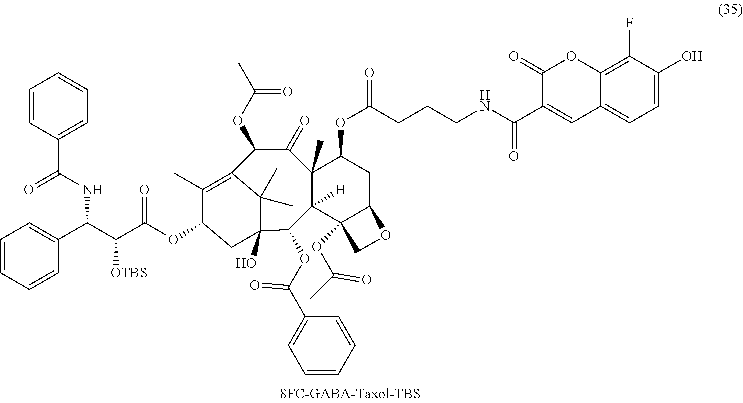

(2aR,4S,4aS,6R,9S,11S,12S,12bS)-9-(((2R,3S)-3-benzamido-2-((tert-butyldimethylsilyl)oxy)-3-phenylpropanoyl)oxy)-12-(benzoyloxy)-4-((4-(8-fluoro-7-hydroxy-2-oxo-2H-chromene-3-carboxamido)butanoyl)oxy)-11-hydroxy-4a,8,13,13-tetramethyl-5-oxo-3,4,4a,5,6,9,10,11,12,12a-decahydro-1H-7,11-methanocyclodeca[3,4]benzo[1,2-b]oxete-6,12b(2aH)-diyl diacetate (8FC-GABA-Taxol-TBS, 35). To 8FC (5, 12.8 mg, 57 μmol) in DMF (1 mL) was added N-(3-dimethylaminopropyl)-N′-ethylcarbodiimide hydrochloride, (EDC·HCl, (15 mg, 76 μmol) and 1-hydroxypyrrolidine-2,5-dione (15 mg, 76 μmol). H2N-GABA-Taxol-TBS (31, 40 mg, 38 μmol), prepared as previously reported^{3, 4} and DIEA (20 μL, 76 μmol) were added. The reaction mixture was stirred at 22° C. for 16 h. The solvent was removed under reduced pressure and the residue dissolved in MeOH (1.5 mL). The product was purified by reverse phase chromatography on a Teledyne ISCO Combiflash instrument (C18 column, H₂O and CH₃CN, 0.1% formic acid v/v, gradient: H₂O:CH₃CN (90:10) to (0:100) over 20 min, elution time 8-9 min). Pure fractions were collected and combined, and the solvent was removed using lyophilization for 16 h to afford 8FC-GABA-Taxol-TBS (35) as a yellow solid; Yield: 35 mg (73%); ¹H NMR (400 MHz, CDCl₃) δ 8.85-8.70 (m, 2H), 8.15-8.07 (m, 2H), 7.79-7.71 (m, 2H), 7.63-7.28 (m, 13H), 7.14 (d, J=8.9 Hz, 1H), 7.00-6.91 (m, 1H), 6.32-6.23 (m, 2H), 5.78-5.68 (m, 2H), 5.64 (dd, J=10.6, 7.1 Hz, 1H), 4.98 (d, J=9.2 Hz, 1H), 4.72-4.66 (m, 1H), 4.34 (d, J=8.5 Hz, 1H), 4.21 (d, J=8.5 Hz, 1H), 3.97 (d, J=6.9 Hz, 1H), 3.58-3.42 (m, 2H), 2.67-2.56 (m, 4H), 2.52-2.25 (m, 2H), 2.23-2.13 (m, 4H), 2.06-1.72 (m, 8H), 1.32-1.05 (m, 7H), 0.80 (s, 9H), −0.02 (s, 3H), −0.29 (s, 3H); ¹³C NMR (101 MHz, CDCl₃) δ 202.1, 172.4, 171.6, 170.2, 169.3, 167.7, 167.0, 162.1, 160.5, 149.8 (d, J=10.2 Hz), 148.5, 143.9 (d, J=8.7 Hz), 141.0, 138.2 (d, J=245.6 Hz), 138.2, 134.0, 133.8, 132.9, 132.1, 130.3 (2 carbons), 129.2, 129.0 (2 carbons), 128.9 (2 carbons), 128.9 (2 carbons), 128.2, 127.2 (2 carbons), 126.5 (2 carbons), 125.5 (d, J=3.9 Hz), 115.1 (d, J=13.4 Hz), 113.0, 84.2, 81.2, 78.8, 77.4, 76.6, 75.4, 75.2, 74.7, 71.5, 71.5, 56.2, 56.0, 47.1, 43.5, 39.3, 35.7, 33.5, 31.4, 26.5, 25.7 (3 carbons), 24.3, 23.1, 21.5, 20.9, 18.3, 14.8, 11.1, −5.0, −5.7; ¹⁹F NMR (377 MHz, CDCl₃) 5-159.1; HRMS (ESI): calcd for (C₆₇H₇₅FN₂O₁₉Si−H⁺), 1257.46446; found 1257.46174 [M−H]⁻.

### Synthesis and Evaluation of 6FC-Dasatinib

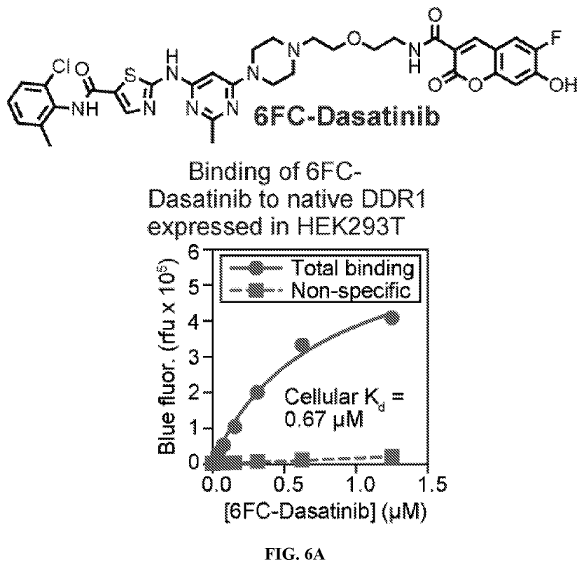

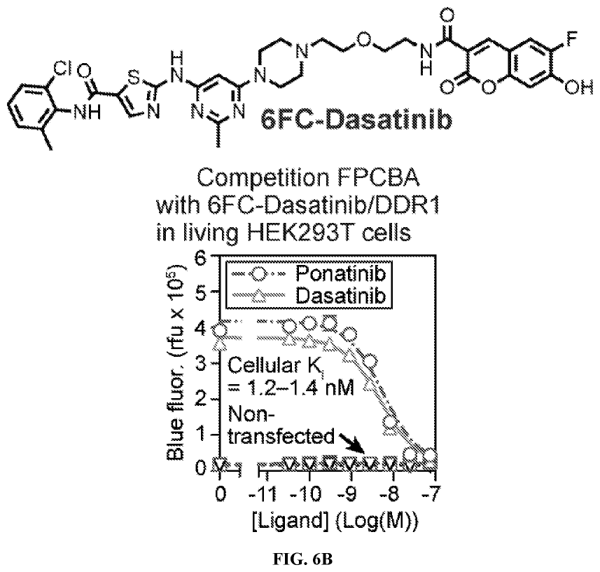

The previously reported FPCBA approach (Yin, Y. et al. J Am. Chem. Soc. 2024, 146 (1), 187-200) was used to analyze binding of small molecules to native full length DDR1 kinase overexpressed in living HEK293T cells (IRES-mVenus vector) by flow cytometry. Binding of 6FC-Dasatinib to this kinase was quantified as shown in FIG. 6A. This probe was further used to analyze competitive binding of ponatinib and dasatinib to this kinase as shown in FIG. 6B.

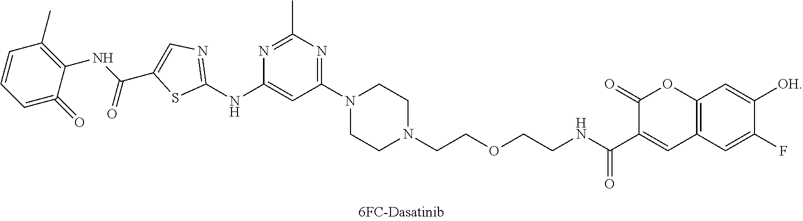

N-(2-chloro-6-methylphenyl)-2-((6-(4-(2-(2-(6-fluoro-7-hydroxy-2-oxo-2H-chromene-3-carboxamido)ethoxy)ethyl)piperazin-1-yl)-2-methylpyrimidin-4-yl)amino)thiazole-5-carboxamide (6FC-Dasatinib). To the previously reported (Cell Chemical Biology 2018, 25, 206-214) dasatinib derivative 2-((6-(4-(2-(2-aminoethoxy)ethyl)piperazin-1-yl)-2-methylpyrimidin-4-yl)amino)-N-(2-chloro-6-methylphenyl)thiazole-5-carboxamide (49 mg, 0.093 mol, 1 equiv.) in DMF (2.5 mL) was added 6FC-NHS (24, 29.8 mg, 0.093 mmol, 1 equiv) and DIEA (41 μL, 0.232 mmol, 2.5 equiv.). The reaction was stirred at room temperature under Ar overnight. Reverse-phase flash chromatography (gradient of CH₃CN in H₂O with 0.1% formic acid) yielded 6FC-Dasatinib as a yellow solid (44 mg, 65%) ¹H NMR (700 MHz, DMSO) δ 11.47 (s, 1H), 9.88 (s, 1H), 8.80 (t, J=5.5 Hz, 1H), 8.49 (s, 1H), 8.22 (s, 1H), 8.16 (s, 1H), 7.43-7.35 (m, 1H), 7.32-7.20 (m, 2H), 6.33 (d, J=7.5 Hz, 1H), 6.04 (s, 1H), 3.58 (t, J=5.7 Hz, 2H), 3.51 (dt, J=13.3, 5.7 Hz, 6H), 3.46 (t, J=5.5 Hz, 2H), 2.54 (t, 4H), 2.51 (s, 2H), 2.40 (s, 3H), 2.24 (s, 3H).¹³C NMR (176 MHz, DMSO) δ 165.1 (2 carbons), 163.3, 162.9 (additional signal from partial ionization of the phenol), 162.6, 162.4, 162.1, 159.9 (2 carbons), 156.9, 155.4, 152.2 (d, J=242.9 Hz), 146.6 (d, J=3.7 Hz), 140.8, 138.8, 133.5, 132.4, 129.0, 128.6 (d, J=8.4 Hz), 128.2, 127.0, 125.7, 113.2 (d, J=20.8 Hz), 104.3 (d, J=5.8 Hz), 82.6, 69.0, 68.3, 57.1 (2 carbons), 52.7, 43.6 (2 carbons), 38.7, 25.6, 18.3. HRMS (ESI+) m/z calcd for (C₃₄H₃₄ClFN₈O₆S+H⁺), 737.2067; found: 737.2059 [M+H]⁺.

### REFERENCES

(1) Lavis, L. D.; Raines, R. T. Bright building blocks for chemical biology. ACS Chem. Biol. 2014, 9 (4), 855-866. DOI: 10.1021/cb500078u.
(2) Offenbacher, H.; Wolfbeis, O. S.; Furlinger, E. Fluorescence Optical Sensors for Continuous Determination of near-Neutral Ph Values. Sens. Actuators 1986, 9 (1), 73-84. DOI: 10.1016/0250-6874(86)80008-7.
(3) Sun, W.-C.; Gee, K. R.; Haugland, R. P. Synthesis of novel fluorinated coumarins: Excellent UV-light excitable fluorescent dyes. Bioorg. Med. Chem. Lett. 1998, 8 (22), 3107-3110. DOI: 10.1016/s0960-894x(98)00578-2.
(4) Chen, H. M.; Nasseri, S. A.; Rahfeld, P.; Wardman, J. F.; Kohsiek, M.; Withers, S. G. Synthesis and evaluation of sensitive coumarin-based fluorogenic substrates for discovery of α-acetyl galactosaminidases through droplet-based screening. Org. Biomol. Chem. 2021, 19 (4), 789-793. DOI: 10.1039/d0ob02484h.
(5) Abrams, B.; Diwu, Z.; Guryev, O.; Aleshkov, S.; Hingorani, R.; Edinger, M.; Lee, R.; Link, J.; Dubrovsky, T. 3-Carboxy-6-chloro-7-hydroxycoumarin: a highly fluorescent, water-soluble violet-excitable dye for cell analysis. Anal. Biochem. 2009, 386 (2), 262-269. DOI: 10.1016/j.ab.2008.12.018.
(6) Cohen, J. D.; Thompson, S.; Ting, A. Y Structure-guided engineering of a Pacific Blue fluorophore ligase for specific protein imaging in living cells. Biochemistry 2011, 50 (38), 8221-8225. DOI: 10.1021/bi201037r.
(7) Lee, M. M.; Peterson, B. R. Quantification of Small Molecule-Protein Interactions using FRET between Tryptophan and the Pacific Blue Fluorophore. ACS Omega 2016, 1 (6), 1266-1276. DOI: 10.1021/acsomega.6b00356.
(8) Lee, M. M.; Gao, Z.; Peterson, B. R. Synthesis of a Fluorescent Analogue of Paclitaxel That Selectively Binds Microtubules and Sensitively Detects Efflux by P-Glycoprotein. Angew. Chem. Jnt. Ed. Engl. 2017, 56 (24), 6927-6931. DOI: 10.1002/anie.201703298.
(9) Andres, A. E.; Mariano, A.; Rane, D.; Peterson, B. R. Quantification of Engagement of Microtubules by Small Molecules in Living Cells by Flow Cytometry. ACS Bio & Med Chem Au 2022, 2 (5), 529-537. DOI: 10.1021/acsbiomedchemau.2c00031.
(10) Andres, A. E.; Rane, D.; Peterson, B. R. Pacific Blue-Taxoids as Fluorescent Molecular Probes of Microtubules. Methods. Mol. Biol. 2022, 2430, 449-466. DOI: 10.1007/978-1-0716-1983-4_28.
(11) Wu, C. P.; Calcagno, A. M.; Ambudkar, S. V. Reversal of ABC drug transporter-mediated multidrug resistance in cancer cells: evaluation of current strategies. Curr. Mol. Pharmacol. 2008, 1 (2), 93-105. DOI: 10.2174/1874467210801020093.
(12) Song, D. K.; Guo, Y; Lu, Z.; Shi, Z. Synthesis and characterization of coumarin fluorescent compounds. Jingxi Huagong 2009, 26 (6), 616-619.
(13) Chen, K. Y.; Guo, Y.; Lu, Z. H.; Yang, B. Q.; Shi, Z. Novel Coumarin-based Fluorescent Probe for Selective Detection of Bisulfite Anion in Water. Chin. J. Chem. 2010, 28 (1), 55-60. DOI: 10.1002/cjoc.201090035.
(14) Kerkovius, J. K.; Menard, F. A Practical Synthesis of 6,8-Difluoro-7-hydroxycoumarin Derivatives for Fluorescence Applications. Synthesis 2016, 48 (11), 1622-1629. DOI: 10.1055/s-0035-1561603.
(15) Ym, Y.; Zhao, S. L.; Rane, D.; Lin, Z.; Wu, M.; Peterson, B. R. Quantification of Binding of Small Molecules to Native Proteins Overexpressed in Living Cells. J. Am. Chem. Soc. 2024, 146 (1), 187-200. DOI: 10.1021/jacs.3c07488.
(16) Barasoain, I.; Diaz, J. F.; Andreu, J. M. Fluorescent taxoid probes for microtubule research. Methods Cell Biol. 2010, 95, 353-372. DOI: 10.1016/50091-679X(10)95019-X.
(17) Bhattacharyya, B.; Kapoor, S.; Panda, D. Fluorescence spectroscopic methods to analyze drug-tubulin interactions. Methods Cell Biol. 2010, 95, 301-329. DOI: 10.1016/s0091-679x(10)95017-6.
(18) Diaz, J. F.; Barasoain, I.; Andreu, J. M. Fast kinetics of Taxol binding to microtubules. Effects of solution variables and microtubule-associated proteins. J. Biol. Chem. 2003, 278 (10), 8407-8419. DOI: 10.1074/jbc.M211163200.
(19) Buey, R. M.; Calvo, E.; Barasoain, I.; Pineda, O.; Edler, M. C.; Matesanz, R.; Cerezo, G.; Vanderwal, C. D.; Day, B. W.; Sorensen, E. J.; et al. Cyclostreptin binds covalently to microtubule pores and lumenal taxoid binding sites. Nat. Chem. Biol. 2007, 3 (2), 117-125. DOI: 10.1038/nchembio853.
(20) Lukinavicius, G.; Reymond, L.; D'Este, E.; Masharina, A.; Gottfert, F.; Ta, H.; Guther, A.; Fournier, M.; Rizzo, S.; Waldmann, H.; et al. Fluorogenic probes for live-cell imaging of the cytoskeleton. Nat. Methods 2014, 11 (7), 731-733, Brief Communication. DOI: 10.1038/nmeth.2972.
(21) Pineda, J. J.; Miller, M. A.; Song, Y; Kuhn, H.; Mikula, H.; Tallapragada, N.; Weissleder, R.; Mitchison, T. J. Site occupancy calibration of taxane pharmacology in live cells and tissues. Proc. Natl. Acad. Sci. USA 2018, 115 (48), E11406-E11414. DOI: 10.1073/pnas.1800047115.
(22) Ross, J. L.; Santangelo, C. D.; Makrides, V.; Fygenson, D. K. Tau induces cooperative Taxol binding to microtubules. Proc. Natl. Acad. Sci. USA 2004, 101 (35), 12910-12915. DOI: 10.1073/pnas.0402928101.
(23) Caplow, M.; Shanks, J.; Ruhlen, R. How taxol modulates microtubule disassembly. J. Biol. Chem. 1994, 269 (38), 23399-23402. DOI: 10.1016/S0021-9258(17)31528-4 (acccessed 2021/10/22).
(24) Sharma, S.; Lagisetti, C.; Poliks, B.; Coates, R. M.; Kingston, D. G.; Bane, S. Dissecting paclitaxel-microtubule association: quantitative assessment of the 2′-OH group. Biochemistry 2013, 52 (13), 2328-2336. DOI: 10.1021/bi400014t From NLM.
(25) Andreu, J. M.; Barasoain, L The interaction of baccatin III with the taxol binding site of microtubules determined by a homogeneous assay with fluorescent taxoid. Biochemistry 2001, 40 (40), 11975-11984. DOI: 10.1021/bi010869+.
(26) Li, Y.; Edsall, R., Jr.; Jagtap, P. G.; Kingston, D. G.; Bane, S. Equilibrium studies of a fluorescent paclitaxel derivative binding to microtubules. Biochemistry 2000, 39 (3), 616-623. DOI: 10.1021/bi992044u.
(27) Matesanz, R.; Barasoain, I.; Yang, C.-G.; Wang, L.; Li, X.; de Inés, C.; Coderch, C.; Gago, F.; Barbero, J. J.; Andreu, J. M.; et al. Optimization of Taxane Binding to Microtubules: Binding Affinity Dissection and Incremental Construction of a High-Affinity Analog of Paclitaxel. Chem. Biol 2008, 15 (6), 573-585. DOI: 10.1016/j.chembiol.2008.05.008.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

1. A compound comprising one or more fluorescent moieties selected from:

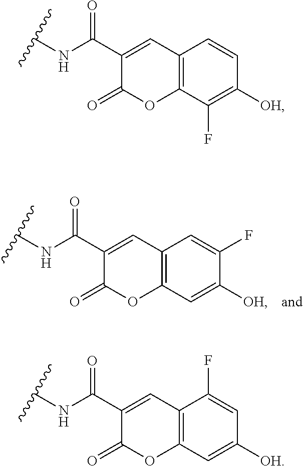 [SMILES: *NC(=O)c1cc2ccc(O)c(F)c2oc1=O ; *NC(=O)c1cc2cc(F)c(O)cc2oc1=O ; *NC(=O)c1cc2c(F)cc(O)cc2oc1=O]

2. The compound of claim 1, wherein the compound comprises a protein, a peptide, an antibody, an antigen, a receptor, a nucleic acid, a nucleotide, a nucleotide derivative, a therapeutic agent, a small molecule, a synthetic oligomer, a synthetic polymer, a hormone, a lymphokine, a cytokine, a toxin, a ligand, a carbohydrate, a sugar, an oligosaccharide, a polysaccharide, or a fatty acid.

3. The compound of claim 1, wherein the compound comprises a fluorescent probe.

4. A compound of Formula I 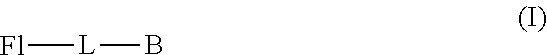
wherein:
Fl is selected from: 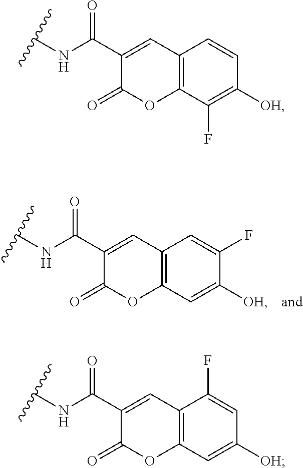 [SMILES: *NC(=O)c1cc2ccc(O)c(F)c2oc1=O ; *NC(=O)c1cc2cc(F)c(O)cc2oc1=O ; *NC(=O)c1cc2c(F)cc(O)cc2oc1=O]
L is selected from a bond or a linker moiety; and
B is a binding moiety.

5-9. (canceled)

10. The compound of claim 4, wherein L is a linker moiety selected from L1: 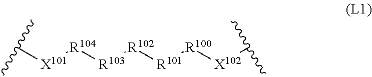
wherein:
X¹⁰¹ and X¹⁰² are independently at each occurrence selected from a bond, aryl, heteroaryl, cycloalkyl, heterocycle, NR¹³⁰C(R¹³⁰)₂, O, C(O), and S;
R¹⁰⁰, R¹⁰¹, R¹⁰², R¹⁰³, and R¹⁰⁴ are independently at each occurrence selected from the group consisting of a bond, alkyl, —C(O)—, —C(O)O—, —OC(O)—, —SO₂—, —S(O)—, C(S)—, —C(O)NR¹³⁰—, —NR¹³⁰C(O)—, —O—, —S—, —NR¹³⁰—, —C(R¹³⁰R¹³⁰)—, —P(O)(OR¹⁰⁶))—, —R(O)(OR¹⁰⁶)—, alkenyl, alkynyl, haloalkyl, alkoxy, aryl, heterocycloalkyl, cycloalkyl, heteroaryl, lactic acid, or glycolic acid, each of which may be optionally substituted with one or more (for example, 1, 2, 3, or 4) substituents independently selected from R¹⁴⁰;
R¹⁰⁶ is independently at each occurrence selected from the group consisting of hydrogen, alkyl, arylalkyl, heteroarylalkyl, alkenyl, alkynyl, aryl, heteroaryl, cycloalkyl, or heterocycloalkyl;
R¹³⁰ is independently as each occurrence selected from the group consisting of hydrogen, alkyl, cycloalkyl, heterocycloalkyl, aryl, heteroaryl, —C(O)H, —C(O)OH, —C(O)alkyl, —C(O)Oalkyl, —C(O)(cycloalkyl, heterocycloalkyl, aryl, or heteroaryl), —C(O)O(cycloalkyl, heterocycloalkyl, aryl, or heteroaryl), alkenyl, or alkynyl; and
R¹⁴⁰ is independently at each occurrence selected from the group consisting of hydrogen, alkyl, alkenyl, alkynyl, fluoro, bromo, chloro, hydroxyl, alkoxy, azide, amino, cyano, —NH(alkyl, cycloalkyl, heterocyloalkyl, aryl, or heteroaryl), —N(independently alkyl, cycloalkyl, heterocycloalkyl, aryl, or heteroaryl), —NHSO₂(alkyl, cycloalkyl, heterocycloalkyl, aryl, or heteroaryl), —N(alkyl, cycloalkyl, heterocycloalkyl, aryl, or heteroaryl)SO₂alkyl, —NHSO₂alkenyl, —N(alkyl)SO₂alkenyl, —NHSO₂alkynyl, —N(alkyl)SO₂alkynyl, haloalkyl, cycloalkyl, heterocycloalkyl, aryl, or heteroaryl.

11. The compound of claim 4, wherein L is selected from: 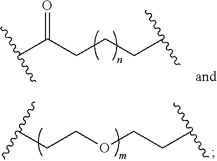
wherein:
n is an integer from 0 to 20; and
m is an integer from 0 to 5.

12. The compound of claim 4, wherein the binding moiety is capable of binding to a target molecule.

13. The compound of claim 12, wherein the target molecule comprises a target protein.

14. The compound of claim 4, wherein the binding moiety is selected from a polypeptide, a ligand, an aptamer, a nanoparticle, and a small molecule.

15. The compound of claim 4, wherein the binding moiety comprises a therapeutic agent or a derivative thereof.

16. The compound of claim 15, wherein the binding moiety comprises or is derived from an anti-cancer agent.

17. The compound of claim 15, wherein the binding moiety comprises taxol or dasatanib.

18. (canceled)

19. A compound selected from:

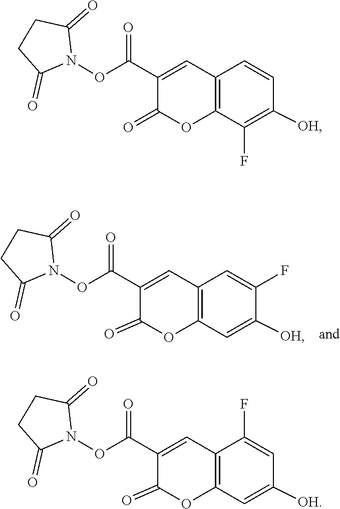 [SMILES: O=C(ON1C(=O)CCC1=O)c1cc2ccc(O)c(F)c2oc1=O ; O=C(ON1C(=O)CCC1=O)c1cc2cc(F)c(O)cc2oc1=O ; O=C(ON1C(=O)CCC1=O)c1cc2c(F)cc(O)cc2oc1=O]

20. A method of fluorescently labeling a molecule, wherein the molecule comprises one or more nucleophilic moieties, the method comprising reacting the molecule with a compound of claim 19.

21. (canceled)

22. A method of imaging a sample, the method comprising:
(a) contacting the sample with a compound of claim 1;
(b) exposing the sample to light of a wavelength that excites the one or more fluorescent moieties of the compound; and
(c) detecting an optical signal emitted by the compound.

23-26. (canceled)

27. A method of imaging a subject, the method comprising:
(a) administering to the subject a compound of claim 1, wherein upon administration the compound localizes to one or more target sites;
(b) exposing the one or more target sites to light of a wavelength that excites the one or more fluorescent moieties of the compound; and
(c) detecting an optical signal emitted by the compound.

28-30. (canceled)

31. A method of detecting the presence of a target molecule in a sample, the method comprising:
(a) contacting the sample with a compound of Formula I of claim 4;
(b) exposing the sample to light of a wavelength that excites Fl of the compound of Formula I; and
(c) detecting an optical signal emitted by the compound of Formula I, wherein the optical signal confirms the presence of the target molecule within the sample.

32-33. (canceled)

34. A method of detecting the presence of a target molecule in one or more target sites of a subject, the method comprising:
(a) administering to the subject a compound of Formula I of claim 14, wherein the compound of Formula I localizes to the one or more target sites comprising the target molecule;
(b) exposing the one or more target sites to light of a wavelength that excites Fl of the compound of Formula I; and
(c) detecting an optical signal emitted by the compound of Formula I, wherein the optical signal confirms the presence of the target molecule in the one or more target sites.

35-38. (canceled)

39. A method of determining binding affinity between a target molecule and a test compound, the method comprising:
(a) contacting the target molecule with a compound of Formula I of claim 4, wherein the compound interacts with the target molecule;
(b) measuring a first optical signal from the compound;
(c) introducing the test compound;
(d) measuring a second optical signal from the compound; and
(e) calculating a difference in the second optical signal and the first optical signal, thereby determining the binding affinity between the target molecule and the test compound.

40-44. (canceled)

45. A compound selected from:

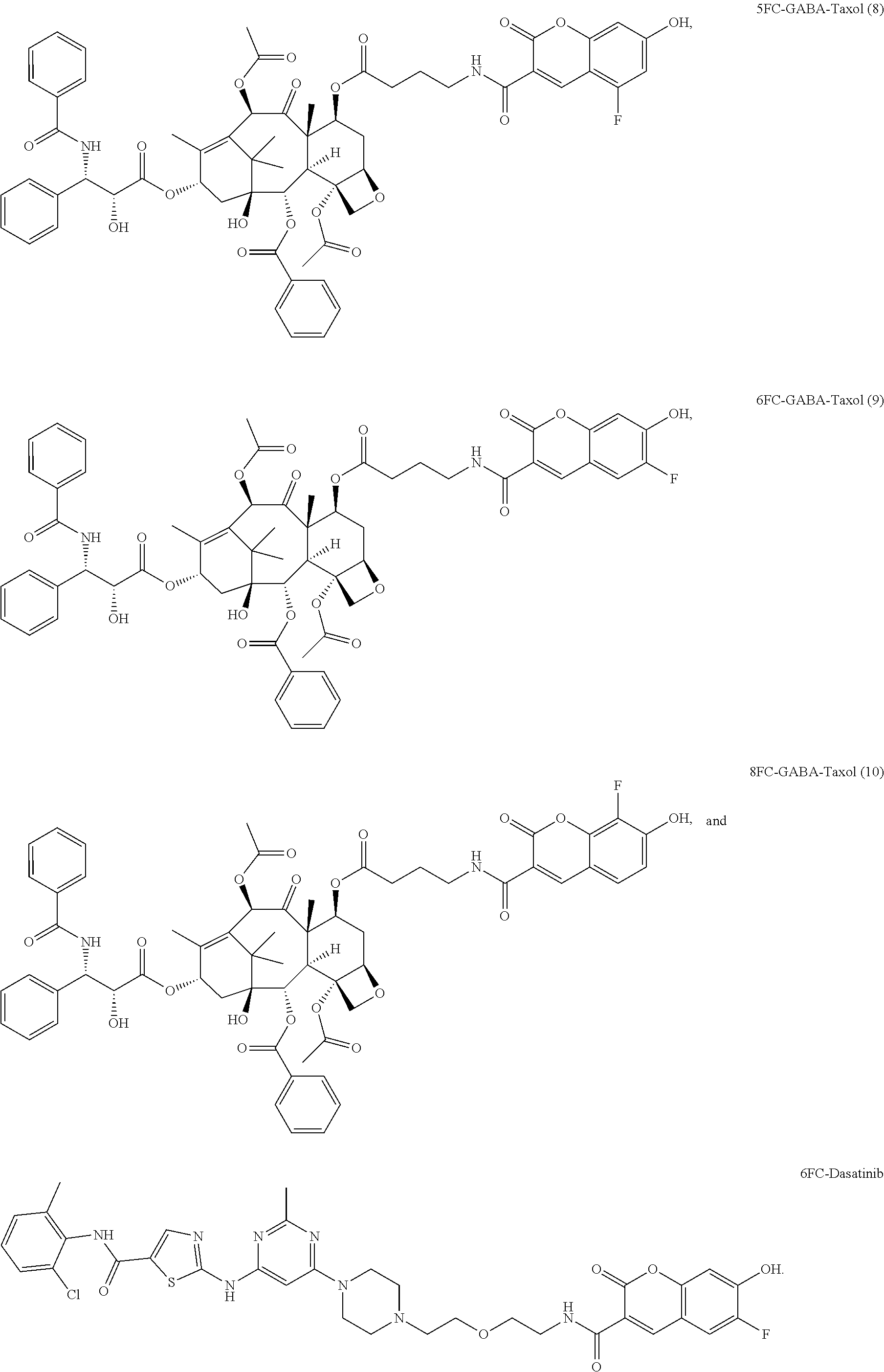

46. (canceled)
